(19) United States
(12) Patent Application Publication
(10) Pub. No.: US 2025/0313592 A1
(43) Pub. Date: Oct. 9, 2025

(54) PROCESS FOR THE PRODUCTION OF ESTETROL INTERMEDIATES

(71) Applicant: ESTETRA SRL, Liège (BE)

(72) Inventors: Amaury DUBART, Liège (BE); Damien CORNUT, Liège (BE)

(73) Assignee: ESTETRA SRL, Liège (BE)

(21) Appl. No.: 18/860,371

(22) PCT Filed: Apr. 26, 2023
(86) PCT No.: PCT/EP2023/060975

§ 371 Date: Oct. 25, 2024

(30) Foreign Application Priority Data
Apr. 26, 2022 (EP) 22170139.4
Dec. 23, 2022 (EP) 22216494.9

(51) Int. Cl. C07J 1/00; C07J 31/00

(52) U.S. Cl. CPC C07J 1/0059; C07J 1/0066; C07J 31/003

(57) ABSTRACT

The present invention relates to a process for the preparation of a compound of formula (I), a stereoisomer, a salt, a hydrate or a solvate thereof, comprising the step of desulfinylation of a compound of formula (II) to produce compound of formula (I); wherein: R¹ and R² are as defined in the claims, wherein said desulfinylation step is performed by continuous flow process.

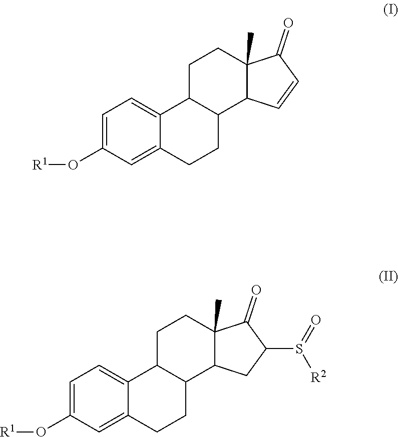

### FIELD OF THE INVENTION

The present invention relates to a new process for the synthesis of a key intermediate in the synthesis of estetrol.

### BACKGROUND OF THE INVENTION

Estrogenic substances are commonly used in methods of Hormone Replacement Therapy (HRT) and methods of female contraception. Estetrol is a biogenic estrogen that is endogenously produced by the fetal liver during human pregnancy. Recently, estetrol has been found effective as an estrogenic substance for use in HRT. Other important applications of estetrol are in the fields of contraception, therapy of auto-immune diseases, prevention and therapy of breast and colon tumors, enhancement of libido, skin care, and wound healing.

The synthesis of estetrol and derivatives thereof is known in the art. Verhaar M. T; et al (WO 2004/041839) describes a process for the preparation of estetrol starting from a 3-A-oxy-estra 1,3,5(10), 15-tetraen-17-one, wherein A is a C₁-C₅ alkyl group, or a C₇-C₁₂ benzylic group. In this document, 3-A-oxy-estra 1,3,5(10), 15-tetraen-17-ol is prepared in 6 steps from estrone where A is a benzyl group, the steps comprising protection of the 3-OH group by a benzyl group, then transformation of the 17-keto-group to a 17,17-ethylenedioxy derivative which is halogenated at the C₁₆ position using pyridinium bromide perbromide. Dehydrohalogenation is carried out by using potassium tert-butylate in dimethyl sulfoxide. Deprotection of the 17-keto-group is conducted using p-toluenesulfonic acid monohydrate in aqueous acetone. Reduction of 17-keto-group affords the 17-ol derivative.

One of the disadvantages of the process described in WO 2004/041839 is the protection of 3-OH function with a benzyl group which can be removed only by hydrogenation using Pd/C as catalyst in the last steps of the estetrol synthesis. Furthermore, the level of this catalyst in the final drug substance must be determined and must comply with the ICH guidelines. Another disadvantage of the synthesis described in WO 2004/041839 is the two steps protection/deprotection of the 17-keto function in order to generate the 15-16 double bond with a low yield.

There remains a need for an improved synthesis of 3-protected-oxy-estra-1,3,5(10), 15-tetraene-17-one. It is therefore an object of the present invention to provide a process for the preparation of 3-protected-oxy-estra-1,3,5(10), 15-tetraene-17-one which overcomes at least one of the disadvantages of the prior art.

### SUMMARY OF THE INVENTION

The present inventors have now found that this object can be obtained by using a process as defined in the appended claims. In this process, one of the reaction steps is performed by a continuous flow process. The present inventors have surprisingly found that 3-protected-oxy-estra-1,3,5(10), 15-tetraene-17-one can be obtained in good yield and with an excellent degree of purity when at least one of the synthesis steps is performed in continuous flow conditions.

According to a first aspect of the present invention, a process is provided for the preparation of a compound of formula (I), a stereoisomer, a salt, a hydrate or a solvate thereof,

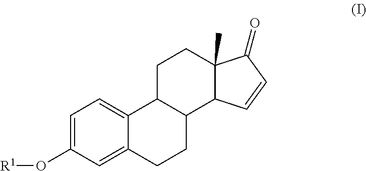

comprising the step of desulfinylation of a compound of formula (II) to produce a compound of formula (I);

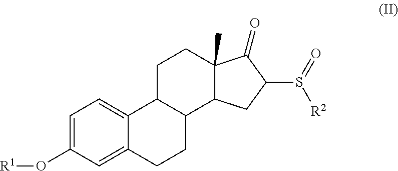

wherein:
R¹ is selected from the group comprising hydrogen, R³Si(R⁴)(R⁵)—, C₁₋₆alkyl, C₆₋₁₀aryl, C₆₋₁₀arylC₁₋₆alkyl, —CH₂—CH═CR^{a}R^{b}, C₁₋₆alkoxy, C₃₋₆cycloalkyl, and R⁶CO—; each of said C₁₋₆alkyl, C₆₋₁₀aryl, C₆₋₁₀arylC₁₋₆alkyl, and C₃₋₆cycloalkyl may be optionally substituted with one or more substituents each independently selected from the group consisting of halo, C₁₋₆alkyl, haloC₁₋₆alkyl, C₁₋₆alkoxy, haloC₁₋₆alkoxy, C₆₋₁₀aryl, heterocyclyl, and nitro;
R² is C₆₋₁₀aryl, or heteroaryl; wherein each C₆₋₁₀aryl or heteroaryl may be optionally substituted with one or more substituents each independently selected from the group consisting of halo, C₁₋₆alkyl, haloC₁₋₆alkyl, C₁₋₆alkoxy, haloC₁₋₆alkoxy, C₂₋₆alkenyl, C₂₋₆alkynyl, C₆₋₁₀aryl, heterocyclyl, heteroaryl, hydroxyl, —S(O)₂R⁷, —S(O)R⁸, —CO₂R⁹, —C(O)R¹⁰, —SR¹³, —C(O)SR¹⁴, NR¹¹R¹², cyano and nitro;
R³, R⁴ and R⁵ are each independently C₁₋₆alkyl or C₆₋₁₀aryl, wherein each C₁₋₆alkyl or C₆₋₁₀aryl may be optionally substituted with one or more substituents each independently selected from the group consisting of halo, C₁₋₆alkyl, haloC₁₋₆alkyl, C₁₋₆alkoxy, and haloC₁₋₆alkoxy;
R⁶ is C₁₋₆alkyl, C₆₋₁₀aryl, or C₃₋₆cycloalkyl, wherein each C₁₋₆alkyl, C₆₋₁₀aryl, or C₃₋₆cycloalkyl may be optionally substituted by one or more substituents independently selected from the group consisting of halo, C₁₋₆alkyl, haloC₁₋₆alkyl, C₁₋₆alkoxy, and haloC₁₋₆alkoxy;
R⁷, R⁸, R⁹ and R¹⁰ are each independently selected from the group consisting of hydrogen, hydroxyl, C₁₋₆alkyl, C₆₋₁₀aryl, and C₃₋₆cycloalkyl, wherein each C₁₋₆alkyl, C₆₋₁₀aryl, or C₃₋₆cycloalkyl may be optionally substituted by one or more substituents independently selected from the group consisting of halo, C₁₋₆alkyl, haloC₁₋₆alkyl, C₁₋₆alkoxy, and haloC₁₋₆alkoxy;
R¹¹ and R¹² are each independently selected from the group consisting of hydrogen, C₁₋₆alkyl, C₆₋₁₀aryl, C₃₋₆cycloalkyl, wherein each C₁₋₆alkyl, C₆₋₁₀aryl, or C₃₋₆cycloalkyl may be optionally substituted by one or more substituents independently selected from the group consisting of halo, C₁₋₆alkyl, haloC₁₋₆alkyl, C₁₋₆alkoxy, and haloC₁₋₆alkoxy;
R¹³ and R¹⁴ are each independently selected from the group consisting of hydrogen, C₁₋₆alkyl, C₆₋₁₀aryl, and C₃₋₆cycloalkyl, wherein each C₁₋₆alkyl, C₆₋₁₀aryl, or C₃₋₆cycloalkyl may be optionally substituted by one or more substituents independently selected from the group consisting of halo, C₁₋₆alkyl, haloC₁₋₆alkyl, C₁₋₆alkoxy, and haloC₁₋₆alkoxy;
R^{a} and R^{b} are each independently hydrogen, C₁₋₆alkyl or C₆₋₁₀aryl;
wherein said desulfinylation step is performed by a continuous flow process.

According to a second aspect, the present invention also encompasses a process for the preparation of estra-1,3,5(10),-triene 3,15α,16α,17β-tetrol of formula (E), a stereoisomer, a salt, a hydrate, an ester, an ether or a solvate thereof, said process comprising the step of preparing a compound of formula (I) by a process as described herein, and further reacting compound of formula (I) to produce estetrol of formula (E), a stereoisomer, a salt, a hydrate, an ester, an ether or a solvate thereof,

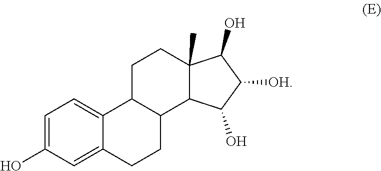 [SMILES: C[C@]12CCC3c4ccc(O)cc4CCC3C1[C@@H](O)[C@@H](O)[C@@H]2O]

According to a third aspect, the present invention also encompasses a composition obtained by the process according to the first aspect, wherein said composition comprises a compound of formula (I) and a compound of formula (IIa);

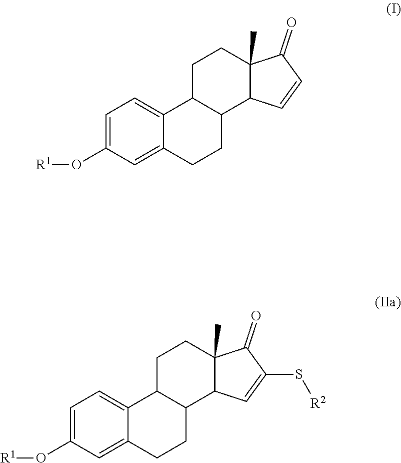

wherein R¹ and R² are as defined hereinabove.

The above and other characteristics, features and advantages of the present invention will become apparent from the following detailed description, which illustrate, by way of example, the principles of the invention.

### DETAILED DESCRIPTION OF THE INVENTION

When describing the invention, the terms used are to be construed in accordance with the following definitions, unless a context dictates otherwise.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

In the following passages, various aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Reference throughout this specification to “one embodiment” or “an embodiment” means that a particular feature, structure, or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art.

The terms “comprising”, “comprises” and “comprised of” as used herein are synonymous with “including”, “includes” or “containing”, “contains”, and are inclusive or open-ended and do not exclude additional, non-recited members, elements, or method steps. It will be appreciated that the terms “comprising”, “comprises” and “comprised of” as used herein comprise the terms “consisting of”, “consists” and “consists of”.

As used in the specification and the appended claims, the singular forms “a”, “an,” and “the” include plural referents unless the context clearly dictates otherwise. By way of example, “a step” means one step or more than one step.

Unless defined otherwise, all technical and scientific terms used herein have the same meaning as is commonly understood by one of skill in the art.

The recitation of numerical ranges by endpoints includes all integer numbers and, where appropriate, fractions subsumed within that range (e.g., 1 to 5 can include 1, 2, 3, 4 when referring to, for example, a number of elements, and can also include 1.5, 2, 2.75 and 3.80, when referring to, for example, measurements). The recitation of endpoints also includes the end point values themselves (e.g., from 1.0 to 5.0 includes both 1.0 and 5.0). Any numerical range recited herein is intended to include all sub-ranges subsumed therein.

The term “about” as used herein when referring to a measurable value such as a parameter, an amount, a temporal duration, and the like, is meant to encompass variations of +/−10% or less, preferably +/−5% or less, more preferably +/−1% or less, of and from the specified value, insofar such variations are appropriate to perform in the disclosed invention. It is to be understood that the value to which the modifier “about” refers is itself also specifically, and preferably, disclosed.

The terms “wt %”, “vol %”, or “mol %” refer to a weight percentage of a component, a volume percentage of a component, or molar percentage of a component, respectively, based on the total weight, the total volume of material, or total moles, which includes the component.

HPLC as used herein is high performance liquid chromatography utilizing UV Absorption detection with the method described in the Example section.

“Percent area of compound X” or “% area of compound X” refers to the area percentage obtained from dividing the area of the HPLC peak of compound X by the sums of areas all the HPLC peaks of compound X and each reaction product/impurity and multiplying this by 100.

“Reaction impurities” are process related impurities (by-products) including all residual starting materials, residual intermediates, and other reaction products other than compound of formula (I) detected by HPLC.

When describing the present invention, the terms used are to be construed in accordance with the following definitions, unless a context dictates otherwise.

Whenever the term “substituted” is used herein, it is meant to indicate that one or more hydrogen atoms on the atom indicated in the expression using “substituted” is replaced with a selection from the indicated group, provided that the indicated atom's normal valence is not exceeded, and that the substitution results in a chemically stable compound, i.e., a compound that is sufficiently robust to survive isolation from a reaction mixture. Where groups can be substituted, such groups may be substituted with one or more, and preferably one, two or three substituents.

The term “halo” or “halogen” as a group or part of a group is generic for fluoro, chloro, bromo, iodo.

The term “nitro” as used herein refers to the group —NO₂.

The term “amino” refers to the group —NH₂.

The term “cyano” as used herein refers to the group —CN.

The term “thiol” or “sulfhydryl” refers to the group —SH.

The term “alkyl” by itself or as part of another substituent refers to a hydrocarbyl group of formula CₙH₂ₙ₊₁ wherein n is a number greater than or equal to 1. Alkyl groups may be linear or branched and may be substituted as indicated herein. Generally, alkyl groups of this invention comprise from 1 to 6 carbon atoms, preferably from 1 to 5 carbon atoms, preferably from 1 to 4 carbon atoms, more preferably from 1 to 3 carbon atoms, still more preferably 1 to 2 carbon atoms. When a subscript is used herein following a carbon atom, the subscript refers to the number of carbon atoms that the named group may contain. For example, the term “C₁₋₆alkyl”, as a group or part of a group, refers to a hydrocarbyl group of formula CₙH₂ₙ₊₁ wherein n is a number ranging from 1 to 6. Thus, for example, “C₁₋₆alkyl” includes all linear or branched alkyl groups with between 1 and 6 carbon atoms, and thus includes methyl, ethyl, n-propyl, i-propyl, butyl and its isomers (e.g., n-butyl, i-butyl and t-butyl); pentyl and its isomers, hexyl and its isomers. For example, “C₁₋₅alkyl” includes all linear or branched alkyl groups with between 1 and 5 carbon atoms, and thus includes methyl, ethyl, n-propyl, i-propyl, butyl and its isomers (e.g., n-butyl, i-butyl and t-butyl); pentyl and its isomers. For example, “C₁₋₄alkyl” includes all linear or branched alkyl groups with between 1 and 4 carbon atoms, and thus includes methyl, ethyl, n-propyl, i-propyl, butyl and its isomers (e.g., n-butyl, i-butyl and t-butyl). For example, “C₁₋₃alkyl” includes all linear or branched alkyl groups with between 1 and 3 carbon atoms, and thus includes methyl, ethyl, n-propyl, i-propyl. A “substituted C₁₋₆alkyl” refers to a C₁₋₆alkyl group substituted with one or more substituent(s) (for example 1 to 3 substituent(s), for example 1, 2, or 3 substituent(s)) at any available point of attachment.

The term “haloC₁₋₆alkyl” as a group or part of a group, refers to a C₁₋₆alkyl group having the meaning as defined above wherein one, two, or three hydrogen atoms are each replaced with a halogen as defined herein. Non-limiting examples of such haloC₁₋₆alkyl groups include chloromethyl, 1-bromoethyl, fluoromethyl, difluoromethyl, trifluoromethyl, 1,1,1-trifluoroethyl, trichloromethyl, tribromomethyl, and the like.

The term “C₁₋₆alkoxy” or “C₁₋₆alkyloxy”, as a group or part of a group, refers to a group having the formula —OR^{b} wherein R^{b} is C₁₋₆alkyl as defined herein above. Non-limiting examples of suitable C₁₋₆alkoxy include methoxy, ethoxy, propoxy, isopropoxy, butoxy, isobutoxy, sec-butoxy, tert-butoxy, pentyloxy and hexyloxy.

The term “C₁₋₆alkylthio”, as a group or part of a group, refers to a group having the formula —S—R^{b} wherein R^{b} is C₁₋₆alkyl as defined herein above. Non-limiting examples of C₁₋₆alkylthio groups include methylthio (—SCH₃), ethylthio (—SCH₂CH₃), n-propylthio, isopropylthio, n-butylthio, isobutylthio, sec-butylthio, tert-butylthio and the like.

The term “haloC₁₋₆alkoxy”, as a group or part of a group, refers to a group of formula —O—R^{c} wherein R^{c} is haloC₁₋₆alkyl as defined herein. Non-limiting examples of suitable haloC₁₋₆alkoxy include fluoromethoxy, difluoromethoxy, trifluoromethoxy, 2,2,2-trifluoroethoxy, 1,1,2,2-tetrafluoroethoxy, 2-fluoroethoxy, 2-chloroethoxy, 2,2-difluoroethoxy, 2,2,2-trichloroethoxy, trichloromethoxy, 2-bromoethoxy, pentafluoroethyl, 3,3,3-trichloropropoxy, 4,4,4-trichlorobutoxy.

The term “cycloalkyl”, as a group or part of a group, refers to a cyclic alkyl group, that is a monovalent, saturated, hydrocarbyl group having 1 or more cyclic structure, and comprising from 3 to 12 carbon atoms, more preferably from 3 to 9 carbon atoms, more preferably from 3 to 7 carbon atoms; more preferably from 3 to 6 carbon atoms. Cycloalkyl includes all saturated hydrocarbon groups containing 1 or more rings, including monocyclic or bicyclic groups. The further rings of multi-ring cycloalkyls may be either fused, bridged and/or joined through one or more spiro atoms. When a subscript is used herein following a carbon atom, the subscript refers to the number of carbon atoms that the named group may contain. For example, the term “C₃₋₆cycloalkyl”, a cyclic alkyl group comprising from 3 to 6 carbon atoms. Examples of C₃₋₁₂cycloalkyl groups include but are not limited to cyclopropyl, cyclobutyl, cyclopentyl, cyclohexyl, cycloheptyl, cyclooctyl, bicycle[2.2.1]heptan-2yl, (1S,4R)-norbornan-2-yl, (1R,4R)-norbornan-2-yl, (1S,4S)-norbornan-2-yl, (1R,4S)-norbornan-2-yl. As used herein, the term “spiro atom” refers to the atom that connects two cyclic structures in a spiro compound. Non limiting examples of spiro atoms include quaternary carbon atoms. As used herein, the term “spiro compound” refers to a bicyclic compound wherein the two rings are connected through one atom.

The term “alkenyl” as a group or part of a group, refers to an unsaturated hydrocarbyl group, which may be linear, or branched, comprising one or more carbon-carbon double bonds. When a subscript is used herein following a carbon atom, the subscript refers to the number of carbon atoms that the named group may contain. For example, the term “C₂₋₆alkenyl” refers to an unsaturated hydrocarbyl group, which may be linear, or branched comprising one or more carbon-carbon double bonds and comprising from 2 to 6 carbon atoms. For example, C₂₋₄alkenyl includes all linear, or branched alkenyl groups having 2 to 4 carbon atoms. Examples of C₂₋₆alkenyl groups are ethenyl, 2-propenyl, 2-butenyl, 3-butenyl, 2-pentenyl and its isomers, 2-hexenyl and its isomers, 2,4-pentadienyl, and the like.

The term “alkynyl” by itself or as part of another substituent, refers to an unsaturated hydrocarbyl group, which may be linear, or branched, comprising one or more carbon-carbon triple bonds. When a subscript is used herein following a carbon atom, the subscript refers to the number of carbon atoms that the named group may contain. For example, the term “C₂₋₆alkynyl” refers to an unsaturated hydrocarbyl group, which may be linear, or branched comprising one or more carbon-carbon triple bonds and comprising from 2 to 6 carbon atoms. For example, C₂₋₄alkynyl includes all linear, or branched alkynyl groups having 2 to 4 carbon atoms. Non limiting examples of C₂₋₆alkynyl groups include ethynyl, 2-propynyl, 2-butynyl, 3-butynyl, 2-pentynyl and its chain isomers, 2-hexynyl and its chain isomers, and the like.

The term “C₆₋₁₀aryl”, as a group or part of a group, refers to a polyunsaturated, aromatic hydrocarbyl group having a single ring (i.e., phenyl) or multiple aromatic rings fused together (e.g., naphthyl), or linked covalently, typically containing 6 to 10 atoms, wherein at least one ring is aromatic. The aromatic ring may optionally include one to two additional rings (either cycloalkyl, heterocyclyl or heteroaryl) fused thereto. Examples of suitable aryl include C₆₋₁₀aryl, more preferably C₆₋₈aryl. Non-limiting examples of C₆₋₁₀aryl comprise phenyl, biphenylyl, biphenylenyl, or 1- or 2-naphthanelyl; 1-, 2-, 3-, 4-, 5- or 6-tetralinyl (also known as “1,2,3,4-tetrahydronaphtalene); 1-, 2-, 3-, 4-, 5-, 6-, 7- or 8-azulenyl, 4-, 5-, 6 or 7-indenyl; 4- or 5-indanyl; 5-, 6-, 7- or 8-tetrahydronaphthyl; 1,2,3,4-tetrahydronaphthyl; and 1,4-dihydronaphthyl; 1-, 2-, 3-, 4- or 5-pyrenyl. A “substituted C₆₋₁₀aryl” refers to a C₆₋₁₀aryl group having one or more substituent(s) (for example 1, 2 or 3 substituent(s), or 1 to 2 substituent(s)), at any available point of attachment.

The term “C₆₋₁₀arylC₁₋₆alkyl”, as a group or part of a group, means a C₁₋₆alkyl as defined herein, wherein at least one hydrogen atom is replaced by at least one C₆₋₁₀aryl as defined herein. Non-limiting examples of C₆₋₁₀arylC₁₋₆alkyl group include benzyl, phenethyl, dibenzylmethyl, methylphenylmethyl, 3-(2-naphthyl)-butyl, and the like.

The term “C₆₋₁₀arylthioC₁₋₆alkyl”, as a group or part of a group, refers to a C₁₋₆alkyl, wherein at least one hydrogen atom is replaced by at least one group having the formula —S—R^{g} wherein R^{g} is C₆₋₁₀aryl as defined herein above.

The term “heterocyclyl”, as a group or part of a group, refer to non-aromatic, fully saturated or partially unsaturated cyclic groups (for example, 3 to 7 member monocyclic, 7 to 11 member bicyclic, or comprising a total of 3 to 10 ring atoms) which have at least one heteroatom in at least one carbon atom-containing ring; wherein said ring may be fused to an aryl, cycloalkyl, heteroaryl or heterocyclyl ring. Each ring of the heterocyclyl group containing a heteroatom may have 1, 2, 3 or 4 heteroatoms selected from N, O and/or S, where the N and S heteroatoms may optionally be oxidized and the N heteroatoms may optionally be quaternized, and wherein at least one carbon atom of heterocyclyl can be oxidized to form at least one C═O. The heterocyclic group may be attached at any heteroatom or carbon atom of the ring or ring system, where valence allows. The rings of multi-ring heterocyclyl may be fused, bridged and/or joined through one or more spiro atoms.

Non limiting exemplary heterocyclyl groups include aziridinyl, oxiranyl, thiiranyl, piperidinyl, azetidinyl, oxetanyl, pyrrolidinyl, thietanyl, 2-imidazolinyl, pyrazolidinyl imidazolidinyl, isoxazolinyl, oxazolidinyl, isoxazolidinyl, thiazolidinyl, isothiazolidinyl, piperidinyl, succinimidyl, 3H-indolyl, indolinyl, chromanyl (also known as 3,4-dihydrobenzo[b]pyranyl), isoindolinyl, 2H-pyrrolyl, 1-pyrrolinyl, 2-pyrrolinyl, 3-pyrrolinyl, 4H-quinolizinyl, 2-oxopiperazinyl, piperazinyl, homopiperazinyl, 2-pyrazolinyl, 3-pyrazolinyl, tetrahydro-2H-pyranyl, 2H-pyranyl, 4H-pyranyl, 3,4-dihydro-2H-pyranyl, 3-dioxolanyl, 1,4-dioxanyl, 2,5-dioximidazolidinyl, 2-oxopiperidinyl, 2-oxopyrrolodinyl, indolinyl, tetrahydropyranyl, tetrahydrofuranyl, tetrahydrothiophenyl, tetrahydroquinolinyl, tetrahydroisoquinolin-1-yl, tetrahydroisoquinolin-2-yl, tetrahydroisoquinolin-3-yl, tetrahydroisoquinolin-4-yl, thiomorpholin-4-yl, thiomorpholin-4-ylsulfoxide, thiomorpholin-4-ylsulfone, 1,3-dioxolanyl, 1,4-oxathianyl, 1,4-dithianyl, 1,3,5-trioxanyl, 1H-pyrrolizinyl, tetrahydro-1,1-dioxothiophenyl, N-formylpiperazinyl, and morpholin-4-yl. The term “aziridinyl” as used herein includes aziridin-1-yl and aziridin-2-yl. The term “oxyranyl” as used herein includes oxyranyl-2-yl. The term “thiiranyl” as used herein includes thiiran-2-yl. The term “azetidinyl” as used herein includes azetidin-1-yl, azetidin-2-yl and azetidin-3-yl. The term “oxetanyl” as used herein includes oxetan-2-yl and oxetan-3-yl. The term “thietanyl” as used herein includes thietan-2-yl and thietan-3-yl. The term “pyrrolidinyl” as used herein includes pyrrolidin-1-yl, pyrrolidin-2-yl and pyrrolidin-3-yl. The term “tetrahydrofuranyl” as used herein includes tetrahydrofuran-2-yl and tetrahydrofuran-3-yl. The term “tetrahydrothiophenyl” as used herein includes tetrahydrothiophen-2-yl and tetrahydrothiophen-3-yl. The term “succinimidyl” as used herein includes succinimid-1-yl and succininmid-3-yl. The term “dihydropyrrolyl” as used herein includes 2,3-dihydropyrrol-1-yl, 2,3-dihydro-1H-pyrrol-2-yl, 2,3-dihydro-1H-pyrrol-3-yl, 2,5-dihydropyrrol-1-yl, 2,5-dihydro-1H-pyrrol-3-yl and 2,5-dihydropyrrol-5-yl. The term “2H-pyrrolyl” as used herein includes 2H-pyrrol-2-yl, 2H-pyrrol-3-yl, 2H-pyrrol-4-yl and 2H-pyrrol-5-yl. The term “3H-pyrrolyl” as used herein includes 3H-pyrrol-2-yl, 3H-pyrrol-3-yl, 3H-pyrrol-4-yl and 3H-pyrrol-5-yl. The term “dihydrofuranyl” as used herein includes 2,3-dihydrofuran-2-yl, 2,3-dihydrofuran-3-yl, 2,3-dihydrofuran-4-yl, 2,3-dihydrofuran-5-yl, 2,5-dihydrofuran-2-yl, 2,5-dihydrofuran-3-yl, 2,5-dihydrofuran-4-yl and 2,5-dihydrofuran-5-yl. The term “dihydrothiophenyl” as used herein includes 2,3-dihydrothiophen-2-yl, 2,3-dihydrothiophen-3-yl, 2,3-dihydrothiophen-4-yl, 2,3-dihydrothiophen-5-yl, 2,5-dihydrothiophen-2-yl, 2,5-dihydrothiophen-3-yl, 2,5-dihydrothiophen-4-yl and 2,5-dihydrothiophen-5-yl. The term “imidazolidinyl” as used herein includes imidazolidin-1-yl, imidazolidin-2-yl and imidazolidin-4-yl. The term “pyrazolidinyl” as used herein includes pyrazolidin-1-yl, pyrazolidin-3-yl and pyrazolidin-4-yl. The term “imidazolinyl” as used herein includes imidazolin-1-yl, imidazolin-2-yl, imidazolin-4-yl and imidazolin-5-yl. The term “pyrazolinyl” as used herein includes 1-pyrazolin-3-yl, 1-pyrazolin-4-yl, 2-pyrazolin-1-yl, 2-pyrazolin-3-yl, 2-pyrazolin-4-yl, 2-pyrazolin-5-yl, 3-pyrazolin-1-yl, 3-pyrazolin-2-yl, 3-pyrazolin-3-yl, 3-pyrazolin-4-yl and 3-pyrazolin-5-yl. The term “dioxolanyl” also known as “1,3-dioxolanyl” as used herein includes dioxolan-2-yl, dioxolan-4-yl and dioxolan-5-yl. The term “dioxolyl” also known as “1,3-dioxolyl” as used herein includes dioxol-2-yl, dioxol-4-yl and dioxol-5-yl. The term “oxazolidinyl” as used herein includes oxazolidin-2-yl, oxazolidin-3-yl, oxazolidin-4-yl and oxazolidin-5-yl. The term “isoxazolidinyl” as used herein includes isoxazolidin-2-yl, isoxazolidin-3-yl, isoxazolidin-4-yl and isoxazolidin-5-yl. The term “oxazolinyl” as used herein includes 2-oxazolinyl-2-yl, 2-oxazolinyl-4-yl, 2-oxazolinyl-5-yl, 3-oxazolinyl-2-yl, 3-oxazolinyl-4-yl, 3-oxazolinyl-5-yl, 4-oxazolinyl-2-yl, 4-oxazolinyl-3-yl, 4-oxazolinyl-4-yl and 4-oxazolinyl-5-yl. The term “isoxazolinyl” as used herein includes 2-isoxazolinyl-3-yl, 2-isoxazolinyl-4-yl, 2-isoxazolinyl-5-yl, 3-isoxazolinyl-3-yl, 3-isoxazolinyl-4-yl, 3-isoxazolinyl-5-yl, 4-isoxazolinyl-2-yl, 4-isoxazolinyl-3-yl, 4-isoxazolinyl-4-yl and 4-isoxazolinyl-5-yl. The term “thiazolidinyl” as used herein includes thiazolidin-2-yl, thiazolidin-3-yl, thiazolidin-4-yl and thiazolidin-5-yl. The term “isothiazolidinyl” as used herein includes isothiazolidin-2-yl, isothiazolidin-3-yl, isothiazolidin-4-yl and isothiazolidin-5-yl. The term “chromanyl” as used herein includes chroman-2-yl, chroman-3-yl, chroman-4-yl, chroman-5-yl, chroman-6-yl, chroman-7-yl and chroman-8-yl. The term “thiazolinyl” as used herein includes 2-thiazolinyl-2-yl, 2-thiazolinyl-4-yl, 2-thiazolinyl-5-yl, 3-thiazolinyl-2-yl, 3-thiazolinyl-4-yl, 3-thiazolinyl-5-yl, 4-thiazolinyl-2-yl, 4-thiazolinyl-3-yl, 4-thiazolinyl-4-yl and 4-thiazolinyl-5-yl. The term “isothiazolinyl” as used herein includes 2-isothiazolinyl-3-yl, 2-isothiazolinyl-4-yl, 2-isothiazolinyl-5-yl, 3-isothiazolinyl-3-yl, 3-isothiazolinyl-4-yl, 3-isothiazolinyl-5-yl, 4-isothiazolinyl-2-yl, 4-isothiazolinyl-3-yl, 4-isothiazolinyl-4-yl and 4-isothiazolinyl-5-yl. The term “piperidyl” also known as “piperidinyl” as used herein includes piperid-1-yl, piperid-2-yl, piperid-3-yl and piperid-4-yl. The term “dihydropyridinyl” as used herein includes 1,2-dihydropyridin-1-yl, 1,2-dihydropyridin-2-yl, 1,2-dihydropyridin-3-yl, 1,2-dihydropyridin-4-yl, 1,2-dihydropyridin-5-yl, 1,2-dihydropyridin-6-yl, 1,4-dihydropyridin-1-yl, 1,4-dihydropyridin-2-yl, 1,4-dihydropyridin-3-yl, 1,4-dihydropyridin-4-yl, 2,3-dihydropyridin-2-yl, 2,3-dihydropyridin-3-yl, 2,3-dihydropyridin-4-yl, 2,3-dihydropyridin-5-yl, 2,3-dihydropyridin-6-yl, 2,5-dihydropyridin-2-yl, 2,5-dihydropyridin-3-yl, 2,5-dihydropyridin-4-yl, 2,5-dihydropyridin-5-yl, 2,5-dihydropyridin-6-yl, 3,4-dihydropyridin-2-yl, 3,4-dihydropyridin-3-yl, 3,4-dihydropyridin-4-yl, 3,4-dihydropyridin-5-yl and 3,4-dihydropyridin-6-yl. The term “tetrahydropyridinyl” as used herein includes 1,2,3,4-tetrahydropyridin-1-yl, 1,2,3,4-tetrahydropyridin-2-yl, 1,2,3,4-tetrahydropyridin-3-yl, 1,2,3,4-tetrahydropyridin-4-yl, 1,2,3,4-tetrahydropyridin-5-yl, 1,2,3,4-tetrahydropyridin-6-yl, 1,2,3,6-tetrahydropyridin-1-yl, 1,2,3,6-tetrahydropyridin-2-yl, 1,2,3,6-tetrahydropyridin-3-yl, 1,2,3,6-tetrahydropyridin-4-yl, 1,2,3,6-tetrahydropyridin-5-yl, 1,2,3,6-tetrahydropyridin-6-yl, 2,3,4,5-tetrahydropyridin-2-yl, 2,3,4,5-tetrahydropyridin-3-yl, 2,3,4,5-tetrahydropyridin-3-yl, 2,3,4,5-tetrahydropyridin-4-yl, 2,3,4,5-tetrahydropyridin-5-yl and 2,3,4,5-tetrahydropyridin-6-yl. The term “tetrahydropyranyl” also known as “oxanyl” or “tetrahydro-2H-pyranyl”, as used herein includes tetrahydropyran-2-yl, tetrahydropyran-3-yl and tetrahydropyran-4-yl. The term “2H-pyranyl” as used herein includes 2H-pyran-2-yl, 2H-pyran-3-yl, 2H-pyran-4-yl, 2H-pyran-5-yl and 2H-pyran-6-yl. The term “4H-pyranyl” as used herein includes 4H-pyran-2-yl, 4H-pyran-3-yl and 4H-pyran-4-yl. The term “3,4-dihydro-2H-pyranyl” as used herein includes 3,4-dihydro-2H-pyran-2-yl, 3,4-dihydro-2H-pyran-3-yl, 3,4-dihydro-2H-pyran-4-yl, 3,4-dihydro-2H-pyran-5-yl and 3,4-dihydro-2H-pyran-6-yl. The term “3,6-dihydro-2H-pyranyl” as used herein includes 3,6-dihydro-2H-pyran-2-yl, 3,6-dihydro-2H-pyran-3-yl, 3,6-dihydro-2H-pyran-4-yl, 3,6-dihydro-2H-pyran-5-yl and 3,6-dihydro-2H-pyran-6-yl. The term “tetrahydrothiophenyl”, as used herein includes tetrahydrothiophen-2-yl, tetrahydrothiophenyl-3-yl and tetrahydrothiophenyl-4-yl. The term “2H-thiopyranyl” as used herein includes 2H-thiopyran-2-yl, 2H-thiopyran-3-yl, 2H-thiopyran-4-yl, 2H-thiopyran-5-yl and 2H-thiopyran-6-yl. The term “4H-thiopyranyl” as used herein includes 4H-thiopyran-2-yl, 4H-thiopyran-3-yl and 4H-thiopyran-4-yl. The term “3,4-dihydro-2H-thiopyranyl” as used herein includes 3,4-dihydro-2H-thiopyran-2-yl, 3,4-dihydro-2H-thiopyran-3-yl, 3,4-dihydro-2H-thiopyran-4-yl, 3,4-dihydro-2H-thiopyran-5-yl and 3,4-dihydro-2H-thiopyran-6-yl. The term “3,6-dihydro-2H-thiopyranyl” as used herein includes 3,6-dihydro-2H-thiopyran-2-yl, 3,6-dihydro-2H-thiopyran-3-yl, 3,6-dihydro-2H-thiopyran-4-yl, 3,6-dihydro-2H-thiopyran-5-yl and 3,6-dihydro-2H-thiopyran-6-yl. The term “piperazinyl” also known as “piperazidinyl” as used herein includes piperazin-1-yl and piperazin-2-yl. The term “morpholinyl” as used herein includes morpholin-2-yl, morpholin-3-yl and morpholin-4-yl. The term “thiomorpholinyl” as used herein includes thiomorpholin-2-yl, thiomorpholin-3-yl and thiomorpholin-4-yl. The term “dioxanyl” as used herein includes 1,2-dioxan-3-yl, 1,2-dioxan-4-yl, 1,3-dioxan-2-yl, 1,3-dioxan-4-yl, 1,3-dioxan-5-yl and 1,4-dioxan-2-yl. The term “dithianyl” as used herein includes 1,2-dithian-3-yl, 1,2-dithian-4-yl, 1,3-dithian-2-yl, 1,3-dithian-4-yl, 1,3-dithian-5-yl and 1,4-dithian-2-yl. The term “oxathianyl” as used herein includes oxathian-2-yl and oxathian-3-yl. The term “trioxanyl” as used herein includes 1,2,3-trioxan-4-yl, 1,2,3-trioxay-5-yl, 1,2,4-trioxay-3-yl, 1,2,4-trioxay-5-yl, 1,2,4-trioxay-6-yl and 1,3,4-trioxay-2-yl. The term “azepanyl” as used herein includes azepan-1-yl, azepan-2-yl, azepan-1-yl, azepan-3-yl and azepan-4-yl. The term “homopiperazinyl” as used herein includes homopiperazin-1-yl, homopiperazin-2-yl, homopiperazin-3-yl and homopiperazin-4-yl. The term “indolinyl” as used herein includes indolin-1-yl, indolin-2-yl, indolin-3-yl, indolin-4-yl, indolin-5-yl, indolin-6-yl, and indolin-7-yl. The term “quinolizinyl” as used herein includes quinolizidin-1-yl, quinolizidin-2-yl, quinolizidin-3-yl and quinolizidin-4-yl. The term “isoindolinyl” as used herein includes isoindolin-1-yl, isoindolin-2-yl, isoindolin-3-yl, isoindolin-4-yl, isoindolin-5-yl, isoindolin-6-yl, and isoindolin-7-yl. The term “3H-indolyl” as used herein includes 3H-indol-2-yl, 3H-indol-3-yl, 3H-indol-4-yl, 3H-indol-5-yl, 3H-indol-6-yl, and 3H-indol-7-yl. The term “quinolizinyl” as used herein includes quinolizidin-1-yl, quinolizidin-2-yl, quinolizidin-3-yl and quinolizidin-4-yl. The term “quinolizinyl” as used herein includes quinolizidin-1-yl, quinolizidin-2-yl, quinolizidin-3-yl and quinolizidin-4-yl. The term “tetrahydroquinolinyl” as used herein includes tetrahydroquinolin-1-yl, tetrahydroquinolin-2-yl, tetrahydroquinolin-3-yl, tetrahydroquinolin-4-yl, tetrahydroquinolin-5-yl, tetrahydroquinolin-6-yl, tetrahydroquinolin-7-yl and tetrahydroquinolin-8-yl. The term “tetrahydroisoquinolinyl” as used herein includes tetrahydroisoquinolin-1-yl, tetrahydroisoquinolin-2-yl, tetrahydroisoquinolin-3-yl, tetrahydroisoquinolin-4-yl, tetrahydroisoquinolin-5-yl, tetrahydroisoquinolin-6-yl, tetrahydroisoquinolin-7-yl and tetrahydroisoquinolin-8-yl. The term “1H-pyrrolizine” as used herein includes 1H-pyrrolizin-1-yl, 1H-pyrrolizin-2-yl, 1H-pyrrolizin-3-yl, 1H-pyrrolizin-5-yl, 1H-pyrrolizin-6-yl and 1H-pyrrolizin-7-yl. The term “3H-pyrrolizine” as used herein includes 3H-pyrrolizin-1-yl, 3H-pyrrolizin-2-yl, 3H-pyrrolizin-3-yl, 3H-pyrrolizin-5-yl, 3H-pyrrolizin-6-yl and 3H-pyrrolizin-7-yl.

The term “heteroaryl” as a group or part of a group, refers but is not limited to 5 to 12 carbon-atom aromatic rings or ring systems containing 1 or 2 rings which can be fused together or linked covalently, typically containing 5 to 6 atoms; at least one of which is aromatic in which one or more carbon atoms in one or more of these rings can be replaced by N, O and/or S atoms where the N and S heteroatoms may optionally be oxidized and the N heteroatoms may optionally be quaternized, and wherein at least one carbon atom of said heteroaryl can be oxidized to form at least one C═O. Such rings may be fused to an aryl, cycloalkyl, heteroaryl or heterocyclyl ring. Non-limiting examples of such heteroaryl, include: pyrrolyl, furanyl, thiophenyl, pyrazolyl, imidazolyl, oxazolyl, isoxazolyl, thiazolyl, isothiazolyl, triazolyl, oxadiazolyl, thiadiazolyl, tetrazolyl, oxatriazolyl, thiatriazolyl, pyridinyl, pyrimidyl, pyrazinyl, pyridazinyl, oxazinyl, dioxinyl, thiazinyl, triazinyl, benzothiazolyl, imidazo[2,1-b][1,3]thiazolyl, thieno[3,2-b]furanyl, thieno[3,2-b]thiophenyl, thieno[2,3-d][1,3]thiazolyl, thieno[2,3-d]imidazolyl, tetrazolo[1,5-a]pyridinyl, indolyl, indolizinyl, isoindolyl, benzofuranyl, isobenzofuranyl, benzothiophenyl, isobenzothiophenyl, indazolyl, benzimidazolyl, 1,3-benzoxazolyl, 1,2-benzisoxazolyl, 2,1-benzisoxazolyl, 1,3-benzothiazolyl, 1,2-benzoisothiazolyl, 2,1-benzoisothiazolyl, benzotriazolyl, 1,2,3-benzoxadiazolyl, 2,1,3-benzoxadiazolyl, 1,2,3-benzothiadiazolyl, 2,1,3-benzothiadiazolyl, benzo[d]oxazol-2(3H)-one, 2,3-dihydro-benzofuranyl, thienopyridinyl, purinyl, imidazo[1,2-a]pyridinyl, 6-oxo-pyridazin-1(6H)-yl, 2-oxopyridin-1(2H)-yl, 6-oxo-pyridazin-1(6H)-yl, 2-oxopyridin-1(2H)-yl, 1,3-benzodioxolyl, quinolinyl, isoquinolinyl, cinnolinyl, quinazolinyl, quinoxalinyl; preferably said heteroaryl group is selected from the group consisting of pyridyl, tetrazolyl, benzothiazolyl, 1,3-benzodioxolyl, benzo[d]oxazol-2(3H)-one, 2,3-dihydro-benzofuranyl, pyrazinyl, pyrazolyl, pyrrolyl, isoxazolyl, thiophenyl, imidazolyl, benzimidazolyl, pyrimidinyl, triazolyl and thiazolyl.

The term “pyrrolyl” (also called azolyl) as used herein includes pyrrol-1-yl, pyrrol-2-yl and pyrrol-3-yl. The term “furanyl” (also called “furyl”) as used herein includes furan-2-yl and furan-3-yl (also called furan-2-yl and furan-3-yl). The term “thiophenyl” (also called “thienyl”) as used herein includes thiophen-2-yl and thiophen-3-yl (also called thien-2-yl and thien-3-yl). The term “pyrazolyl” (also called 1H-pyrazolyl and 1,2-diazolyl) as used herein includes pyrazol-1-yl, pyrazol-3-yl, pyrazol-4-yl and pyrazol-5-yl. The term “imidazolyl” as used herein includes imidazol-1-yl, imidazol-2-yl, imidazol-4-yl and imidazol-5-yl. The term “oxazolyl” (also called 1,3-oxazolyl) as used herein includes oxazol-2-yl, oxazol-4-yl and oxazol-5-yl. The term “isoxazolyl” (also called 1,2-oxazolyl) as used herein includes isoxazol-3-yl, isoxazol-4-yl, and isoxazol-5-yl. The term “thiazolyl” (also called 1,3-thiazolyl) as used herein includes thiazol-2-yl, thiazol-4-yl and thiazol-5-yl (also called 2-thiazolyl, 4-thiazolyl and 5-thiazolyl). The term “isothiazolyl” (also called 1,2-thiazolyl) as used herein includes isothiazol-3-yl, isothiazol-4-yl, and isothiazol-5-yl. The term “triazolyl” as used herein includes 1H-triazolyl and 4H-1,2,4-triazolyl, “1H-triazolyl” includes 1H-1,2,3-triazol-1-yl, 1H-1,2,3-triazol-4-yl, 1H-1,2,3-triazol-5-yl, 1H-1,2,4-triazol-1-yl, 1H-1,2,4-triazol-3-yl and 1H-1,2,4-triazol-5-yl. “4H-1,2,4-triazolyl” includes 4H-1,2,4-triazol-4-yl, and 4H-1,2,4-triazol-3-yl. The term “oxadiazolyl” as used herein includes 1,2,3-oxadiazol-4-yl, 1,2,3-oxadiazol-5-yl, 1,2,4-oxadiazol-3-yl, 1,2,4-oxadiazol-5-yl, 1,2,5-oxadiazol-3-yl and 1,3,4-oxadiazol-2-yl. The term “thiadiazolyl” as used herein includes 1,2,3-thiadiazol-4-yl, 1,2,3-thiadiazol-5-yl, 1,2,4-thiadiazol-3-yl, 1,2,4-thiadiazol-5-yl, 1,2,5-thiadiazol-3-yl (also called furazan-3-yl) and 1,3,4-thiadiazol-2-yl. The term “tetrazolyl” as used herein includes 1H-tetrazol-1-yl, 1H-tetrazol-5-yl, 2H-tetrazol-2-yl, and 2H-tetrazol-5-yl. The term “oxatriazolyl” as used herein includes 1,2,3,4-oxatriazol-5-yl and 1,2,3,5-oxatriazol-4-yl. The term “thiatriazolyl” as used herein includes 1,2,3,4-thiatriazol-5-yl and 1,2,3,5-thiatriazol-4-yl. The term “pyridinyl” (also called “pyridyl”) as used herein includes pyridin-2-yl, pyridin-3-yl and pyridin-4-yl (also called 2-pyridyl, 3-pyridyl and 4-pyridyl). The term “pyrimidyl” as used herein includes pyrimid-2-yl, pyrimid-4-yl, pyrimid-5-yl and pyrimid-6-yl. The term “pyrazinyl” as used herein includes pyrazin-2-yl and pyrazin-3-yl. The term “pyridazinyl as used herein includes pyridazin-3-yl and pyridazin-4-yl. The term “oxazinyl” (also called “1,4-oxazinyl”) as used herein includes 1,4-oxazin-4-yl and 1,4-oxazin-5-yl. The term “dioxinyl” (also called “1,4-dioxinyl”) as used herein includes 1,4-dioxin-2-yl and 1,4-dioxin-3-yl. The term “thiazinyl” (also called “1,4-thiazinyl”) as used herein includes 1,4-thiazin-2-yl, 1,4-thiazin-3-yl, 1,4-thiazin-4-yl, 1,4-thiazin-5-yl and 1,4-thiazin-6-yl. The term “triazinyl” as used herein includes 1,3,5-triazin-2-yl, 1,2,4-triazin-3-yl, 1,2,4-triazin-5-yl, 1,2,4-triazin-6-yl, 1,2,3-triazin-4-yl and 1,2,3-triazin-5-yl. The term “imidazo[2,1-b][1,3]thiazolyl” as used herein includes imidazo[2,1-b][1,3]thiazoi-2-yl, imidazo[2,1-b][1,3]thiazol-3-yl, imidazo[2,1-b][1,3]thiazol-5-yl and imidazo[2,1-b][1,3]thiazol-6-yl. The term “thieno[3,2-b]furanyl” as used herein includes thieno[3,2-b]furan-2-yl, thieno[3,2-b]furan-3-yl, thieno[3,2-b]furan-4-yl, and thieno[3,2-b]furan-5-yl. The term “thieno[3,2-b]thiophenyl” as used herein includes thieno[3,2-b]thien-2-yl, thieno[3,2-b]thien-3-yl, thieno[3,2-b]thien-5-yl and thieno[3,2-b]thien-6-yl. The term “thieno[2,3-d][1,3]thiazolyl” as used herein includes thieno[2,3-d][1,3]thiazol-2-yl, thieno[2,3-d][1,3]thiazol-5-yl and thieno[2,3-d][1,3]thiazol-6-yl. The term “thieno[2,3-d]imidazolyl” as used herein includes thieno[2,3-d]imidazol-2-yl, thieno[2,3-d]imidazol-4-yl and thieno[2,3-d]imidazol-5-yl. The term “tetrazolo[1,5-a]pyridinyl” as used herein includes tetrazolo[1,5-a]pyridine-5-yl, tetrazolo[1,5-a]pyridine-6-yl, tetrazolo[1,5-a]pyridine-7-yl, and tetrazolo[1,5-a]pyridine-8-yl. The term “indolyl” as used herein includes indol-1-yl, indol-2-yl, indol-3-yl, -indol-4-yl, indol-5-yl, indol-6-yl and indol-7-yl. The term “indolizinyl” as used herein includes indolizin-1-yl, indolizin-2-yl, indolizin-3-yl, indolizin-5-yl, indolizin-6-yl, indolizin-7-yl, and indolizin-8-yl. The term “isoindolyl” as used herein includes isoindol-1-yl, isoindol-2-yl, isoindol-3-yl, isoindol-4-yl, isoindol-5-yl, isoindol-6-yl and isoindol-7-yl. The term “benzofuranyl” (also called benzo[b]furanyl) as used herein includes benzofuran-2-yl, benzofuran-3-yl, benzofuran-4-yl, benzofuran-5-yl, benzofuran-6-yl and benzofuran-7-yl. The term “isobenzofuranyl” (also called benzo[c]furanyl) as used herein includes isobenzofuran-1-yl, isobenzofuran-3-yl, isobenzofuran-4-yl, isobenzofuran-5-yl, isobenzofuran-6-yl and isobenzofuran-7-yl. The term “benzothiophenyl” (also called benzo[b]thienyl) as used herein includes 2-benzo[b]thiophenyl, 3-benzo[b]thiophenyl, 4-benzo[b]thiophenyl, 5-benzo[b]thiophenyl, 6-benzo[b]thiophenyl and -7-benzo[b]thiophenyl (also called benzothien-2-yl, benzothien-3-yl, benzothien-4-yl, benzothien-5-yl, benzothien-6-yl and benzothien-7-yl). The term “isobenzothiophenyl” (also called benzo[c]thienyl) as used herein i isobenzothien-1-yl, isobenzothien-3-yl, isobenzothien-4-yl, isobenzothien-5-yl, isobenzothien-6-yl and isobenzothien-7-yl. The term “indazolyl” (also called 1H-indazolyl or 2-azaindolyl) as used herein includes 1H-indazol-1-yl, 1H-indazol-3-yl, 1H-indazol-4-yl, 1H-indazol-5-yl, 1H-indazol-6-yl, 1H-indazol-7-yl, 2H-indazol-2-yl, 2H-indazol-3-yl, 2H-indazol-4-yl, 2H-indazol-5-yl, 2H-indazol-6-yl, and 2H-indazol-7-yl. The term “benzimidazolyl” as used herein includes benzimidazol-1-yl, benzimidazol-2-yl, benzimidazol-4-yl, benzimidazol-5-yl, benzimidazol-6-yl and benzimidazol-7-yl. The term “1,3-benzoxazolyl” as used herein includes 1,3-benzoxazol-2-yl, 1,3-benzoxazol-4-yl, 1,3-benzoxazol-5-yl, 1,3-benzoxazol-6-yl and 1,3-benzoxazol-7-yl. The term “1,2-benzisoxazolyl” as used herein includes 1,2-benzisoxazol-3-yl, 1,2-benzisoxazol-4-yl, 1,2-benzisoxazol-5-yl, 1,2-benzisoxazol-6-yl and 1,2-benzisoxazol-7-yl. The term “2,1-benzisoxazolyl” as used herein includes 2,1-benzisoxazol-3-yl, 2,1-benzisoxazol-4-yl, 2,1-benzisoxazol-5-yl, 2,1-benzisoxazol-6-yl and 2,1-benzisoxazol-7-yl. The term “1,3-benzothiazolyl” as used herein includes 1,3-benzothiazol-2-yl, 1,3-benzothiazol-4-yl, 1,3-benzothiazol-5-yl, 1,3-benzothiazol-6-yl and 1,3-benzothiazol-7-yl. The term “1,2-benzoisothiazolyl” as used herein includes 1,2-benzisothiazol-3-yl, 1,2-benzisothiazol-4-yl, 1,2-benzisothiazol-5-yl, 1,2-benzisothiazol-6-yl and 1,2-benzisothiazol-7-yl. The term “2,1-benzoisothiazolyl” as used herein includes 2,1-benzisothiazol-3-yl, 2,1-benzisothiazol-4-yl, 2,1-benzisothiazol-5-yl, 2,1-benzisothiazol-6-yl and 2,1-benzisothiazol-7-yl. The term “benzotriazolyl” as used herein includes benzotriazol-1-yl, benzotriazol-4-yl, benzotriazol-5-yl, benzotriazol-6-yl and benzotriazol-7-yl. The term “1,2,3-benzoxadiazolyl” as used herein includes 1,2,3-benzoxadiazol-4-yl, 1,2,3-benzoxadiazol-5-yl, 1,2,3-benzoxadiazol-6-yl and 1,2,3-benzoxadiazol-7-yl. The term “2,1,3-benzoxadiazolyl” as used herein includes 2,1,3-benzoxadiazol-4-yl, 2,1,3-benzoxadiazol-5-yl, 2,1,3-benzoxadiazol-6-yl and 2,1,3-benzoxadiazol-7-yl. The term “1,2,3-benzothiadiazolyl” as used herein includes 1,2,3-benzothiadiazol-4-yl, 1,2,3-benzothiadiazol-5-yl, 1,2,3-benzothiadiazol-6-yl and 1,2,3-benzothiadiazol-7-yl. The term “2,1,3-benzothiadiazolyl” as used herein includes 2,1,3-benzothiadiazol-4-yl, 2,1,3-benzothiadiazol-5-yl, 2,1,3-benzothiadiazol-6-yl and 2,1,3-benzothiadiazol-7-yl. The term “thienopyridinyl” as used herein includes thieno[2,3-b]pyridinyl, thieno[2,3-c]pyridinyl, thieno[3,2-c]pyridinyl and thieno[3,2-b]pyridinyl. The term “purinyl” as used herein includes purin-2-yl, purin-6-yl, purin-7-yl and purin-8-yl. The term “imidazo[1,2-a]pyridinyl”, as used herein includes imidazo[1,2-a]pyridin-2-yl, imidazo[1,2-a]pyridin-3-yl, imidazo[1,2-a]pyridin-4-yl, imidazo[1,2-a]pyridin-5-yl, imidazo[1,2-a]pyridin-6-yl and imidazo[1,2-a]pyridin-7-yl. The term “1,3-benzodioxolyl”, as used herein includes 1,3-benzodioxol-4-yl, 1,3-benzodioxol-5-yl, 1,3-benzodioxol-6-yl, and 1,3-benzodioxol-7-yl. The term “quinolinyl” as used herein includes quinolin-2-yl, quinolin-3-yl, quinolin-4-yl, quinolin-5-yl, quinolin-6-yl, quinolin-7-yl and quinolin-8-yl. The term “isoquinolinyl” as used herein includes isoquinolin-1-yl, isoquinolin-3-yl, isoquinolin-4-yl, isoquinolin-5-yl, isoquinolin-6-yl, isoquinolin-7-yl and isoquinolin-8-yl. The term “cinnolinyl” as used herein includes cinnolin-3-yl, cinnolin-4-yl, cinnolin-5-yl, cinnolin-6-yl, cinnolin-7-yl and cinnolin-8-yl. The term “quinazolinyl” as used herein includes quinazolin-2-yl, quinazolin-4-yl, quinazolin-5-yl, quinazolin-6-yl, quinazolin-7-yl and quinazolin-8-yl. The term “quinoxalinyl” as used herein includes quinoxalin-2-yl, quinoxalin-5-yl, and quinoxalin-6-yl.

Heteroaryl and heterocyclyl as used herein includes by way of example the groups described in Paquette, Leo A. “Principles of Modern Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; Katritzky, Alan R., Rees, C. W. and Scriven, E. “Comprehensive Heterocyclic Chemistry” (Pergamon Press, 1996); and J. Am. Chem. Soc. (1960) 82:5566.

Any substituent designation that is found in more than one site in a compound of this invention shall be independently selected.

As used herein, the term “salt” refers to a chemical compound consisting of an ionic assembly of positively charged cations and negatively charged anions. Some preferred, but non-limiting examples of suitable organic and/or inorganic acids or bases are as sodium hydroxide, potassium hydroxide, ammonium hydroxide, tetrabutylammonium hydroxide, hydrochloric acid, hydrobromic acid, sulfuric acid, nitric acid, acetic acid and citric acid.

When the compounds contain an acidic group as well as a basic group the compounds may also form internal salts, and such compounds are within the scope of the invention. When the compounds contain a hydrogen-donating heteroatom, the invention also covers salts and/or isomers formed by transfer of said hydrogen atom to a basic group or atom within the molecule.

Salts of compounds of formula (I) may be prepared by one or more of these methods:
(i) by reacting the compound of formula (I) with the desired acid;
(ii) by reacting the compound of formula (I) with the desired base;
(iii) by removing an acid- or base-labile protecting group from a suitable precursor of the compound of formula (I) or by ring-opening a suitable cyclic precursor, for example, a lactone or lactam, using the desired acid; or
(iv) by converting one salt of the compound of formula (I) to another by reaction with an appropriate acid or by means of a suitable ion exchange column.

All these reactions are typically carried out in solution. The salt may precipitate from solution and be collected by filtration or may be recovered by evaporation of the solvent. The degree of ionization in the salt may vary from completely ionized to almost non-ionized.

Salts of the compounds of formula (I) include the acid addition and base salts thereof. Suitable acid addition salts are formed from acids which form non-toxic salts. Examples include the acetate, adipate, aspartate, benzoate, besylate, bicarbonate/carbonate, bisulfate/sulfate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts. Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts. Hemisalts of acids and bases may also be formed, for example, hemisulfate and hemicalcium salts.

The term “continuous flow conditions” encompasses processes developed in continuous flow micro- and/or macro/mesofluidic reactors. These continuous flow processes may encompass one or several chemical steps or downstream processing steps in a fluidic module or a combination of fluidic modules fluidically connected in series or in parallel. A microfluidic reactor is a type of continuous flow reactor encompassing a reaction channel with an internal dimension ranging from 1 to 800 micrometers, but preferably between 100-800 micrometers and more specifically between 500 and 750 micrometers and with an internal volume ranging from 0.1 to 20 mL, but preferably between 0.5 and 15 mL and more specifically between 0.75 and 10 mL. A microfluidic reactor can be constructed from various materials, including, but not restricted to, stainless steel, copper, alloys, glass, ceramics and polymer materials. A macro/mesofluidic reactor is a type of continuous flow reactor encompassing a reaction channel with internal dimension ranging from 800 to 50000 micrometers, preferably ranging from 800 and 20000 micrometers, preferably ranging from 800 and 15000 micrometers, for example ranging from 800 to 5000 micrometers, for example ranging from between 800 and 2000 micrometers and for example ranging from 800 and 1500 micrometers; and with an internal volume ranging from 1 to 20000 mL, preferably ranging from 1 to 18000, preferably ranging from 1 to 15000 mL, preferably ranging from 1 to 10000 mL, preferably ranging from 1 to 12000, preferably ranging from 1 to 6000 mL, preferably ranging from 2.5 and 500 mL and preferably ranging from 5 and 200 mL. A macro/mesofluidic reactor can be constructed from various materials, including, but not restricted to, stainless steel, copper, alloys, glass, ceramics and polymer materials. Downstream processing steps are defined as post-reactional treatment, including, but not restricted to, extractions, separation, adding a solvent or an additive, precipitating, filtrating, drying and on-line analysing. A fluidic module can be composed either of a microfluidic or a macro/mesofluidic reactor. Fluidic modules can be fluidically connected in series or in parallel. Fluidic modules can be integrated with static mixers, heat exchangers, injection points, sampling valves, pressure regulators and in-line analytics.

Preferred statements (features) and embodiments and uses of this invention are set herein below. Each statement and embodiment of the invention so defined may be combined with any other statement and/or embodiment unless clearly indicated to the contrary. In particular, any feature indicated as being preferred or advantageous may be combined with any other feature or features or statements indicated as being preferred or advantageous.

Hereto, the present invention is in particular captured by any one or any combination of one or more of the below numbered statements and embodiments, with any other aspect and/or embodiment.

1. A process for the preparation of a compound of formula (I), a stereoisomer, a salt, a hydrate or a solvate thereof,

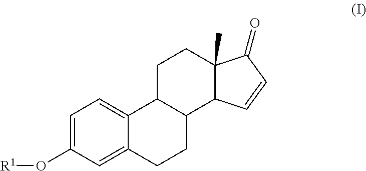

comprising the step of desulfinylation of a compound of formula (II) to produce a compound of formula (I);

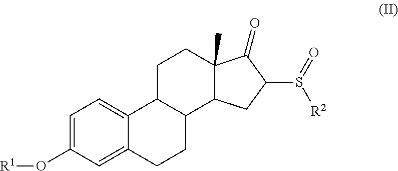

wherein:
R¹ is selected from the group comprising hydrogen, R³Si(R⁴)(R⁵)—, C₁₋₆alkyl, C₆₋₁₀aryl, C₆₋₁₀arylC₁₋₆alkyl, —CH₂—CH═CR^{a}R^{b}, C₁₋₆alkoxy, C₃₋₆cycloalkyl, and R⁶CO—; each of said C₁₋₆alkyl, C₆₋₁₀aryl, C₆₋₁₀arylC₁₋₆alkyl, and C₃₋₆cycloalkyl may be optionally substituted with one or more substituents each independently selected from the group consisting of halo, C₁₋₆alkyl, haloC₁₋₆alkyl, C₁₋₆alkoxy, haloC₁₋₆alkoxy, C₆₋₁₀aryl, heterocyclyl, and nitro;
R² is C₆₋₁₀aryl, or heteroaryl; wherein each C₆₋₁₀aryl or heteroaryl may be optionally substituted with one or more substituents each independently selected from the group consisting of halo, C₁₋₆alkyl, haloC₁₋₆alkyl, C₁₋₆alkoxy, haloC₁₋₆alkoxy, C₂₋₆alkenyl, C₂₋₆alkynyl, C₆₋₁₀aryl, heterocyclyl, heteroaryl, hydroxyl, —S(O)₂R⁷, —S(O)R⁸, CO₂R⁹, C(O)R¹⁰, —SR¹³, —C(O)SR¹⁴, NR¹¹R¹², cyano and nitro; preferably selected from the group consisting of fluoro, chloro, bromo, nitro, C₁₋₄alkyl, C₁₋₄alkyloxy, haloC₁₋₄alkyloxy;
R³, R⁴ and R⁵ are each independently C₁₋₆alkyl or C₆₋₁₀aryl, wherein each C₁₋₆alkyl or C₆₋₁₀aryl may be optionally substituted with one or more substituents each independently selected from the group consisting of halo, C₁₋₆alkyl, haloC₁₋₆alkyl, C₁₋₆alkoxy, and haloC₁₋₆alkoxy; preferably fluoro, chloro or C₁₋₄alkyl;
R⁶ is C₁₋₆alkyl, C₆₋₁₀aryl, or C₃₋₆cycloalkyl, wherein each C₁₋₆alkyl, C₆₋₁₀aryl, or C₃₋₆cycloalkyl may be optionally substituted by one or more substituents independently selected from the group consisting of halo, C₁₋₆alkyl, haloC₁₋₆alkyl, C₁₋₆alkoxy, and haloC₁₋₆alkoxy; preferably fluoro, chloro or C₁₋₄alkyl;
R⁷, R⁸, R⁹ and R¹⁰ are each independently selected from the group consisting of hydrogen, hydroxyl, C₁₋₆alkyl, C₆₋₁₀aryl, and C₃₋₆cycloalkyl, wherein each C₁₋₆alkyl, C₆₋₁₀aryl, or C₃₋₆cycloalkyl may be optionally substituted by one or more substituents independently selected from the group consisting of halo, C₁₋₆alkyl, haloC₁₋₆alkyl, C₁₋₆alkoxy, and haloC₁₋₆alkoxy;
R¹¹ and R¹² are each independently selected from the group consisting of hydrogen, C₁₋₆alkyl, C₆₋₁₀aryl, C₃₋₆cycloalkyl, wherein each C₁₋₆alkyl, C₆₋₁₀aryl, or C₃₋₆cycloalkyl may be optionally substituted by one or more substituents independently selected from the group consisting of halo, C₁₋₆alkyl, haloC₁₋₆alkyl, C₁₋₆alkoxy, and haloC₁₋₆alkoxy;
R¹³ and R¹⁴ are each independently selected from the group consisting of hydrogen, C₁₋₆alkyl, C₆₋₁₀aryl, and C₃₋₆cycloalkyl, wherein each C₁₋₆alkyl, C₆₋₁₀aryl, or C₃₋₆cycloalkyl may be optionally substituted by one or more substituents independently selected from the group consisting of halo, C₁₋₆alkyl, haloC₁₋₆alkyl, C₁₋₆alkoxy, and haloC₁₋₆alkoxy;
R^{a} and R^{b} are each independently hydrogen, C₁₋₆alkyl or C₆₋₁₀aryl;
wherein said desulfinylation step is performed by a continuous flow process.

2. A process for the preparation of a compound of formula (I), a stereoisomer, a hydrate or a solvate thereof,

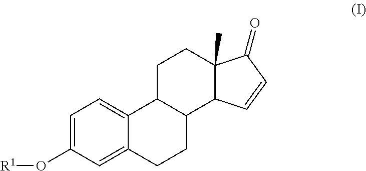

comprising the step of desulfinylation of a compound of formula (II) to produce a compound of formula (I);

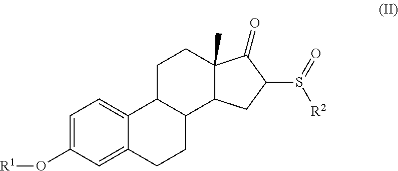

wherein:
R¹ is selected from the group comprising hydrogen, R³Si(R⁴)(R⁵)—, C₁₋₆alkyl, C₆₋₁₀aryl, C₆₋₁₀arylC₁₋₆alkyl, —CH₂—CH═CR^{a}R^{b}, C₁₋₆alkoxy, C₃₋₆cycloalkyl, and R⁶CO—; each of said C₁₋₆alkyl, C₆₋₁₀aryl, C₆₋₁₀arylC₁₋₆alkyl, and C₃₋₆cycloalkyl may be optionally substituted with one or more substituents each independently selected from the group consisting of halo, C₁₋₆alkyl, haloC₁₋₆alkyl, C₁₋₆alkoxy, haloC₁₋₆alkoxy, C₆₋₁₀aryl, heterocyclyl, and nitro;
R² is C₆₋₁₀aryl, or heteroaryl; wherein each C₆₋₁₀aryl or heteroaryl may be optionally substituted with one or more substituents each independently selected from the group consisting of halo, C₁₋₆alkyl, haloC₁₋₆alkyl, C₁₋₆alkoxy, haloC₁₋₆alkoxy, C₂₋₆alkenyl, C₂₋₆alkynyl, C₆₋₁₀aryl, heterocyclyl, heteroaryl, hydroxyl, —S(O)₂R⁷, —S(O)R⁸, CO₂R⁹, C(O)R¹⁰, —SR¹³, —C(O)SR¹⁴, NR¹¹R¹², cyano and nitro; preferably selected from the group consisting of fluoro, chloro, bromo, nitro, C₁₋₄alkyl, C₁₋₄alkyloxy, haloC₁₋₄alkyloxy;
R³, R⁴ and R⁵ are each independently C₁₋₆alkyl or C₆₋₁₀aryl, wherein each C₁₋₆alkyl or C₆₋₁₀aryl may be optionally substituted with one or more substituents each independently selected from the group consisting of halo, C₁₋₆alkyl, haloC₁₋₆alkyl, C₁₋₆alkoxy, and haloC₁₋₆alkoxy; preferably fluoro, chloro or C₁₋₄alkyl;
R⁶ is C₁₋₆alkyl, C₆₋₁₀aryl, or C₃₋₆cycloalkyl, wherein each C₁₋₆alkyl, C₆₋₁₀aryl, or C₃₋₆cycloalkyl may be optionally substituted by one or more substituents independently selected from the group consisting of halo, C₁₋₆alkyl, haloC₁₋₆alkyl, C₁₋₆alkoxy, and haloC₁₋₆alkoxy; preferably fluoro, chloro or C₁₋₄alkyl;
R⁷, R⁸, R⁹ and R¹⁰ are each independently selected from the group consisting of hydrogen, hydroxyl, C₁₋₆alkyl, C₆₋₁₀aryl, and C₃₋₆cycloalkyl, wherein each C₁₋₆alkyl, C₆₋₁₀aryl, or C₃₋₆cycloalkyl may be optionally substituted by one or more substituents independently selected from the group consisting of halo, C₁₋₆alkyl, haloC₁₋₆alkyl, C₁₋₆alkoxy, and haloC₁₋₆alkoxy;
R¹¹ and R¹² are each independently selected from the group consisting of hydrogen, C₁₋₆alkyl, C₆₋₁₀aryl, C₃₋₆cycloalkyl, wherein each C₁₋₆alkyl, C₆₋₁₀aryl, or C₃₋₆cycloalkyl may be optionally substituted by one or more substituents independently selected from the group consisting of halo, C₁₋₆alkyl, haloC₁₋₆alkyl, C₁₋₆alkoxy, and haloC₁₋₆alkoxy;
R¹³ and R¹⁴ are each independently selected from the group consisting of hydrogen, C₁₋₆alkyl, C₆₋₁₀aryl, and C₃₋₆cycloalkyl, wherein each C₁₋₆alkyl, C₆₋₁₀aryl, or C₃₋₆cycloalkyl may be optionally substituted by one or more substituents independently selected from the group consisting of halo, C₁₋₆alkyl, haloC₁₋₆alkyl, C₁₋₆alkoxy, and haloC₁₋₆alkoxy;
R^{a} and R^{b} are each independently hydrogen, C₁₋₆alkyl or C₆₋₁₀aryl;
wherein said desulfinylation step is performed by a continuous flow process.

3. A process for the preparation of a compound of formula (I), a stereoisomer, a hydrate or a solvate thereof,

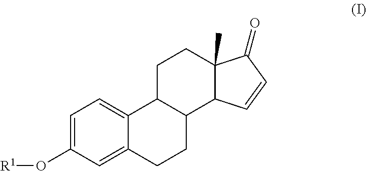

comprising the step of desulfinylation of a compound of formula (II) to produce compound of formula (I);

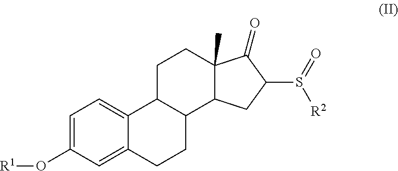

wherein:
R¹ is selected from the group comprising hydrogen, R³Si(R⁴)(R⁵)—, C₁₋₆alkyl, C₆₋₁₀aryl, C₆₋₁₀arylC₁₋₆alkyl, —CH₂—CH═CR^{a}R^{b}, and R⁶CO—; each of said C₁₋₆alkyl, C₆₋₁₀aryl, and C₆₋₁₀arylC₁₋₆alkyl may be optionally substituted with one or more substituents each independently selected from the group consisting of halo, C₁₋₆alkyl, haloC₁₋₆alkyl, C₁₋₆alkoxy, haloC₁₋₆alkoxy, C₆₋₁₀aryl, heterocyclyl, and nitro;
R² is C₆₋₁₀aryl, or heteroaryl; wherein each C₆₋₁₀aryl or heteroaryl may be optionally substituted with one or more substituents each independently selected from the group consisting of halo, C₁₋₆alkyl, haloC₁₋₆alkyl, C₁₋₆alkoxy, haloC₁₋₆alkoxy, and nitro;
R³, R⁴ and R⁵ are each independently C₁₋₆alkyl or C₆₋₁₀aryl, wherein each C₁₋₆alkyl or C₆₋₁₀aryl may be optionally substituted with one or more substituents each independently selected from the group consisting of halo, C₁₋₆alkyl, haloC₁₋₆alkyl, C₁₋₆alkoxy, and haloC₁₋₆alkoxy; preferably fluoro, chloro or C₁₋₄alkyl;
R⁶ is C₁₋₆alkyl, C₆₋₁₀aryl, or C₃₋₆cycloalkyl, wherein each C₁₋₆alkyl, C₆₋₁₀aryl, or C₃₋₆cycloalkyl may be optionally substituted by one or more substituents independently selected from the group consisting of halo, C₁₋₆alkyl, haloC₁₋₆alkyl, C₁₋₆alkoxy, and haloC₁₋₆alkoxy;
R^{a} and R^{b} are each independently hydrogen, C₁₋₆alkyl or C₆₋₁₀aryl;
wherein said desulfinylation step is performed by a continuous flow process.

4. The process according to statements 1 to 3, wherein said desulfinylation step is performed by thermolysis.

5. The process according to any one of statements 1 to 4, wherein said desulfinylation step is performed by thermolysis, at a thermolysis temperature, T, of at least 100° C.; preferably at a temperature of at least 110° C., preferably at a temperature of at least 120° C.; preferably at a temperature of at least 130° C.; preferably at a temperature of at least 140° C.; preferably at a temperature of at least 150° C.; preferably at a temperature of at least 160° C.; preferably at a temperature of at least 170° C.; preferably at a temperature of at least 175° C.; preferably at a temperature of at least 180° C.

6. The process according to any one of statements 1 to 5, wherein said desulfinylation step is performed by thermolysis, at a thermolysis temperature, T, of at least 100° C. to at most 300° C.; preferably at a temperature of at least 120° C. to at most 300° C.; preferably at a temperature of at least 140° C. to at most 300° C.; preferably at a temperature of at least 160° C. to at most 300° C.; preferably at a temperature of at least 170° C. to at most 290° C.; preferably at a temperature of at least 175° C. to at most 280° C.; preferably at a temperature of at least 175° C. to at most 270° C.; preferably at a temperature of at least 175° C. to at most 260° C.; preferably at a temperature of at least 175° C. to at most 250° C.; preferably at a temperature of at least 175° C. to at most 240° C.; preferably at a temperature of at least 175° C. to at most 230° C.; preferably at a temperature of at least 180° C. to at most 230° C.; preferably at a temperature of at least 180° to at most 220° C.; preferably at a temperature of at least 190° to at most 220° C.; preferably at a temperature of at least 200° to at most 220° C.; preferably at a temperature of at least 190° C. to at most 215° C.; preferably at a temperature of at least 205° C. to at most 215° C.

7. The process according to any one of statements 1 to 6, wherein said desulfinylation step is performed by thermolysis, wherein said thermolysis is performed at a pressure, P, of at least 1 bar, preferably at a pressure of at least 2.0 bar; preferably at a pressure of at least 3.0 bar; preferably at a pressure of at least 4.0 bar; preferably at a pressure of at least 5.0 bar.

8. The process according to any one of statements 1 to 7, wherein said desulfinylation step is performed by thermolysis, wherein said thermolysis is performed at a pressure, P, of at least 1.0 bar to at most 60.0 bar; preferably at least 3.0 bar to at most 60.0 bar; preferably at a pressure of at least 5.0 bar to at most 60.0 bar; preferably at a pressure of at least 10.0 to at most 55.0 bar; preferably at a pressure of at least 12.0 to at most 50.0 bar; preferably at a pressure of at least 20.0 to at most 50.0 bar; preferably at a pressure of at least 25.0 to at most 45.0; preferably at a pressure of at least 30.0 to at most 40.0 bar; preferably at a pressure of at least 12.0 to at most 40.0 bar; preferably at a pressure of at least 12.5 to at most 35.0 bar; preferably at a pressure of at least 15.0 to at most 35.0 bar.

9. The process according to any one of statements 1 to 8, wherein said desulfinylation step is performed by thermolysis, for a thermolysis time, t, of at most 60 min; preferably of at most 40 min, preferably of at most 30 min; preferably at most 25 min; preferably at most 20 min; preferably at most 18 min.

10. The process according to any one of statements 1 to 9, wherein said desulfinylation step is performed by thermolysis, for a thermolysis time, t, of at least 0.1 min, preferably of at least 0.3 min; preferably at least 0.5 min; preferably at least 0.7 min; preferably at least 0.9 min; preferably at least 1.0 min.

11. The process according to any one of statements 1 to 10, wherein said desulfinylation step is performed by thermolysis, for a thermolysis time, t, of at least 0.1 min to at most 30 min; preferably at least 0.5 min to at most 30 min; preferably at least 1.0 min to at most 25 min; preferably at least 1.0 min to at most 20 min, preferably at least 1.0 min to at most 15 min; preferably at least 2 min to at most 10 min, preferably at least 2 min to at most 8 min; preferably at least 3 min to at most 8 min; preferably at least 3 min to at most 6 min; preferably at least 3 min to at most 10 min, preferably at least 4 min to at most 8 min; preferably at least 5 min to at most 8 min.

12. The process according to any one of statements 1 to 11, wherein the desulfinylation step is performed in at least one diluent; preferably performed in solution; preferably performed using at least one solution.

13. The process according to any one of statements 1 to 12, wherein the desulfinylation step is performed in at least one diluent and compound of formula (II) is present in the at least one diluent in an amount of at least 0.01 mole/L of diluent; preferably of at least 0.02 mole/L; preferably of at least 0.05 mole/L; preferably of at least 0.10 mole/L of diluent.

14. The process according to any one of statements 1 to 13, wherein the desulfinylation step is performed in at least one diluent and compound of formula (II) is present in the at least one diluent in an amount of at most 5.0 mole/L of diluent; preferably of at most 4.0 mole/L; preferably of at most 3.0 mole/L; preferably of at most 2.0 mole/L; preferably of at most 1.0 mole/L of diluent.

15. The process according to any one of statements 1 to 14, wherein the desulfinylation step is performed in at least one diluent and compound of formula (II) is present in the at least one diluent in an amount of at least 0.01 mole/L to at most 5.0 mole/L of diluent; preferably of at least 0.01 mole/L to at most 3.0 mole/L; preferably of at least 0.01 mole/L to at most 1.0 mole/L; preferably of at least 0.01 mole/L to at most 0.80 mole/L; preferably at least 0.02 mole/L to at most 0.70 mole/L; preferably at least 0.05 mole/L to at most 0.60 mole/L; preferably at least 0.06 mole/L to at most 0.56 mole/L; preferably at least 0.10 mole/L to at most 0.55 mole/L; preferably at least 0.15 mole/L to at most 0.50 mole/L; preferably at least 0.15 mole/L to at most 0.45 mole/L; preferably at least 0.15 mole/L to at most 0.40 mole/L; preferably at least 0.15 mole/L to at most 0.35 mole/L; preferably at least 0.15 mole/L to at most 0.30 mole/L; preferably at least 0.15 mole/L to at most 0.25 mole/L of diluent.

16. The process according to any one of statements 1 to 15, wherein the desulfinylation step is performed in at least one diluent and compound of formula (II) is present in the at least one diluent in an amount of at least 0.01 mole/L to at most 5.0 mole/L; preferably of at least 0.01 mole/L to at most 4.0 mole/L; preferably of at least 0.01 mole/L to at most 2.0 mole/L; preferably of at least 0.01 mole/L to at most 0.90 mole/L; preferably at least 0.02 mole/L to at most 0.80 mole/L; preferably at least 0.05 mole/L to at most 0.70 mole/L; preferably at least 0.06 mole/L to at most 0.56 mole/L; preferably at least 0.06 mole/L to at most 0.40 mole/L; preferably at least 0.06 mole/L to at most 0.30 mole/L; preferably at least 0.06 mole/L to at most 0.25 mole/L of diluent

17. The process according to any one of statements 1 to 16, wherein the desulfinylation step is performed in at least one diluent and compound of formula (II) is present in the at least one diluent in the form of a solution of the said at least one diluent.

18. The process according to any one of statements 1 to 17, wherein:
R¹ is R³Si(R⁴)(R⁵)—;
R² is C₆₋₁₀aryl, or heteroaryl; wherein each C₆₋₁₀aryl or heteroaryl may be optionally substituted with one or more substituents each independently selected from the group consisting of halo, C₁₋₆alkyl, haloC₁₋₆alkyl, C₁₋₆alkoxy, haloC₁₋₆alkoxy, and nitro;
preferably from the group consisting of fluoro, chloro, bromo, nitro, C₁₋₄alkyl, C₁₋₄alkyloxy, and haloC₁₋₄alkyloxy; preferably, R² is C₆₋₁₀aryl, or 5-6 membered heteroaryl containing at least one N and/or S; wherein each C₆₋₁₀aryl or heteroaryl may be optionally substituted with one or more substituents each independently selected from the group consisting of halo, C₁₋₆alkyl, haloC₁₋₆alkyl, C₁₋₆alkoxy, haloC₁₋₆alkoxy, and nitro; preferably from the group consisting of fluoro, chloro, bromo, nitro, C₁₋₄alkyl, C₁₋₄alkyloxy, and haloC₁₋₄alkyloxy; preferably, R² is C₆₋₁₀aryl, or 5-6 membered heteroaryl containing at least one N; wherein each C₆₋₁₀aryl or heteroaryl may be optionally substituted with one or more substituents each independently selected from the group consisting of halo, C₁₋₆alkyl, haloC₁₋₆alkyl, C₁₋₆alkoxy, haloC₁₋₆alkoxy, and nitro; preferably from the group consisting of fluoro, chloro, bromo, nitro, C₁₋₄alkyl, C₁₋₄alkyloxy, and haloC₁₋₄alkyloxy; preferably R² is phenyl, or pyridinyl; wherein each phenyl or pyridinyl may be optionally substituted with one or more substituents each independently selected from the group consisting of halo, C₁₋₆alkyl, haloC₁₋₆alkyl, C₁₋₆alkoxy, halo_{C1-6}alkoxy, and nitro; preferably from the group consisting of fluoro, chloro, bromo, nitro, C₁₋₄alkyl, C₁₋₄alkyloxy, and haloC₁₋₄alkyloxy; and
R³, R⁴ and R⁵ are each independently C₁₋₆alkyl or C₆₋₁₀aryl, wherein each C₁₋₆alkyl or C₆₋₁₀aryl may be optionally substituted with one or more substituents each independently selected from the group consisting of halo, C₁₋₆alkyl, haloC₁₋₆alkyl, C₁₋₆alkoxy, and haloC₁₋₆alkoxy; preferably fluoro, chloro or C₁₋₄alkyl; preferably R³, R⁴ and R⁵ are each independently C₁₋₆alkyl or phenyl, wherein each C₁₋₆alkyl or phenyl may be optionally substituted with one or more substituents each independently selected from the group consisting of halo, C₁₋₆alkyl, haloC₁₋₆alkyl, C₁₋₆alkoxy, and haloC₁₋₆alkoxy; preferably fluoro, chloro or C₁₋₄alkyl.

19. The process according to any one of statements 1 to 17, wherein:
R¹ is R³Si(R⁴)(R⁵)—;
R² is C₆₋₁₀aryl, or heteroaryl; wherein each C₆₋₁₀aryl or heteroaryl may be optionally substituted with one or more substituents each independently selected from the group consisting of halo, C₁₋₆alkyl, haloC₁₋₆alkyl, C₁₋₆alkoxy, haloC₁₋₆alkoxy, C₂₋₆alkenyl, C₂₋₆alkynyl, C₆₋₁₀aryl, heterocyclyl, heteroaryl, hydroxyl, —S(O)₂R⁷, —S(O)R⁸, CO₂R⁹, C(O)R¹⁰, —SR¹³, —C(O)SR¹⁴, NR¹¹R¹², cyano and nitro; preferably from the group consisting of fluoro, chloro, bromo, nitro, C₁₋₄alkyl, C₁₋₄alkyloxy, haloC₁₋₄alkyloxy, C₂₋₄alkenyl, C₂₋₄alkynyl, phenyl, heterocyclyl, heteroaryl, —S(O)₂R⁷, CO₂R⁹, C(O)R¹⁰, thiol, C₁₋₆alkylthio, —C(O)SR¹⁴, amino, mono-C₁₋₄alkylamino, di-C₁₋₄alkylamino, cyano;
preferably, R² is C₆₋₁₀aryl, or 5-6 membered heteroaryl containing at least one N and/or S; wherein each C₆₋₁₀aryl or heteroaryl may be optionally substituted with one or more substituents each independently selected from the group consisting of halo, C₁₋₆alkyl, haloC₁₋₆alkyl, C₁₋₆alkoxy, haloC₁₋₆alkoxy, C₂₋₆alkenyl, C₂₋₆alkynyl, C₆₋₁₀aryl, heterocyclyl, heteroaryl, hydroxyl, —S(O)₂R⁷, —S(O)R⁸, CO₂R⁹, C(O)R¹⁰, —SR¹³, —C(O)SR¹⁴, NR¹¹R¹², cyano and nitro; preferably from the group consisting of fluoro, chloro, bromo, nitro, C₁₋₄alkyl, C₁₋₄alkyloxy, haloC₁₋₄alkyloxy, C₂₋₄alkenyl, C₂₋₄alkynyl, phenyl, heterocyclyl, heteroaryl, —S(O)₂R⁷, —CO₂R⁹, —C(O)R¹⁰, thiol, C₁₋₆alkylthio, —C(O)SR¹⁴, amino, mono-C₁₋₄alkylamino, di-C₁₋₄alkylamino, cyano; preferably, R² is C₆₋₁₀aryl, or 5-6 membered heteroaryl containing at least one N; wherein each C₆₋₁₀aryl or heteroaryl may be optionally substituted with one or more substituents each independently selected from the group consisting of halo, C₁₋₆alkyl, haloC₁₋₆alkyl, C₁₋₆alkoxy, haloC₁₋₆alkoxy, C₂₋₆alkenyl, C₂₋₆alkynyl, C₆₋₁₀aryl, heterocyclyl, heteroaryl, hydroxyl, —S(O)₂R⁷, —S(O)R⁸, —CO₂R⁹, —C(O)R¹⁰, —SR¹³, —C(O)SR¹⁴, NR¹¹R¹², cyano and nitro; preferably from the group consisting of fluoro, chloro, bromo, nitro, C₁₋₄alkyl, C₁₋₄alkyloxy, haloC₁₋₄alkyloxy, C₂₋₄alkenyl, C₂₋₄alkynyl, phenyl, heterocyclyl, heteroaryl, —S(O)₂R⁷, —CO₂R⁹, —C(O)R¹⁰, thiol, C₁₋₆alkylthio, —C(O)SR¹⁴, amino, mono-C₁₋₄alkylamino, di-C₁₋₄alkylamino, cyano; preferably R² is phenyl, or pyridinyl; wherein each phenyl or pyridinyl may be optionally substituted with one or more substituents each independently selected from the group consisting of halo, C₁₋₆alkyl, haloC₁₋₆alkyl, C₁₋₆alkoxy, haloC₁₋₆alkoxy, C₂₋₆alkenyl, C₂₋₆alkynyl, C₆₋₁₀aryl, heterocyclyl, heteroaryl, hydroxyl, —S(O)₂R⁷, —S(O)R⁸, —CO₂R⁹, —C(O)R¹⁰, —SR¹³, —C(O)SR¹⁴, NR¹¹R¹², cyano and nitro; preferably from the group consisting of fluoro, chloro, bromo, nitro, C₁₋₄alkyl, C₁₋₄alkyloxy, haloC₁₋₄alkyloxy, C₂₋₄alkenyl, C₂₋₄alkynyl, phenyl, heterocyclyl, heteroaryl, —S(O)₂R⁷, —CO₂R⁹, —C(O)R¹⁰, thiol, C₁₋₆alkylthio, —C(O)SR¹⁴, amino, mono-C₁₋₄alkylamino, di-C₁₋₄alkylamino, cyano;
R³, R⁴ and R⁵ are each independently C₁₋₆alkyl or C₆₋₁₀aryl, wherein each C₁₋₆alkyl or C₆₋₁₀aryl may be optionally substituted with one or more substituents each independently selected from the group consisting of halo, C₁₋₆alkyl, haloC₁₋₆alkyl, C₁₋₆alkoxy, and haloC₁₋₆alkoxy; preferably fluoro, chloro or C₁₋₄alkyl; preferably R³, R⁴ and R⁵ are each independently C₁₋₆alkyl or phenyl, wherein each C₁₋₆alkyl or phenyl may be optionally substituted with one or more substituents each independently selected from the group consisting of halo, C₁₋₆alkyl, haloC₁₋₆alkyl, C₁₋₆alkoxy, and haloC₁₋₆alkoxy; preferably fluoro, chloro or C₁₋₄alkyl; and
R⁷, R⁸, R⁹ and R¹⁰ are each independently selected from the group consisting of hydrogen, hydroxyl, C₁₆alkyl, C₆₁₀aryl, and C₃₋₆cycloalkyl, wherein each C₁₋₆alkyl, C₆₋₁₀aryl, or C₃₋₆cycloalkyl may be optionally substituted by one or more substituents independently selected from the group consisting of halo, C₁₋₆alkyl, haloC₁₋₆alkyl, C₁₋₆alkoxy, and haloC₁₋₆alkoxy; preferably R⁷, R⁸, R⁹ and R¹⁰ are each independently hydrogen, C₁₋₄alkyl, phenyl, and C₃₋₆cycloalkyl wherein each C₁₋₄alkyl, phenyl or C₃₋₆cycloalkyl may be optionally substituted by one or more substituents independently selected from the group consisting of halo, C₁₋₆alkyl, haloC₁₋₆alkyl, C₁₋₆alkoxy, and haloC₁₋₆alkoxy; preferably from the group consisting of fluoro, chloro, bromo, C₁₋₄alkyl, C₁₋₄alkyloxy, and haloC₁₋₄alkyloxy;
R¹¹ and R¹² are each independently selected from the group consisting of hydrogen, C₁₋₆alkyl, C₆₋₁₀aryl, C₃₋₆cycloalkyl; wherein each C₁₋₆alkyl, C₆₋₁₀aryl, or C₃₋₆cycloalkyl may be optionally substituted by one or more substituents independently selected from the group consisting of halo, C₁₋₆alkyl, haloC₁₋₆alkyl, C₁₋₆alkoxy, and haloC₁₋₆alkoxy; preferably R¹¹ and R¹² are each independently hydrogen, C₁₋₄alkyl, phenyl, and C₃₋₆cycloalkyl wherein each C₁₋₄alkyl, phenyl or C₃₋₆cycloalkyl may be optionally substituted by one or more substituents independently selected from the group consisting of halo, C₁₋₆alkyl, haloC₁₋₆alkyl, C₁₋₆alkoxy, and haloC₁₋₆alkoxy; preferably from the group consisting of fluoro, chloro, bromo, C₁₋₄alkyl, C₁₋₄alkyloxy, and haloC₁₋₄alkyloxy;
R¹³ and R¹⁴ are each independently selected from the group consisting of hydrogen, C₁₋₆alkyl, C₆₋₁₀aryl, and C₃₋₆cycloalkyl, wherein each C₁₋₆alkyl, C₆₋₁₀aryl, or C₃₋₆cycloalkyl may be optionally substituted by one or more substituents independently selected from the group consisting of halo, C₁₋₆alkyl, haloC₁₋₆alkyl, C₁₋₆alkoxy, and haloC₁₋₆alkoxy; preferably R¹³ and R¹⁴ are each independently hydrogen, C₁₋₄alkyl, phenyl, and C₃₋₆cycloalkyl wherein each C₁₋₄alkyl, phenyl or C₃₋₆cycloalkyl may be optionally substituted by one or more substituents independently selected from the group consisting of halo, C₁₋₆alkyl, haloC₁₋₆alkyl, C₁₋₆alkoxy, and haloC₁₋₆alkoxy; preferably from the group consisting of fluoro, chloro, bromo, C₁₋₄alkyl, C₁₋₄alkyloxy, and haloC₁₋₄alkyloxy.

20. The process according to any one of statements 1 to 19, comprising the following steps:
(a) forming a mixture by contacting a compound of formula (II) with:
   at least one diluent;
   optionally at least one scavenger; and
   optionally at least one base (organic or inorganic), preferably at least one organic base; or
   optionally no base is used;
(b) feeding the mixture to an inlet of a channel of a reaction zone,
(c) driving a flow of the mixture along the channel from the inlet to an outlet, to form reaction products including at least compound of formula (I);
(d) recovering the reaction products at the outlet and
(e) optionally separating the at least one compound of formula (I) from the other reaction products or keeping the compound of formula (I) in the reaction products.

21. The process according to any one of statements 1 to 20, comprising the following steps:
(a) forming a solution by contacting a compound of formula (II) with:
   at least one solvent;
   optionally at least one scavenger; and
   optionally at least one base (organic or inorganic), preferably at least one organic base, or
   optionally no base is used;
(b) feeding the solution to an inlet of a channel of a reaction zone,
(c) driving a flow of the solution along the channel from the inlet to an outlet, to form reaction products including at least compound of formula (I);
(d) recovering the reaction products at the outlet and
(e) optionally separating the at least one compound of formula (I) from the other reaction products or keeping the compound of formula (I) in the reaction products.

22. The process according to statements 20 or 21, further comprising the steps of
(d1) feeding the reaction products from step (d) to an inlet of a channel of a cooling zone,
(d2) exposing the reaction products from step (d) to cooling by driving a flow of the reaction products along the channel from the inlet to an outlet, for a cooling time, tc, and at a cooling temperature, Tc; and
(d3) recovering the cooled reaction products at the outlet of the cooling zone.

23. The process according to any one of statements 20 to 22, wherein the reaction zone is a thermolysis reaction zone and step (c) comprises exposing the mixture to thermolysis by driving a flow of the mixture along the channel from the inlet to the outlet, for a thermolysis time, t, at a pressure, P, and at a thermolysis temperature, T, to form thermolysis reaction products including at least compound of formula (I).

24. The process according to anyone of statements 20 to 23, wherein the reaction of step (c) is carried out at a pressure, P, of at least 1.0 bar, preferably at least 2.0 bar; preferably at least 3.0 bar; preferably at least 4.0 bar; preferably at least 5.0 bar; preferably said reaction of step (c) is a thermolysis reaction carried out at a pressure, P, as described herein above.

25. The process according to any one of statements 20 to 24, wherein the reaction of step (c) is carried out at a pressure, P, of at least 1.0 bar to at most 60.0 bar; preferably at least 3.0 bar to at most 60.0 bar; preferably at least 5.0 bar to at most 60.0 bar; preferably at a pressure of at least 10.0 to at most 55.0 bar; preferably at a pressure of at least 20.0 to at most 50.0 bar; preferably at a pressure of at least 30.0 to at most 40.0, preferably at a pressure of at least 10.0 to at most 40.0 bar, preferably a ta pressure of at least 10 to at most 30.0 bar, preferably at a pressure of at least 12.0 to at most 50.0 bar; preferably at a pressure of at least 12.0 to at most 40.0 bar; preferably at a pressure of at least 11.0 to at most 35.0 bar; preferably said reaction of step (c) is a thermolysis reaction carried out at a pressure, P, as described herein above.

26. The process according to any one of statements 20 to 25, wherein the reaction of step (c) is carried for a reaction time, t, of at most 60 min; preferably of at most 40 min, preferably of at most 30 min; preferably at most 25 min; preferably at most 20 min; preferably at most 18 min; preferably said reaction of step (c) is a thermolysis reaction carried out for a thermolysis time, t, as described herein above.

27. The process according to any one of statements 20 to 26, wherein the reaction of step (c) is carried for a reaction time, t, of at least 0.1 min; preferably at least 0.3 min; preferably at least 0.5 min; preferably at least 0.7 min; preferably at least 0.9 min; preferably at least 1.0 min; preferably said reaction of step (c) is a thermolysis reaction carried out for a thermolysis time, t, as described herein above

28. The process according to any one of statements 20 to 27, wherein the reaction of step (c) is carried for a reaction time, t, of at least 0.1 min to at most 30 min; preferably at least 0.3 min to at most 30 min; preferably at least 0.5 min to at most 30 min; preferably at least 1.0 min to at most 25 min; preferably at least 1.0 min to at most 20 min, preferably at least 1.0 min to at most 15 min; preferably at least 1.5 min to at most 10 min, preferably at least 1.5 min to at most 8 min; preferably at least 2.5 min to at most 8 min; preferably at least 2.0 min to at most 6 min; preferably at least 2.5 min to at most 10 min, preferably at least 4.0 min to at most 8.0 min, preferably at least 5.0 min to at most 8.0 min, preferably said reaction of step (c) is a thermolysis reaction carried out for a thermolysis time, t, as described herein above.

29. The process according to any one of statements 20 to 28, wherein the reaction of step (c) is carried at a reaction temperature, T, of at least 100° C.; preferably at a temperature of at least 110° C., preferably at a temperature of at least 120° C.; preferably at a temperature of at least 130° C.; preferably at a temperature of at least 140° C.; preferably at a temperature of at least 150° C.; preferably at a temperature of at least 160° C.; preferably at a temperature of at least 170° C.; preferably at a temperature of at least 175° C.; preferably at a temperature of at least 180° C.; preferably said reaction of step (c) is a thermolysis reaction carried out at a thermolysis temperature, T, as described herein above.

30. The process according to any one of statements 20 to 29, wherein the reaction of step (c) is carried at a reaction temperature, T, of at least 100° C. to at most 300° C.; preferably at a temperature of at least 120° C. to at most 300° C.; preferably at a temperature of at least 140° C. to at most 300° C.; preferably at a temperature of at least 160° C. to at most 300° C.; preferably at a temperature of at least 170° C. to at most 290° C.; preferably at a temperature of at least 175° C. to at most 280° C.; preferably at a temperature of at least 175° C. to at most 270° C.; preferably at a temperature of at least 175° C. to at most 260° C.; preferably at a temperature of at least 175° C. to at most 250° C.; preferably at a temperature of at least 175° C. to at most 240° C.; preferably at a temperature of at least 175° C. to at most 230° C.; preferably at a temperature of at least 180° C. to at most 230° C.; preferably at a temperature of at least 180° to at most 220° C.; preferably at a temperature of at least 190° to at most 220° C.; preferably at a temperature of at least 200° to at most 220° C.; preferably at a temperature of at least 190° C. to at most 215° C.; preferably at a temperature of at least 205° C. to at most 215° C.; preferably said reaction of step (c) is a thermolysis reaction carried out at a thermolysis temperature, T, as described herein above.

31. The process according to any one of statements 20 to 30, wherein the cooling temperature, Tc, of step (d2) is of at least −10.0° C. to at most 20.0° C., preferably of at least −5.0° C. to at most 20.0° C., preferably of at least −5.0° C. to at most 15.0° C., preferably of at least −5.0° C. to at most 10.0° C.; preferably at a temperature of at least −4.0° C. to at most 8.0° C.; preferably at a temperature of at least −3.0° C. to at most 7.0° C.; preferably at a temperature of at least −2.0° C. to at most 6.0° C.; preferably at a temperature of at least −1.0° C. to at most 5.0° C.

32. The process according to any one of statements 20 to 31, wherein the cooling is carried out at the same pressure as reaction step (c).

33. The process according to any one of statements 20 to 32, wherein the cooling is carried out at atmospheric pressure.

34. The process according to any one of statements 20 to 33, wherein the cooling time, tc, of step (d2) is of at most 120 min; preferably of at most 60 min, preferably at most 30 min, preferably at most 25 min; preferably at most 15 min, preferably at most 10 min; preferably at most 5 min; preferably at most 3 min; preferably at most 1 min.

35. The process according to any one of statements 20 to 34, wherein the cooling time, tc, of step (d2) is of at least 0.08 min to at most 20 min; preferably at least 0.08 min to at most 15 min; preferably at least 0.08 min to at most 10 min; preferably at least 0.08 min to at most 5 min; preferably at least 0.1 min to at most 4 min; preferably at least 0.1 min to at most 3 min; preferably at least 0.4 min to at most 3 min; preferably at least 0.4 min to at most 2 min; preferably at least 0.4 min to at most 1.5 min; preferably at least 0.4 min to at most 1.2 min; preferably at least 0.5 min to at most 1.2 min.

36. The process according to any one of statements 20 to 35, wherein compound of formula (II) is present in the mixture of step (a) in an amount of at least 0.01 mole/L of diluent; preferably of at least 0.02 mole/L; preferably of at least 0.05 mole/L; preferably of at least 0.10 mole/L; preferably of at most 5.0 mole/L; preferably of at most 4.0 mole/L; preferably of at most 3.0 mole/L; preferably of at most 2.0 mole/L; preferably of at most 1.0 mole/L of diluent.

37. The process according to any one of statements 20 to 36, wherein compound of formula (II) is present in the mixture of step (a) in an amount of at least 0.01 mole/L to at most 5.0 mole/L of diluent; preferably of at least 0.01 mole/L to at most 3.0 mole/L; preferably of at least 0.01 mole/L to at most 1.0 mole/L; preferably of at least 0.01 mole/L to at most 0.80 mole/L; preferably at least 0.02 mole/L to at most 0.70 mole/L; preferably at least 0.05 mole/L to at most 0.60 mole/L; preferably at least 0.06 mole/L to at most 0.56 mole/L; preferably at least 0.10 mole/L to at most 0.55 mole/L; preferably at least 0.15 mole/L to at most 0.50 mole/L; preferably at least 0.15 mole/L to at most 0.45 mole/L; preferably at least 0.15 mole/L to at most 0.40 mole/L; preferably at least 0.15 mole/L to at most 0.35 mole/L; preferably at least 0.15 mole/L to at most 0.30 mole/L; preferably at least 0.15 mole/L to at most 0.25 mole/L of diluent.

38. The process according to any one of statements 20 to 37, wherein compound of formula (II) is present in the mixture of step (a) in an amount of at least 0.01 mole/L to at most 5.0 mole/L of diluent; preferably of at least 0.01 mole/L to at most 4.0 mole/L; preferably of at least 0.01 mole/L to at most 2.0 mole/L; preferably of at least 0.01 mole/L to at most 0.90 mole/L; preferably at least 0.02 mole/L to at most 0.60 mole/L; preferably at least 0.06 mole/L to at most 0.50 mole/L; preferably at least 0.06 mole/L to at most 0.40 mole/L; preferably at least 0.06 mole/L to at most 0.30 mole/L; preferably at least 0.06 mole/L to at most 0.25 mole/L of diluent.

39. The process according to any one of statements 21 to 38, wherein compound of formula (II) is present in the solution of step (a) in an amount of at least 0.01 mole/L of solution; preferably of at least 0.02 mole/L; preferably of at least 0.05 mole/L; preferably of at least 0.10 mole/L; preferably of at most 5.0 mole/L; preferably of at most 4.0 mole/L; preferably of at most 3.0 mole/L; preferably of at most 2.0 mole/L; preferably of at most 1.0 mole/L of solution.

40. The process according to any one of statements 21 to 36, 39, wherein compound of formula (II) is present in the solution of step (a) in an amount of at least 0.01 mole/L to at most 5.0 mole/L of solution; preferably of at least 0.01 mole/L to at most 3.0 mole/L; preferably of at least 0.01 mole/L to at most 1.0 mole/L; preferably of at least 0.01 mole/L to at most 0.80 mole/L; preferably at least 0.02 mole/L to at most 0.70 mole/L; preferably at least 0.05 mole/L to at most 0.60 mole/L; preferably at least 0.06 mole/L to at most 0.56 mole/L; preferably at least 0.10 mole/L to at most 0.55 mole/L; preferably at least 0.15 mole/L to at most 0.50 mole/L; preferably at least 0.15 mole/L to at most 0.45 mole/L; preferably at least 0.15 mole/L to at most 0.40 mole/L; preferably at least 0.15 mole/L to at most 0.35 mole/L; preferably at least 0.15 mole/L to at most 0.30 mole/L; preferably at least 0.15 mole/L to at most 0.25 mole/L of solution.

41. The process according to any one of statements 21 to 36, 39-40, wherein compound of formula (II) is present in the solution of step (a) in an amount of at least 0.01 mole/L to at most 5.0 mole/L of solution; preferably of at least 0.01 mole/L to at most 4.0 mole/L; preferably of at least 0.01 mole/L to at most 2.0 mole/L; preferably of at least 0.01 mole/L to at most 0.90 mole/L; preferably at least 0.02 mole/L to at most 0.60 mole/L; preferably at least 0.06 mole/L to at most 0.50 mole/L; preferably at least 0.06 mole/L to at most 0.40 mole/L; preferably at least 0.06 mole/L to at most 0.30 mole/L; preferably at least 0.06 mole/L to at most 0.25 mole/L of solution.

42. The process according to any one of statements 1 to 41, wherein the desulfinylation step is carried out in the presence of at least one scavenger.

43. The process according to any one of statements 1 to 42, wherein the desulfinylation step is carried out in the presence of at least one scavenger, wherein the at least one scavenger is selected from the group comprising dialkyl acetylenedicarboxylate (such as dimethyl acetylenedicarboxylate (DMAD) or diethyl acetylenedicarboxylate (DEtAD)), triethylamine, (2,2,6,6-tetramethylpiperidin-1-yl)oxyl (TEMPO), 2,6-lutidine, diethyl fumarate (DEF), 2-mercaptobenzothiazole (MBT), 1,4-dimethoxybenzene, crotonaldehyde, cyclohexenone, 1,4-diazabicyclo[2.2.2]octane (DABCO), diisopropyl azodicarboxylate (DIAD), methyl propiolate, phenylacetylene, N,N-diisopropylethylamine (DIPEA), trimethyl phosphite (TMP), methanol, dimethylsulfoxide, p-benzoquinone; NaHCO₃, K₂CO₃, Na₂CO₃, CaCO₃, Cs₂CO₃; and a combination thereof; preferably the at least one scavenger is dialkyl acetylenedicarboxylate; preferably the at least one scavenger is DMAD; preferably the at least one scavenger is TEMPO; preferably the at least one scavenger is DABCO.

44. The process according to any one of statements 1 to 43, wherein the desulfinylation step is carried out in the presence of at least one scavenger, and wherein the at least one scavenger is present in an amount of at least 0.1 to at most 10.0 equivalents; preferably in an amount of at least 0.2 to at most 9.0 equivalents; preferably in an amount of at least 0.3 to at most 8.5 equivalents; preferably in an amount of at least 0.2 to at most 8.0 equivalents; preferably in an amount of at least 0.5 to at most 7.0 equivalents; preferably in an amount of at least 0.5 to at most 5.0 equivalents; preferably 0.5 to at most 4.0 equivalents; preferably in an amount of at least 1.0 to at most 3.0 equivalents; preferably in an amount of at least 1.0 to at most 2.0 equivalents.

45. The process according to any one of statements 1 to 44, wherein the desulfinylation step is carried out in the presence of two or more scavengers, and wherein the scavengers are present in an amount of at least 0.1 to at most 10.0 equivalents; preferably in an amount of at least 0.2 to at most 9.0 equivalents; preferably in an amount of at least 0.3 to at most 8.5 equivalents; preferably in an amount of at least 0.2 to at most 8.0 equivalents; preferably in an amount of at least 0.5 to at most 7.0 equivalents; preferably in an amount of at least 0.5 to at most 5.0 equivalents; preferably 0.5 to at most 4.0 equivalents; preferably in an amount of at least 1.0 to at most 3.0 equivalents; preferably in an amount of at least 1.0 to at most 2.0 equivalents.

46. The process according to any one of statements 1 to 45, wherein the desulfinylation step is carried out in the absence of a base.

47. The process according to any one of statements 1 to 45, wherein the desulfinylation step is carried out in the presence of at least one base; preferably at least one organic base.

48. The process according to any one of statements 1 to 45, 47, wherein the desulfinylation step is carried out in the presence of at least one organic base, and wherein the at least one organic base is selected from the group comprising triethylamine, 1,8-diazabicyclo[5.4.0]undec-7-ene (DBU), 1,4-diazabicyclo[2.2.2]octane (DABCO), N,N-diisopropylethylamine (DIPEA), (2,2,6,6-tetramethylpiperidin-1-yl)oxyl (TEMPO), imidazole, 2,6-lutidine, and a combination thereof; preferably the at least one organic base is triethylamine.

49. The process according to any one of statements 1 to 45, 47, 48, wherein the desulfinylation step is carried out in the presence of at least one organic base, and wherein the at least one organic base is present in an amount of at least 0.01 to at most 10.0 equivalents; preferably at least 0.01 to at most 5.0 equivalents; preferably 0.01 to at most 1.0 equivalents; preferably at least 0.01 to at most 0.9 equivalents; preferably at least 0.05 to at most 0.8 equivalents; preferably at least 0.05 to at most 0.5 equivalents, preferably 0.05 to at most 0.3 equivalents; preferably at least 0.05 to 0.2 equivalents.

50. The process according to any one of statements 1 to 45, 47 to 49, wherein the desulfinylation step is carried out in the presence of two or more organic bases, and wherein the organic bases are present in combined amount of at least 0.01 to at most 10.0 equivalents; preferably at least 0.01 to at most 5.0 equivalents; preferably 0.01 to at most 1.0 equivalents; preferably at least 0.01 to at most 0.9 equivalents; preferably at least 0.05 to at most 0.8 equivalents; preferably at least 0.05 to at most 0.5 equivalents, preferably 0.05 to at most 0.3 equivalents; preferably at least 0.05 to 0.2 equivalents.

51. The process according to any one of statements 1 to 50, wherein the desulfinylation step is carried out in the presence of at least one scavenger and at least one organic base.

52. The process according to any one of statements 1 to 51, wherein the desulfinylation step is carried out in the presence of at least one diluent, preferably at least one solvent, preferably wherein the at least one solvent is selected from the group comprising toluene, o-xylene, m-xylene, p-xylene, 1,2-dichloroethane, chloroform, tetrachloromethane, tetrahydrofuran, 2-methyltetrahydrofuran, 1,4-dioxane, methyl tert-butyl ether, methyl acetate, ethyl acetate, butyl acetate, iso-butyl acetate, n-butyl acetate, anisole, chlorobenzene, tetrahydrofuran, propan-2-one, butan-2-one, pentan-2-one, 4-methylpentan-2-one, dichlorobenzene, dimethylformamide, dimethylacetamide, N-methyl-2-pyrrolidone, dimethyl sulfoxide, N,N′-dimethylpropyleneurea, sulfolane, and a combination thereof.

53. The process according to any one of statements 1 to 52, wherein the desulfinylation step is carried out in the presence of at least one diluent, preferably at least one solvent, preferably wherein the at least one solvent is selected from the group comprising toluene, o-xylene, 1,2-dichloroethane, chloroform, 2-methyltetrahydrofuran, ethyl acetate, anisole, chlorobenzene, tetrahydrofuran, acetone, dichlorobenzene, and a combination thereof.

54. The process according to any one of statements 1 to 53, wherein compound of formula (II) is a compound of formula (II′) and compound of formula (I) is a compound of formula (I′)

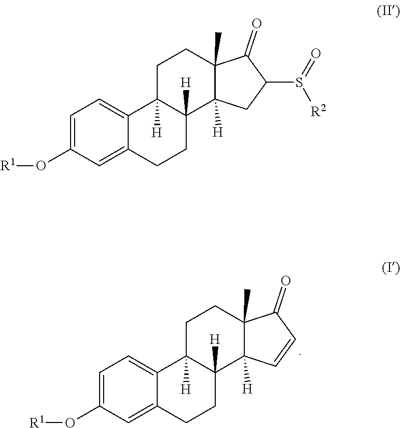

55. The process according to any one of statements 1 to 54, wherein compound of formula (II) is obtained by a process comprising the steps of:
a) protecting the hydroxyl of a compound of formula (III) to produce a compound of formula (IV), wherein R¹ has the same meaning as in statement 1 to 3;

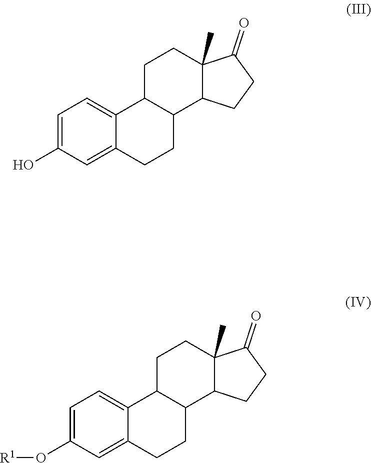

b) sulfinylation of compound of formula (IV) to produce compound of formula (II).

56. The process according to statement 55, wherein the sulfinylation is performed by reaction of compound of formula (IV) with at least one sulfinylation reagent, preferably in the presence of at least one suitable base.

57. The process according to any one of statements 55 to 56, wherein the sulfinylation is performed by reacting compound of formula (IV) with at least one sulfinylation reagent, preferably the at least one sulfinylation reagent is selected from the group comprising methyl benzene sulfinate, methyl 2-pyridinesulfinate, methyl 4-methyl-benzenesufinate, methyl 4-chloro-benzene sulfinate, methyl 4-bromo-benzene sulfinate, methyl 4-fluoro-benzene sulfinate, methyl 4-methoxy-benzene sulfinate, methyl 4-nitro-benzene sulfinate, methyl 2,3,4,5,6-pentafluorobenzenesulfinate, methyl 2,3,4,5,6-pentachlorobenzenesulfinate, methyl-4-(trifluoromethylsulfonyl)-benzenesulfinate, 1-(4-methylsulfnylphenyl) ethanone, methyl-4-cyano-benzenesulfinate, methyl-4-(dimethylamino)-benzenesulfinate, methyl 4-(methylsulfinate)-benzoate, and methyl 4-(2-benzothiazolyl)-benzenesulfinate.

58. The process according to any one of statements 55 to 57, wherein the sulfinylation reaction is performed in the presence of at least one suitable base, wherein the at least one base is selected from sodium hydride, sodium tert-butylate, potassium hydride, potassium tert-butylate, or a mixture thereof.

59. A composition obtained by the process according to any one of statements 1 to 58, wherein said composition comprises a compound of formula (I) and a compound of formula (IIa);

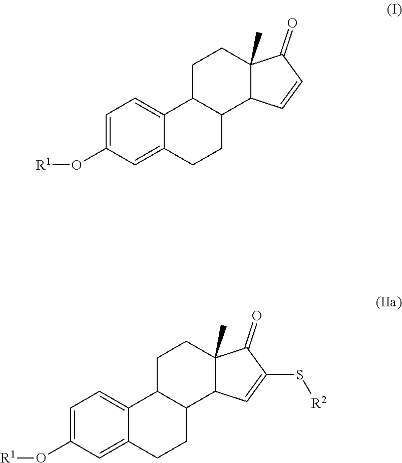

R¹ is selected from the group comprising hydrogen, R³Si(R⁴)(R⁵)—, C₁₋₆alkyl, C₆₋₁₀aryl, C₆₋₁₀arylC₁₋₆alkyl, —CH₂—CH═CR^{a}R^{b}, C₁₋₆alkoxy, C₃₋₆cycloalkyl, and R⁶CO—; each of said C₁₋₆alkyl, C₆₋₁₀aryl, C₆₋₁₀arylC₁₋₆alkyl, and C₃₋₆cycloalkyl may be optionally substituted with one or more substituents each independently selected from the group consisting of halo, C₁₋₆alkyl, haloC₁₋₆alkyl, C₁₋₆alkoxy, haloC₁₋₆alkoxy, C₆₋₁₀aryl, heterocyclyl, and nitro;
R² is C₆₋₁₀aryl, or heteroaryl; wherein each C₆₋₁₀aryl or heteroaryl may be optionally substituted with one or more substituents each independently selected from the group consisting of halo, C₁₋₆alkyl, haloC₁₋₆alkyl, C₁₋₆alkoxy, haloC₁₋₆alkoxy, C₂₋₆alkenyl, C₂₋₆alkynyl, C₆₋₁₀aryl, heterocyclyl, heteroaryl, hydroxyl, —S(O)₂R⁷, —S(O)R⁸, CO₂R⁹, C(O)R¹⁰, —SR¹³, —C(O)SR¹⁴, NR¹¹R¹², cyano and nitro;
R³, R⁴ and R⁵ are each independently C₁₋₆alkyl or C₆₋₁₀aryl, wherein each C₁₋₆alkyl or C₆₋₁₀aryl may be optionally substituted with one or more substituents each independently selected from the group consisting of halo, C₁₋₆alkyl, haloC₁₋₆alkyl, C₁₋₆alkoxy, and haloC₁₋₆alkoxy; preferably fluoro, chloro or C₁₋₄alkyl;
R⁶ is C₁₋₆alkyl, C₆₋₁₀aryl, or C₃₋₆cycloalkyl, wherein each C₁₋₆alkyl, C₆₋₁₀aryl, or C₃₋₆cycloalkyl may be optionally substituted by one or more substituents independently selected from the group consisting of halo, C₁₋₆alkyl, haloC₁₋₆alkyl, C₁₋₆alkoxy, and haloC₁₋₆alkoxy;
R⁷, R⁸, R⁹ and R¹⁰ are each independently selected from the group consisting of hydrogen, hydroxyl, C₁₆alkyl, C₆₁₀aryl, and C₃₋₆cycloalkyl, wherein each C₁₋₆alkyl, C₆₋₁₀aryl, or C₃₋₆cycloalkyl may be optionally substituted by one or more substituents independently selected from the group consisting of halo, C₁₋₆alkyl, haloC₁₋₆alkyl, C₁₋₆alkoxy, and haloC₁₋₆alkoxy;
R¹¹ and R¹² are each independently selected from the group consisting of hydrogen, C₁₋₆alkyl, C₆₋₁₀aryl, C₃₋₆cycloalkyl; wherein each C₁₋₆alkyl, C₆₋₁₀aryl, or C₃₋₆cycloalkyl may be optionally substituted by one or more substituents independently selected from the group consisting of halo, C₁₋₆alkyl, haloC₁₋₆alkyl, C₁₋₆alkoxy, and haloC₁₋₆alkoxy;
R¹³ and R¹⁴ are each independently selected from the group consisting of hydrogen, C₁₋₆alkyl, C₆₋₁₀aryl, and C₃₋₆cycloalkyl, wherein each C₁₋₆alkyl, C₆₋₁₀aryl, or C₃₋₆cycloalkyl may be optionally substituted by one or more substituents independently selected from the group consisting of halo, C₁₋₆alkyl, haloC₁₋₆alkyl, C₁₋₆alkoxy, and haloC₁₋₆alkoxy;
R^{a} and R^{b} are each independently hydrogen, C₁₋₆alkyl or C₆₋₁₀aryl.

60. A composition obtained by the process according to any one of statements 1 to 58, wherein said composition comprises a compound of formula (I) and a compound of formula (IIa);

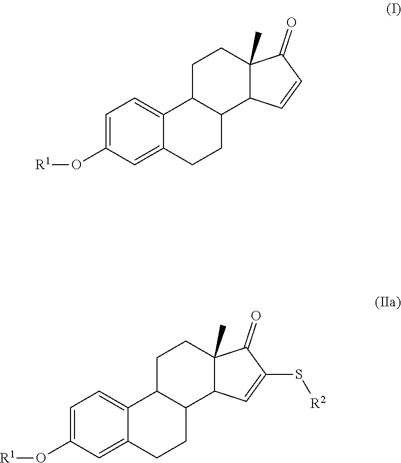

R¹ is selected from the group comprising hydrogen, R³Si(R⁴)(R⁵)—, C₁₋₆alkyl, C₆₋₁₀aryl, C₆₋₁₀arylC₁₋₆alkyl, —CH₂—CH═CR^{a}R^{b}, and R⁶CO—; each of said C₁₋₆alkyl, C₆₋₁₀aryl, and C₆₋₁₀arylC₁₋₆alkyl may be optionally substituted with one or more substituents each independently selected from the group consisting of halo, C₁₋₆alkyl, haloC₁₋₆alkyl, C₁₋₆alkoxy, haloC₁₋₆alkoxy, C₆₋₁₀aryl, heterocyclyl, and nitro;
R² is C₆₋₁₀aryl, or heteroaryl; wherein each C₆₋₁₀aryl or heteroaryl may be optionally substituted with one or more substituents each independently selected from the group consisting of halo, C₁₋₆alkyl, haloC₁₋₆alkyl, C₁₋₆alkoxy, haloC₁₋₆alkoxy, and nitro;
R³, R⁴ and R⁵ are each independently C₁₋₆alkyl or C₆₋₁₀aryl, wherein each C₁₋₆alkyl or C₆₋₁₀aryl may be optionally substituted with one or more substituents each independently selected from the group consisting of halo, C₁₋₆alkyl, haloC₁₋₆alkyl, C₁₋₆alkoxy, and haloC₁₋₆alkoxy; preferably fluoro, chloro or C₁₋₄alkyl;
R⁶ is C₁₋₆alkyl, C₆₋₁₀aryl, or C₃₋₆cycloalkyl, wherein each C₁₋₆alkyl, C₆₋₁₀aryl, or C₃₋₆cycloalkyl may be optionally substituted by one or more substituents independently selected from the group consisting of halo, C₁₋₆alkyl, haloC₁₋₆alkyl, C₁₋₆alkoxy, and haloC₁₋₆alkoxy;
R^{a} and R^{b} are each independently hydrogen, C₁₋₆alkyl or C₆₋₁₀aryl.

61. A composition, wherein said composition comprises a compound of formula (I) and a compound of formula (IIa);

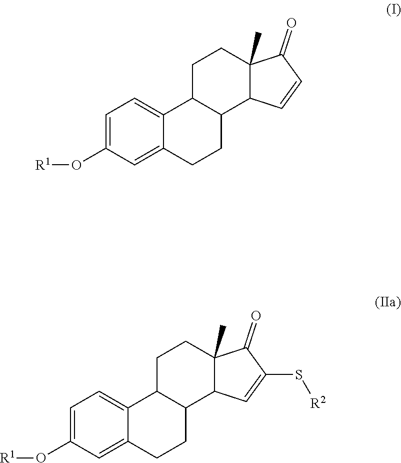

R¹ is selected from the group comprising hydrogen, R³Si(R⁴)(R⁵)—, C₁₋₆alkyl, C₆₋₁₀aryl, C₆₋₁₀arylC₁₋₆alkyl, —CH₂—CH═CR^{a}R^{b}, C₁₋₆alkoxy, C₃₋₆cycloalkyl, and R⁶CO—; each of said C₁₋₆alkyl, C₆₋₁₀aryl, C₆₋₁₀arylC₁₋₆alkyl, and C₃₋₆cycloalkyl may be optionally substituted with one or more substituents each independently selected from the group consisting of halo, C₁₋₆alkyl, haloC₁₋₆alkyl, C₁₋₆alkoxy, haloC₁₋₆alkoxy, C₆₋₁₀aryl, heterocyclyl, and nitro;
R² is C₆₋₁₀aryl, or heteroaryl; wherein each C₆₋₁₀aryl or heteroaryl may be optionally substituted with one or more substituents each independently selected from the group consisting of halo, C₁₋₆alkyl, haloC₁₋₆alkyl, C₁₋₆alkoxy, haloC₁₋₆alkoxy, C₂₋₆alkenyl, C₂₋₆alkynyl, C₆₋₁₀aryl, heterocyclyl, heteroaryl, hydroxyl, —S(O)₂R⁷, —S(O)R⁸, —CO₂R⁹, —C(O)R¹⁰, —SR¹³, —C(O)SR¹⁴, NR¹¹R¹², cyano and nitro;
R³, R⁴ and R⁵ are each independently C₁₋₆alkyl or C₆₋₁₀aryl, wherein each C₁₋₆alkyl or C₆₋₁₀aryl may be optionally substituted with one or more substituents each independently selected from the group consisting of halo, C₁₋₆alkyl, haloC₁₋₆alkyl, C₁₋₆alkoxy, and haloC₁₋₆alkoxy; preferably fluoro, chloro or C₁₋₄alkyl;
R⁶ is C₁₋₆alkyl, C₆₋₁₀aryl, or C₃₋₆cycloalkyl, wherein each C₁₋₆alkyl, C₆₋₁₀aryl, or C₃₋₆cycloalkyl may be optionally substituted by one or more substituents independently selected from the group consisting of halo, C₁₋₆alkyl, haloC₁₋₆alkyl, C₁₋₆alkoxy, and haloC₁₋₆alkoxy;
R⁷, R⁸, R⁹ and R¹⁰ are each independently selected from the group consisting of hydrogen, hydroxyl, C₁₆alkyl, C₆₁₀aryl, and C₃₋₆cycloalkyl, wherein each C₁₋₆alkyl, C₆₋₁₀aryl, or C₃₋₆cycloalkyl may be optionally substituted by one or more substituents independently selected from the group consisting of halo, C₁₋₆alkyl, haloC₁₋₆alkyl, C₁₋₆alkoxy, and haloC₁₋₆alkoxy;
R¹¹ and R¹² are each independently selected from the group consisting of hydrogen, C₁₋₆alkyl, C₆₋₁₂aryl, C₃₋₆cycloalkyl, wherein each C₁₋₆alkyl, C₆₋₁₀aryl, or C₃₋₆cycloalkyl may be optionally substituted by one or more substituents independently selected from the group consisting of halo, C₁₋₆alkyl, haloC₁₋₆alkyl, C₁₋₆alkoxy, and haloC₁₋₆alkoxy;
R¹³ and R¹⁴ are each independently selected from the group consisting of hydrogen, C₁₋₆alkyl, C₆₋₁₀aryl, and C₃₋₆cycloalkyl, wherein each C₁₋₆alkyl, C₆₋₁₀aryl, or C₃₋₆cycloalkyl may be optionally substituted by one or more substituents independently selected from the group consisting of halo, C₁₋₆alkyl, haloC₁₋₆alkyl, C₁₋₆alkoxy, and haloC₁₋₆alkoxy;
R^{a} and R^{b} are each independently hydrogen, C₁₋₆alkyl or C₆₋₁₀aryl.

62. The composition according to statements 59 to 61, wherein the composition also comprises a compound of formula (IIb);

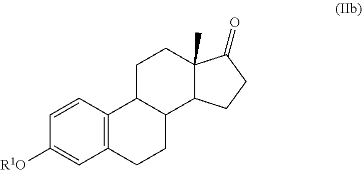

wherein R¹ is as defined in any one of statements 59 to 61.

63. The composition according to any one of statements 59 to 62, wherein the composition also comprises a compound of formula (IIc);

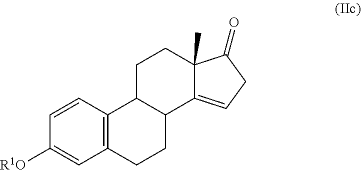

wherein R¹ is as defined in any one of statements 59 to 61.

64. The composition according to any one of statements 59 to 63, wherein the composition also comprises a compound of formula (IId);

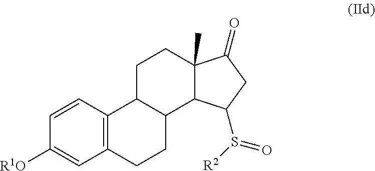

wherein R¹ and R² are as defined in any one of statements 59 to 61.

65. The composition according to any one of statements 59 to 64, wherein the composition comprises an amount of at least 0.01% area by HPLC of compound (IIa), based on the total area of the composition as determined by HPLC; preferably an amount of at least 0.1% area by HPLC of compound (IIa); preferably an amount of 0.5% area by HPLC of compound (IIa); preferably an amount of at least 1.0% area by HPLC of compound (IIa); wherein the HPLC method is as described in the Example section of the application.

66. The composition according to any one of statements 59 to 65, wherein the composition comprises an amount of at most 25.0% area by HPLC of compound (IIa), based on the total area of the composition as determined by HPLC; preferably an amount of at most 20.0% of compound (IIa); preferably an amount of at most 15.0% area by HPLC of compound (IIa); preferably an amount of at most 10.0% area by HPLC of compound (IIa); preferably an amount of at most 10.5% area by HPLC of compound (IIa); preferably an amount of at most 5.0% area by HPLC of compound (IIa); wherein the HPLC method is as described in the Example section of the application.

67. The composition according to any one of statements 59 to 66, wherein the composition comprises an amount of at most 16.0% area by HPLC of compound (IIb), based on the total area of the composition as determined by HPLC; preferably an amount of at most 10.0% area by HPLC of compound (IIb); preferably an amount of at most 5.0% area by HPLC of compound (IIb); wherein the HPLC method is as described in the Example section of the application.

68. The composition according to any one of statements 59 to 67, wherein the composition comprises an amount of at most 10.0% area by HPLC of compound (IIc), based on the total area of the composition as determined by HPLC; preferably an amount of at most 8.0% area by HPLC of compound (IIc); preferably an amount of at most 5.0% area by HPLC of compound (IIc); preferably an amount of at most 2.0% area by HPLC of compound (IIc); wherein the HPLC method is as described in the Example section of the application.

69. The composition according to any one of statements 59 to 68, wherein the composition comprises an amount of at most 3.0% area by HPLC of compound (IId), based on the total area of the composition as determined by HPLC; preferably an amount of at most 2.0% area by HPLC of compound (IId); preferably an amount of at most 1.0% area by HPLC of compound (IId); preferably an amount of at most 0.5% area by HPLC of compound (IId); wherein the HPLC method is as described in the Example section of the application.

70. Process for the preparation of estra-1,3,5(10),-triene 3,15α,16α,17β-tetrol of formula (E), a stereoisomer, a salt, a hydrate or a solvate thereof, said process comprising the step of preparing a compound of formula (I) by a process according to any of statements 1 to 58, and further reacting compound of formula (I) to produce estra-1,3,5(10),-triene 3,15α,16α,17β-tetrol of formula (E), a stereoisomer, a salt, a hydrate, an ester, an ether, or a solvate thereof,

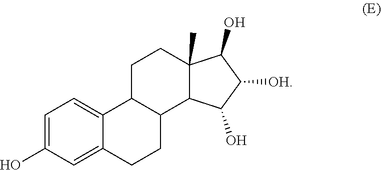 [SMILES: C[C@]12CCC3c4ccc(O)cc4CCC3C1[C@@H](O)[C@@H](O)[C@@H]2O]

71. Process according to statement 70, wherein estra-1,3,5(10),-triene 3,15α,16α,17β-tetrol of formula (E) is estetrol of formula (E′),

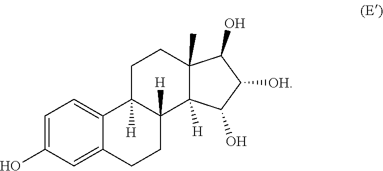

72. A process for the preparation of estra-1,3,5(10),-triene 3,15α,16α,17β-tetrol of formula (E), a stereoisomer, a salt, a hydrate, an ester, an ether, or a solvate thereof;

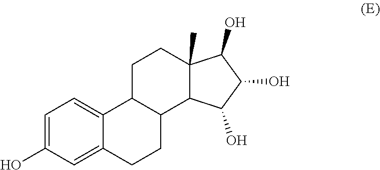 [SMILES: C[C@]12CCC3c4ccc(O)cc4CCC3C1[C@@H](O)[C@@H](O)[C@@H]2O]

said process comprising the steps of
a1) preparing a compound of formula (I) using a process according to any of statements 1 to 58;

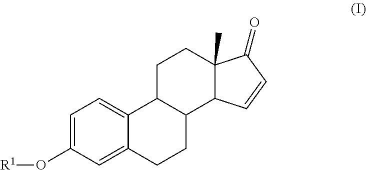

wherein
R¹ is selected from the group comprising hydrogen, R³Si(R⁴)(R⁵)—, C₁₋₆alkyl, C₆₋₁₀aryl, C₆₋₁₀arylC₁₋₆alkyl, —CH₂—CH═CR^{a}R^{b}, C₁₋₆alkoxy, C₃₋₆cycloalkyl, and R⁶CO—; each of said C₁₋₆alkyl, C₆₋₁₀aryl, C₆₋₁₀arylC₁₋₆alkyl, and C₃₋₆cycloalkyl may be optionally substituted with one or more substituents each independently selected from the group consisting of halo, C₁₋₆alkyl, haloC₁₋₆alkyl, C₁₋₆alkoxy, haloC₁₋₆alkoxy, C₆₋₁₀aryl, heterocyclyl, and nitro;
R³, R⁴ and R⁵ are each independently C₁₋₆alkyl or C₆₋₁₀aryl, wherein each C₁₋₆alkyl or C₆₋₁₀aryl may be optionally substituted with one or more substituents each independently selected from the group consisting of halo, C₁₋₆alkyl, haloC₁₋₆alkyl, C₁₋₆alkoxy, and haloC₁₋₆alkoxy; preferably fluoro, chloro or C₁₋₄alkyl;
R⁶ is C₁₋₆alkyl, C₆₋₁₀aryl, or C₃₋₆cycloalkyl, wherein each C₁₋₆alkyl, C₆₋₁₀aryl, or C₃₋₆cycloalkyl may be optionally substituted by one or more substituents independently selected from the group consisting of halo, C₁₋₆alkyl, haloC₁₋₆alkyl, C₁₋₆alkoxy, and haloC₁₋₆alkoxy;
R^{a} and R^{b} are each independently hydrogen, C₁₋₆alkyl or C₆₋₁₀aryl;
b1) contacting compound of formula (I) with a reducing agent to produce a compound of formula (A);

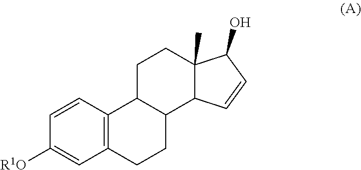

c1) contacting compound of formula (A), with an acylating or a silylating agent to produce a compound of formula (B),

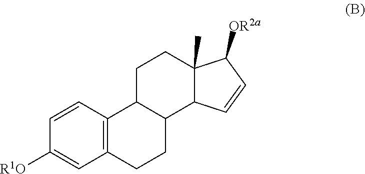

wherein
R^{2a} is a protecting group selected from (R⁷R⁸R⁹)C—CO—, or (R¹⁰)Si(R¹¹)(R¹²)—;
R⁷, R⁸ and R⁹ are each independently hydrogen, C₁₋₆alkyl or C₆₋₁₀aryl, wherein each of said C₁₋₆alkyl or C₆₋₁₀aryl may be optionally substituted with one or more substituents each independently selected from the group consisting of halo, C₁₋₆alkyl, haloC₁₋₆alkyl, C₁₋₆alkoxy, and haloC₁₋₆alkoxy;
R¹⁰, R¹¹ and R¹² are each independently C₁₋₆alkyl or C₆₋₁₀aryl, wherein each C₁₋₆alkyl or C₆₋₁₀aryl may be optionally substituted with one or more substituents each independently selected from the group consisting of halo, C₁₋₆alkyl, haloC₁₋₆alkyl, C₁₋₆alkoxy, and halo_{C1-6}alkoxy; preferably fluoro, chloro or C₁₋₄alkyl;
d1) contacting compound of formula (B) with at least one oxidizing agent to produce compound of formula (C); and

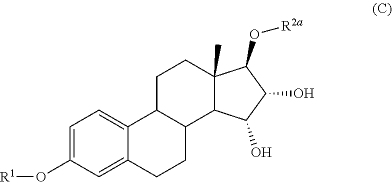

e1) deprotecting the compound of formula (C) to produce compound of formula (E).

73. The process according to statement 72, wherein R¹ is R³Si(R⁴)(R⁵)—, and R^{2a} is R¹⁰Si(R¹¹)(R¹²)—.

74. A process for the preparation of estra-1,3,5(10),-triene 3,15α,16α,17β-tetrol of formula (E), a stereoisomer, a salt, a hydrate, an ester, an ether, or a solvate thereof;

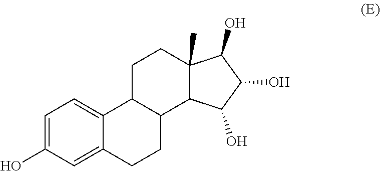 [SMILES: C[C@]12CCC3c4ccc(O)cc4CCC3C1[C@@H](O)[C@@H](O)[C@@H]2O]

said process comprising the steps of
a2) preparing a compound of formula (I) using a process according to any of statements 1 to 58;

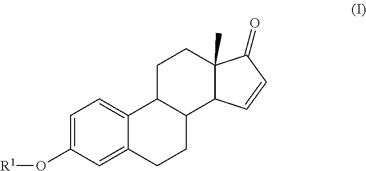

wherein
R¹ is benzyl;
b2) contacting compound of formula (I) with a reducing agent to produce a compound of formula (A),

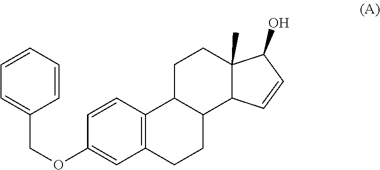 [SMILES: C[C@]12CCC3c4ccc(OCc5ccccc5)cc4CCC3C1C=C[C@@H]2O]

c2) contacting compound of formula (A) with at least one oxidizing agent to produce compound of formula (K),

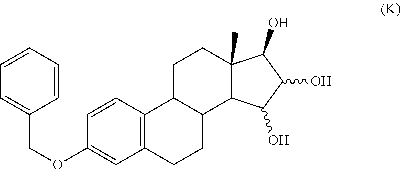 [SMILES: C[C@]12CCC3c4ccc(OCc5ccccc5)cc4CCC3C1C(O)C(O)[C@@H]2O]

d2) contacting compound of formula (K), with an acylating agent to produce a compound of formula (L),

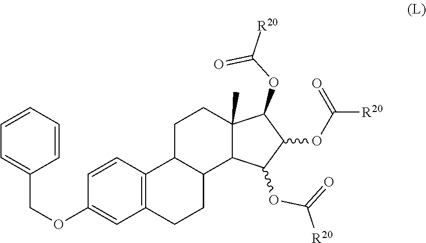

wherein
R²⁰ is selected from hydrogen or methyl;
e2) removing the benzyl protecting group in position 3 by transfer or catalytic hydrogenation to give a compound of general formula (M),

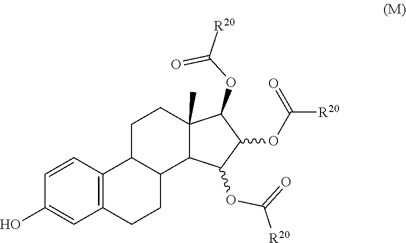

f2) deprotecting the compound of formula (M) with an alkali metal carbonate, alkali metal hydrogen carbonate or alkali metal hydroxide in a suitable solvent to produce compound (E).

75. The process according to statement 74, wherein the acylating agent in step d2) is selected from the group consisting of acetic anhydride, acetyl chloride, acetic acid-formic acid mixed anhydride and acetyl bromide.

76. The process according to any one of statements 74 to 75 wherein step d2) is carried out in the presence of a tertiary amine base.

77. A process for the preparation of estra-1,3,5(10),-triene 3,15α,16α,17β-tetrol of formula (E), a stereoisomer, a salt, a hydrate or a solvate thereof;

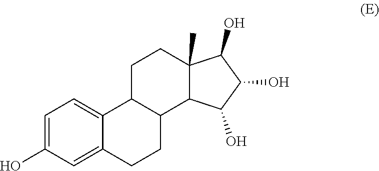 [SMILES: C[C@]12CCC3c4ccc(O)cc4CCC3C1[C@@H](O)[C@@H](O)[C@@H]2O]

said process comprising the steps of
a3) preparing a compound of formula (I) using a process according to any of statements 1 to 58;

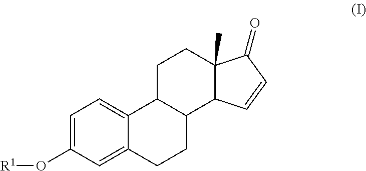

wherein
R¹ is selected from the group comprising hydrogen, R³Si(R⁴)(R⁵)—, C₁₋₆alkyl, C₆₋₁₀aryl, C₆₋₁₀arylC₁₋₆alkyl, —CH₂—CH═CR^{a}R^{b}, C₁₋₆alkoxy, C₃₋₆cycloalkyl, and R⁶CO—; each of said C₁₋₆alkyl, C₆₋₁₀aryl, C₆₋₁₀arylC₁₋₆alkyl, and C₃₋₆cycloalkyl may be optionally substituted with one or more substituents each independently selected from the group consisting of halo, C₁₋₆alkyl, haloC₁₋₆alkyl, C₁₋₆alkoxy, haloC₁₋₆alkoxy, C₆₋₁₀aryl, heterocyclyl, and nitro;
R³, R⁴ and R⁵ are each independently C₁₋₆alkyl or C₆₋₁₀aryl, wherein each C₁₋₆alkyl or C₆₋₁₀aryl may be optionally substituted with one or more substituents each independently selected from the group consisting of halo, C₁₋₆alkyl, haloC₁₋₆alkyl, C₁₋₆alkoxy, and haloC₁₋₆alkoxy; preferably fluoro, chloro or C₁₋₄alkyl;
R⁶ is C₁₋₆alkyl, C₆₋₁₀aryl, or C₃₋₆cycloalkyl, wherein each C₁₋₆alkyl, C₆₋₁₀aryl, or C₃₋₆cycloalkyl may be optionally substituted by one or more substituents independently selected from the group consisting of halo, C₁₆alkyl, haloC₁₋₆alkyl, C₁₋₆alkoxy, and haloC₁₋₆alkoxy;
R^{a} and R^{b} are each independently hydrogen, C₁₋₆alkyl or C₆₋₁₀aryl;
b3) contacting compound of formula (I) with a reducing agent to produce a compound of formula (A),

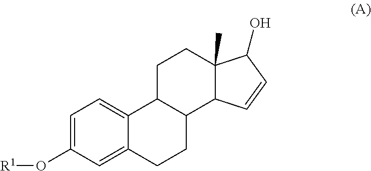

c3) removing protecting group R¹ from compound of formula (A) to produce a diol;
d3) protecting the hydroxyl group at C3 of the diol obtained in step c) to produce a compound of formula (G),

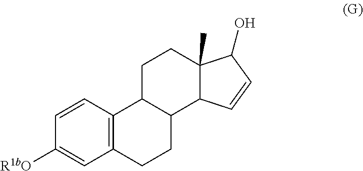

wherein,
R^{1b} is a protecting group selected from R¹³—CO—, R¹⁴Si(R¹⁵)(R¹⁶)—, C₁₋₆alkyl, C₆₋₁₀aryl, C₆₋₁₀arylC₁₋₆alkyl, C₃₋₆cycloalkyl, heterocyclyl, heteroaryl, R¹⁷R¹⁸N—CO—, or R¹⁹—OCO—; preferably R¹³—CO—, C₁₋₆alkyl, C₆₋₁₀aryl, C₆₋₁₀arylthioC₁₋₆alkyl, heterocyclyl, or heteroaryl; preferably R¹³—CO—, C₁₋₄alkyl, C₆₋₁₀aryl, C₆₋₁₀arylthioC₁₋₆alkyl, heterocyclyl, or heteroaryl; wherein each C₁₋₆alkyl, C₆₋₁₀aryl, C₆₋₁₀arylC₁₋₆alkyl, C₃₋₆cycloalkyl, heterocyclyl or heteroaryl, may be optionally substituted with one or more substituents each independently selected from the group consisting of halo, C₁₋₆alkyl, haloC₁₋₆alkyl, C₁₋₆alkoxy, C₁₋₆alkylthio and haloC₁₋₆alkoxy; preferably fluoro, chloro or C₁₋₄alkyl; preferably R^{1b} is selected from the group comprising R¹³—CO—, 1-butoxyethyl, tetrahydropyranyl, phenylthiomethyl, and methyloxymethyl;
R¹³ is selected from the group consisting of C₁₋₆alkyl, C₆₋₁₀aryl, C₆₋₁₀arylC₁₋₆alkyl, C₃₋₆cycloalkyl, heterocyclyl or heteroaryl, wherein each C₁₋₆alkyl C₆₋₁₀aryl, C₆₋₁₀arylC₁₋₆alkyl, C₃₋₆cycloalkyl, heterocyclyl or heteroaryl, may be optionally substituted with one or more substituents each independently selected from the group consisting of halo, C₁₋₆alkyl, haloC₁₋₆alkyl, C₁₋₆alkoxy, C₁₋₆alkylthio and haloC₁₋₆alkoxy; preferably fluoro, chloro or C₁₋₄alkyl;
R¹⁴, R¹⁵ and R¹⁶ are each independently C₁₋₆alkyl, C₃₋₆cycloalkyl, or C₆₋₁₀aryl, wherein each C₁₋₆alkyl, C₃₋₆cycloalkyl, or C₆₋₁₀aryl may be optionally substituted with one or more substituents each independently selected from the group consisting of halo, C₁₋₆alkyl, haloC₁₋₆alkyl, C₁₋₆alkoxy, and haloC₁₋₆alkoxy; preferably fluoro, chloro or C₁₋₄alkyl;
R¹⁷ and R¹⁸ are each independently selected from the group consisting of C₁₋₆alkyl, C₆₋₁₀aryl, C₆₋₁₀arylC₁₋₆alkyl, and C₃₋₆cycloalkyl, wherein each C₁₋₆alkyl, C₆₋₁₀aryl, C₆₋₁₀arylC₁₋₆alkyl, or C₃₋₆cycloalkyl may be optionally substituted with one or more substituents each independently selected from the group consisting of halo, C₁₋₆alkyl, haloC₁₋₆alkyl, C₁₋₆alkoxy, and haloC₁₋₆alkoxy; preferably fluoro, chloro or C₁₋₄alkyl;
R¹⁹ is selected from the group consisting of C₁₋₆alkyl, C₆₋₁₀aryl, C₆₋₁₀arylC₁₋₆alkyl, C₃₋₆cycloalkyl, heterocyclyl, and heteroaryl, wherein each C₁₋₆alkyl C₆₋₁₀aryl, C₆₋₁₀arylC₁₋₆alkyl, C₃₋₆cycloalkyl, heterocyclyl or heteroaryl, may be optionally substituted with one or more substituents each independently selected from the group consisting of halo, C₁₋₆alkyl, haloC₁₋₆alkyl, C₁₋₆alkoxy, and haloC₁₋₆alkoxy; preferably fluoro, chloro or C₁₋₄alkyl;
e3) optionally protecting the hydroxyl group at C17 of the compound of formula (G) to produce a compound of formula (H),

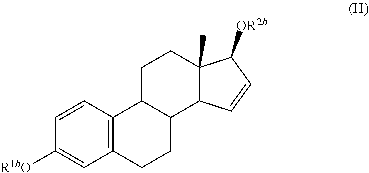

wherein
R^{2b} is a protecting group selected from the group consisting of C₁₋₆alkyl, C₆₋₁₀aryl, C₆₋₁₀arylthioC₁₋₆alkyl, heterocyclyl, and heteroaryl; preferably C₁₋₄alkyl, C₆₋₁₀aryl, heterocyclyl, or heteroaryl; wherein each of said C₁₋₆alkyl, C₆₋₁₀aryl, heterocyclyl or heteroaryl, may be optionally substituted with one or more substituents each independently selected from the group consisting of halo, C₁₋₆alkyl, haloC₁₋₆alkyl, C₁₋₆alkoxy, C₁₋₆alkylthio and haloC₁₋₆alkoxy; preferably fluoro, chloro, C₁₋₄alkoxy or C₁₋₄alkyl; preferably R^{2b} is selected from the group consisting of 1-butoxyethyl, tetrahydropyranyl, phenylthiomethyl and methyloxymethyl;
f3) contacting compound of formula (G) or the compound of formula (H) with at least one oxidizing agent selected from the group comprising permanganate salt, osmium oxide, potassium osmate, hydrogen peroxide, iodine, and silver acetate to produce a compound of formula (E) or a compound of formula (J); and

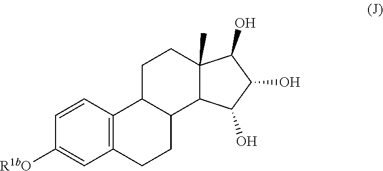

g3) if a compound of formula (J) is obtained in step f3), deprotecting the compound of formula (J) to produce compound of formula (E).

78. The process according to any one of statements 72 to 77, wherein said at least one oxidizing agent is selected from the group comprising permanganate salt, osmium oxide, potassium osmate, hydrogen peroxide, iodine, and silver acetate.

79. The process according to any one of statements 72 to 78, wherein said at least one oxidizing agent is supported osmium oxide.

80. The process according to any one of statements 72 to 79, wherein said at least one oxidizing agent is osmium oxide immobilized on poly(4-vinyl-pyridine) (PVP).

81. The process according to any one of statements 72 to 80, wherein said at least one oxidizing agent is used in combination with a co-oxidant.

82. The process according to any one of statements 72 to 81, wherein said at least one oxidizing agent is used in combination with a co-oxidant selected from the group consisting of trialkylamine-N-oxide, N-methyl morpholine-N-oxide and hydrogen peroxide; preferably trimethylamine N-oxide, triethylamine N-oxide, N-methyl morpholine-N-oxide or hydrogen peroxide.

83. The process according to any one of statements 72 to 82, wherein said at least one oxidizing agent is osmium oxide immobilized on PVP in combination with a co-oxidant.

84. The process according to any one of statements 72 to 83, wherein said at least one oxidizing agent is osmium oxide immobilized on PVP in combination with a co-oxidant selected from the group consisting of trialkylamine-N-oxide, N-methyl morpholine-N-oxide and hydrogen peroxide; preferably trimethylamine N-oxide, triethylamine N-oxide, N-methyl morpholine-N-oxide or hydrogen peroxide.

85. The process according to statement 72 to 84 wherein R^{1b} and R^{2b} are the same protecting group.

86. The process according to any one of statements 72 to 85, wherein compound of formula (E) is estetrol of formula (E′),

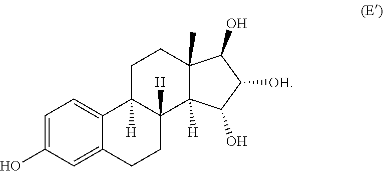

87. Production line for preparing a compound of formula (I) by continuous flow process comprising:
(a) sources of reactants including:
a source of compound of formula (II),
a source of diluent, and
optionally a source of at least one scavenger and optionally a source of at least one base, preferably at least one organic base;
(b) a diluent tank (preferably solution tank) in fluid communication with the sources of reactants,
(c) at least one pump bringing in fluid communication the diluent tank (preferably solution tank) with a reaction zone, wherein
(d) the reaction zone comprises:
a channel extending from an inlet to an outlet located downstream from the inlet, wherein at least one pump is located directly upstream of the inlet,
a heating module arranged for heating the channel at a reaction temperature,
optionally a cooling zone for cooling the mixture exiting the channel, said cooling zone being located downstream of the outlet, and
(e) optionally a separating zone for separating at least one compound of formula (I) from other reaction products.

88. The production line according to statement 87, wherein the diluent is a solvent.

89. The production line according to any one of statements 87 to 88, wherein the reaction zone is a thermolysis reaction zone.

90. Production line for preparing a compound of formula (I) by thermolysis comprising:
(a) sources of reactants including:
a source of compound of formula (II),
a source of solvent, and
optionally a source of at least one scavenger and optionally a source of at least one base, preferably at least one organic base;
(b) a solution tank in fluid communication with the sources of reactants,
(c) at least one pump bringing in fluid communication the solution tank with a thermolysis reaction zone, wherein
(d) the thermolysis reaction zone comprises:
a channel extending from an inlet to an outlet located downstream from the inlet, wherein at least one pump is located directly upstream of the inlet,
a heating module arranged for heating the channel at a thermolysis temperature,
optionally a cooling zone for cooling the solution exiting the channel, said cooling zone being located downstream of the outlet, and
(e) optionally a separating zone for separating at least one compound of formula (I) from other thermolysis products.

91. The process according to any one of statements 1 to 86, comprising after reaction contacting the reaction mixture comprising compound of formula (I) and other reaction products with an aqueous solution.

92. The process according to statement 91, wherein the aqueous solution comprises an inorganic base.

93. The process according to any one of statements 91 to 92, wherein the aqueous solution comprises an inorganic base selected from the group comprising NaOH, KOH, NaHCO₃, K₂CO₃, NH₄Cl, Na₂S₂O₃, and mixture thereof, preferably said aqueous solution comprises NaOH.

94. The process according to any one of statements 91 to 93, wherein the aqueous solution is a 0.5 mol/L to 2 mol/L NaOH solution, preferably a 1 mol/L NaOH solution.

95. The process according to any one of statements 91 to 94, wherein said reaction mixture is contacted with said aqueous solution and stirred for at least 15 minutes to at most 10 hours; preferably for at least 15 minutes to at most 5 hours; preferably for at least 30 minutes to at most 2 hours.

96. The process according to any one of statements 91 to 95, wherein said reaction mixture is a cooled reaction mixture.

Any reference to a “compound” also includes isomers such as stereoisomers and tautomers, salts such as pharmaceutically and/or physiologically acceptable salts, hydrates, ethers, esters and solvates of such compounds unless expressly indicated otherwise.

The term “isomers” as used herein means all possible isomeric forms, including tautomeric and stereochemical forms, which the compounds of formulae herein may possess, but not including position isomers. Typically, the structures shown herein exemplify one tautomeric or resonance form of the compounds, but the corresponding alternative configurations are contemplated as well.

The compounds of the invention may exist in solvated and not solvated forms. The term “solvates” refers to crystals formed by an active compound and a second component (solvent) which, in isolated form, is liquid at room temperature. Such solvates may be formed with common organic solvents, e.g., hydrocarbon solvents such as benzene or toluene; chlorinated solvents such as chloroform or dichloromethane; alcoholic solvents such as methanol, ethanol, or isopropanol; ethereal solvents such as diethyl ether or tetrahydrofuran; or ester solvents such as ethyl acetate. Alternatively, the solvates of the compounds herein may be formed with water, in which case they will be hydrates.

A currently accepted classification system for organic hydrates is one that defines isolated site, channel, or metal-ion coordinated hydrates—see Polymorphism in Pharmaceutical Solids by K. R. Morris (Ed. H. G. Britain, Marcel Dekker, 1995), incorporated herein by reference. Isolated site hydrates are ones in which the water molecules are isolated from direct contact with each other by intervening organic molecules. In channel hydrates, the water molecules lie in lattice channels where they are next to other water molecules. In metal-ion coordinated hydrates, the water molecules are bonded to the metal ion.

When the solvent or water is tightly bound, the complex will have a well-defined stoichiometry independent of humidity. When, however, the solvent or water is weakly bound, as in channel solvates and hygroscopic compounds, the water/solvent content will be dependent on humidity and drying conditions. In such cases, non-stoichiometry will be the norm.

Also included within the scope of the invention are multi-component complexes (other than salts and solvates) wherein the drug and at least one other component are present in stoichiometric or non-stoichiometric amounts. Complexes of this type include clathrates (drug-host inclusion complexes) and co-crystals. The latter are typically defined as crystalline complexes of neutral molecular constituents which are bound together through non-covalent interactions, but could also be a complex of a neutral molecule with a salt. Co-crystals may be prepared by melt crystallization, by recrystallization from solvents, or by physically grinding the components together—see Chem. Commun., 17, 1889-1896, by O. Almarsson and M. J. Zaworotko (2004), incorporated herein by reference. For a general review of multi-component complexes, see J. Pharm. Sci., 64 (8), 1269-1288, by Haleblian (August 1975), incorporated herein by reference.

The compounds of the invention may also exist in a mesomorphic state (mesophase or liquid crystal) when subjected to suitable conditions. The mesomorphic state is intermediate between the true crystalline state and the true liquid state (either melt or solution). Mesomorphism arising as the result of a change in temperature is described as ‘thermotropic’ and that resulting from the addition of a second component, such as water or another solvent, is described as ‘lyotropic’. Compounds that have the potential to form lyotropic mesophases are described as ‘amphiphilic’ and consist of molecules which possess an ionic (such as —COO⁻Na⁺, —COO⁻K⁺, or —SO₃⁻Na⁺) or non-ionic (such as —N⁻N⁺(CH₃)₃) polar head group. For more information, see Crystals and the Polarizing Microscope by N. H. Hartshorne and A. Stuart, 4^{th} Edition (Edward Arnold, 1970), incorporated herein by reference.

The present invention provides a continuous-flow process for the preparation of a compound of formula (I), a stereoisomer, a salt, a hydrate or a solvate thereof,

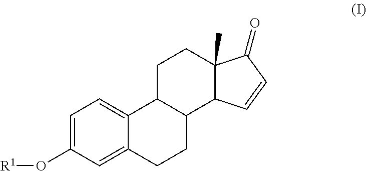

comprising the step of desulfinylation of a compound of formula (II) to produce compound of formula (I);

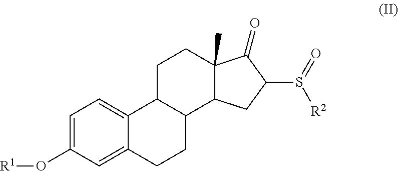

wherein:
R¹ is selected from the group comprising hydrogen, R³Si(R⁴)(R⁵)—, C₁₋₆alkyl, C₆₋₁₀aryl, C₆₋₁₀arylC₁₋₆alkyl, —CH₂—CH═CR^{a}R^{b}, C₁₋₆alkoxy, C₃₋₆cycloalkyl, and R⁶CO—; each of said C₁₋₆alkyl, C₆₋₁₀aryl, C₆₋₁₀arylC₁₋₆alkyl, C₃₋₆cycloalkyl, may be optionally substituted with one or more substituents each independently selected from the group consisting of halo, C₁₋₆alkyl, haloC₁₋₆alkyl, C₁₋₆alkoxy, haloC₁₋₆alkoxy, C₆₋₁₀aryl, heterocyclyl, and nitro; preferably R¹ is selected from the group comprising R³Si(R⁴)(R⁵)—, C₁₋₄alkyl, phenyl, C₆₋₁₀arylC₁₋₄alkyl, —CH₂—CH═CR^{a}R^{b}, C₁₋₄alkoxy, C₃₋₆cycloalkyl, and R⁶CO—; each of said C₁₋₄alkyl, phenyl, C₆₋₁₀arylC₁₋₄alkyl, or C₃₋₆cycloalkyl may be optionally substituted with one or more substituents each independently selected from the group consisting of halo, C₁₋₆alkyl, haloC₁₋₆alkyl, C₁₋₆alkoxy, haloC₁₋₆alkoxy, and nitro; preferably R¹ is selected from the group comprising R³Si(R⁴)(R⁵)—, C₁₋₄alkyl, phenyl, C₆₋₁₀arylC₁₋₄alkyl, —CH₂—CH═CR^{a}R^{b}, C₁₋₄alkoxy, C₃₋₆cycloalkyl, and R⁶CO—; each of said C₁₋₄alkyl, phenyl, C₆₋₁₀arylC₁₋₄alkyl, or C₃₋₆cycloalkyl may be optionally substituted with one or more substituents each independently selected from the group consisting of fluoro, chloro, bromo, C₁₋₄alkyl, haloC₁₋₄alkyl, C₁₋₄alkoxy, haloC₁₋₄alkoxy, and nitro; preferably R¹ is selected from the group comprising R³Si(R⁴)(R⁵)—, C₁₋₆alkyl, C₆₋₁₀aryl, C₆₋₁₀arylC₁₋₆alkyl, and R⁶CO—; each of said C₁₋₆alkyl, C₆₋₁₀aryl, and C₆₋₁₀arylC₁₋₆alkyl may be optionally substituted with one or more substituents each independently selected from the group consisting of halo, C₁₋₆alkyl, haloC₁₋₆alkyl, C₁₋₆alkoxy, haloC₁₋₆alkoxy, and nitro; preferably R¹ is selected from the group comprising R³Si(R⁴)(R⁵)—, C₁₋₆alkyl, C₆₋₁₀aryl, and C₆₋₁₀arylC₁₋₆alkyl; preferably R¹ is R³Si(R⁴)(R⁵)—; preferably R¹ is selected from the group comprising R³Si(R⁴)(R⁵)—, C₁₋₄alkyl, C₆₋₁₀aryl, and C₆₋₁₀arylC₁₋₄alkyl; preferably R¹ is R³Si(R⁴)(R⁵)—;
R² is C₆₋₁₀aryl, or heteroaryl; wherein each C₆₋₁₀aryl or heteroaryl may be optionally substituted with one or more substituents each independently selected from the group consisting of halo, C₁₋₆alkyl, haloC₁₋₆alkyl, C₁₋₆alkoxy, haloC₁₋₆alkoxy, C₂₋₆alkenyl, C₂₋₆alkynyl, C₆₋₁₀aryl, heterocyclyl, heteroaryl, hydroxyl, —S(O)₂R⁷, —S(O)R⁸, —CO₂R⁹, —C(O)R¹⁰, —SR¹³, —C(O)SR¹⁴, NR¹¹R¹², cyano and nitro; preferably fluoro, chloro, bromo, nitro, C₁₋₄alkyl, C₁₋₄alkyloxy, haloC₁₋₄alkyloxy C₆₋₁₀aryl, heteroaryl, —S(O)₂R⁷, CO₂R⁹, C(O)R¹⁰, NR¹¹R¹² or cyano; preferably, R² is C₆₋₁₀aryl, or 5-6 membered heteroaryl containing at least one N and/or S; wherein each C₆₋₁₀aryl or heteroaryl may be optionally substituted with one or more substituents each independently selected from the group consisting of halo, C₁₋₆alkyl, haloC₁₋₆alkyl, C₁₋₆alkoxy, haloC₁₋₆alkoxy, C₂₋₆alkenyl, C₂₋₆alkynyl, C₆₋₁₀aryl, heterocyclyl, heteroaryl, hydroxyl, —S(O)₂R⁷, —S(O)R⁸, —CO₂R⁹, —C(O)R¹⁰, NR¹¹R¹², cyano and nitro; preferably fluoro, chloro, bromo, nitro, C₁₋₄alkyl, C₁₋₄alkyloxy, haloC₁₋₄alkyloxy, C₂₋₆alkenyl, C₂₋₆alkynyl, C₆₋₁₀aryl, heteroaryl, —S(O)₂R⁷, —CO₂R⁹, —C(O)R¹⁰, NR¹¹R¹² or cyano; preferably, R² is C₆₋₁₀aryl, or 5-6 membered heteroaryl containing at least one N; wherein each C₆₋₁₀aryl or heteroaryl may be optionally substituted with one or more substituents each independently selected from the group consisting of halo, C₁₋₆alkyl, haloC₁₋₆alkyl, C₁₋₆alkoxy, haloC₁₋₆alkoxy, C₂₋₆alkenyl, C₂₋₆alkynyl, C₆₋₁₀aryl, heterocyclyl, heteroaryl, hydroxyl, —S(O)₂R⁷, —S(O)R⁸, —CO₂R⁹, —C(O)R¹⁰, —SR¹³, —C(O)SR¹⁴, NR¹¹R¹², cyano and nitro; preferably fluoro, chloro, bromo, nitro, C₁₋₄alkyl, C₁₋₄alkyloxy, haloC₁₋₄alkyloxy, C₂₋₆alkenyl, C₂₋₆alkynyl, C₆₋₁₀aryl, heteroaryl, —S(O)₂R⁷, —S(O)R⁸, CO₂R⁹, C(O)R¹⁰, NR¹¹R¹², or cyano;
R³, R⁴ and R⁵ are each independently C₁₋₆alkyl or C₆₋₁₀aryl, wherein each C₁₋₆alkyl or C₆₋₁₀aryl may be optionally substituted with one or more substituents each independently selected from the group consisting of halo, C₁₋₆alkyl, haloC₁₋₆alkyl, C₁₋₆alkoxy, and haloC₁₋₆alkoxy; preferably fluoro, chloro or C₁₋₄alkyl;
R⁶ is C₁₋₆alkyl or C₃₋₆cycloalkyl, wherein each C₁₋₆alkyl or C₃₋₆cycloalkyl may be optionally substituted by one or more substituents independently selected from the group consisting of halo, C₁₋₆alkyl, haloC₁₋₆alkyl, C₁₋₆alkoxy, and haloC₁₋₆alkoxy; preferably fluoro, chloro or C₁₋₄alkyl;
R⁷, R⁸, R⁹ and R¹⁰ are each independently selected from the group consisting of hydrogen, hydroxyl, C₁₆alkyl, C₆₁₀aryl, and C₃₋₆cycloalkyl, wherein each C₁₋₆alkyl, C₆₋₁₀aryl, or C₃₋₆cycloalkyl may be optionally substituted by one or more substituents independently selected from the group consisting of halo, C₁₋₆alkyl, haloC₁₋₆alkyl, C₁₋₆alkoxy, and haloC₁₋₆alkoxy; preferably R⁷, R⁸, R⁹ and R¹⁰ are each independently selected from the group consisting of hydrogen, hydroxyl, C₁₋₄alkyl, phenyl, and C₃₋₆cycloalkyl, wherein each C₁₋₄alkyl, phenyl, or C₃₋₆cycloalkyl may be optionally substituted by one or more substituents independently selected from the group consisting of halo, C₁₋₆alkyl, haloC₁₋₆alkyl, C₁₋₆alkoxy, and haloC₁₋₆alkoxy; preferably fluoro, chloro, bromo, iodo, C₁₋₄alkyl, haloC₁₋₄alkyl, C₁₋₄alkoxy, and haloC₁₋₄alkoxy; preferably R⁷, R⁸, R° and R¹⁰ are each independently selected from the group consisting of C₁₋₄alkyl and phenyl, wherein each C₁₋₄alkyl, or phenyl may be optionally substituted by one or more substituents independently selected from the group consisting of halo, C₁₋₆alkyl, haloC₁₋₆alkyl, C₁₋₆alkoxy, and haloC₁₋₆alkoxy; preferably fluoro, chloro, bromo, iodo, C₁₋₄alkyl, haloC₁₋₄alkyl, C₁₋₄alkoxy, and haloC₁₋₄alkoxy;
R¹¹ and R¹² are each independently selected from the group consisting of hydrogen, C₁₋₆alkyl, C₆₋₁₂aryl, C₃₋₆cycloalkyl; wherein each C₁₋₆alkyl, C₆₋₁₀aryl, or C₃₋₆cycloalkyl may be optionally substituted by one or more substituents independently selected from the group consisting of halo, C₁₋₆alkyl, haloC₁₋₆alkyl, C₁₋₆alkoxy, and haloC₁₋₆alkoxy; preferably R¹¹ and R¹² are each independently selected from the group consisting of hydrogen, C₁₋₄alkyl, phenyl, C₃₋₆cycloalkyl, wherein each C₁₋₄alkyl, phenyl, or C₃₋₆cycloalkyl may be optionally substituted by one or more substituents independently selected from the group consisting of halo, C₁₋₆alkyl, haloC₁₋₆alkyl, C₁₋₆alkoxy, and haloC₁₋₆alkoxy; preferably fluoro, chloro, bromo, iodo, C₁₋₄alkyl, haloC₁₋₄alkyl, C₁₋₄alkoxy, and haloC₁₋₄alkoxy; preferably R¹¹ and R¹² are each independently selected from the group consisting of C₁₋₄alkyl and phenyl, wherein each C₁₋₄alkyl, or phenyl may be optionally substituted by one or more substituents independently selected from the group consisting of halo, C₁₋₆alkyl, haloC₁₋₆alkyl, C₁₋₆alkoxy, and haloC₁₋₆alkoxy; preferably fluoro, chloro, bromo, iodo, C₁₋₄alkyl, haloC₁₋₄alkyl, C₁₋₄alkoxy, and haloC₁₋₄alkoxy;
R¹³ and R¹⁴ are each independently selected from the group consisting of hydrogen, C₁₋₆alkyl, C₆₋₁₀aryl, and C₃₋₆cycloalkyl, wherein each C₁₋₆alkyl, C₆₋₁₀aryl, or C₃₋₆cycloalkyl may be optionally substituted by one or more substituents independently selected from the group consisting of halo, C₁₋₆alkyl, haloC₁₋₆alkyl, C₁₋₆alkoxy, and haloC₁₋₆alkoxy; preferably R¹³ and
R¹⁴ are each independently selected from the group consisting of hydrogen, C₁₋₄alkyl, phenyl, C₃₋₆cycloalkyl, wherein each C₁₋₄alkyl, phenyl, or C₃₋₆cycloalkyl may be optionally substituted by one or more substituents independently selected from the group consisting of halo, C₁₋₆alkyl, haloC₁₋₆alkyl, C₁₋₆alkoxy, and haloC₁₋₆alkoxy; preferably fluoro, chloro, bromo, iodo, C₁₋₄alkyl, haloC₁₋₄alkyl, C₁₋₄alkoxy, and haloC₁₋₄alkoxy; preferably R¹³ and R¹⁴ are each independently selected from the group consisting of C₁₋₄alkyl and phenyl, wherein each C₁₋₄alkyl, or phenyl may be optionally substituted by one or more substituents independently selected from the group consisting of halo, C₁₋₆alkyl, haloC₁₋₆alkyl, C₁₋₆alkoxy, and haloC₁₋₆alkoxy; preferably fluoro, chloro, bromo, iodo, C₁₋₄alkyl, haloC₁₋₄alkyl, C₁₋₄alkoxy, and haloC₁₋₄alkoxy;
R^{a} and R^{b} are each independently hydrogen, C₁₋₆alkyl or C₆₋₁₀aryl;
preferably wherein said desulfinylation step is performed by thermolysis.

The present invention also provides a continuous-flow process for the preparation of a compound of formula (I), a stereoisomer, a salt, a hydrate or a solvate thereof,

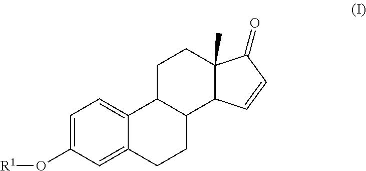

comprising the step of desulfinylation of a compound of formula (II) to produce compound of formula (I);

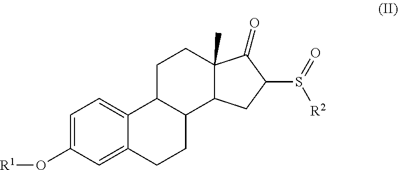

wherein:
R¹ is selected from the group comprising hydrogen, R³Si(R⁴)(R⁵)—, C₁₋₆alkyl, C₆₋₁₀aryl, C₆₋₁₀arylC₁₋₆alkyl, —CH₂—CH═CR^{a}R^{b}, and R⁶CO—; each of said C₁₋₆alkyl, C₆₋₁₀aryl, and C₆₋₁₀arylC₁₋₆alkyl may be optionally substituted with one or more substituents each independently selected from the group consisting of halo, C₁₋₆alkyl, haloC₁₋₆alkyl, C₁₋₆alkoxy, haloC₁₋₆alkoxy, C₆₋₁₀aryl, heterocyclyl, and nitro; preferably R¹ is selected from the group comprising R³Si(R⁴)(R⁵)—, C₁₋₆alkyl, C₆₋₁₀aryl, C₆₋₁₀arylC₁₋₆alkyl, and R⁶CO—; each of said C₁₋₆alkyl, C₆₋₁₀aryl, and C₆₋₁₀arylC₁₋₆alkyl may be optionally substituted with one or more substituents each independently selected from the group consisting of halo, C₁₋₆alkyl, haloC₁₋₆alkyl, C₁₋₆alkoxy, haloC₁₋₆alkoxy, and nitro; preferably R¹ is selected from the group comprising R³Si(R⁴)(R⁵)—, C₁₋₆alkyl, C₆₋₁₀aryl, and C₆₋₁₀arylC₁₋₆alkyl; preferably R¹ is R³Si(R⁴)(R⁵)—; preferably R¹ is selected from the group comprising R³Si(R⁴)(R⁵)—, C₁₋₄alkyl, C₆₋₁₀aryl, and C₆₋₁₀arylC₁₋₄alkyl; preferably R¹ is R³Si(R⁴)(R⁵)—;
R² is C₆₋₁₀aryl, or heteroaryl; wherein each C₆₋₁₀aryl or heteroaryl may be optionally substituted with one or more substituents each independently selected from the group consisting of halo, C₁₋₆alkyl, haloC₁₋₆alkyl, C₁₋₆alkoxy, haloC₁₋₆alkoxy, and nitro; preferably fluoro, chloro, bromo, nitro, C₁₋₄alkyl, C₁₋₄alkyloxy, or haloC₁₋₄alkyloxy; preferably, R² is C₆₋₁₀aryl, or 5-6 membered heteroaryl containing at least one N and/or S; wherein each C₆₋₁₀aryl or heteroaryl may be optionally substituted with one or more substituents each independently selected from the group consisting of halo, C₁₋₆alkyl, haloC₁₋₆alkyl, C₁₋₆alkoxy, haloC₁₋₆alkoxy, and nitro; preferably fluoro, chloro, bromo, nitro, C₁₋₄alkyl, C₁₋₄alkyloxy, or haloC₁₋₄alkyloxy; preferably, R² is C₆₋₁₀aryl, or 5-6 membered heteroaryl containing at least one N; wherein each C₆₋₁₀aryl or heteroaryl may be optionally substituted with one or more substituents each independently selected from the group consisting of halo, C₁₋₆alkyl, haloC₁₋₆alkyl, C₁₋₆alkoxy, haloC₁₋₆alkoxy, and nitro; preferably fluoro, chloro, bromo, nitro, C₁₋₄alkyl, C₁₋₄alkyloxy, or haloC₁₋₄alkyloxy;
R³, R⁴ and R⁵ are each independently C₁₋₆alkyl or C₆₋₁₀aryl, wherein each C₁₋₆alkyl or C₆₋₁₀aryl may be optionally substituted with one or more substituents each independently selected from the group consisting of halo, C₁₋₆alkyl, haloC₁₋₆alkyl, C₁₋₆alkoxy, and haloC₁₋₆alkoxy; preferably fluoro, chloro or C₁₋₄alkyl;
R⁶ is C₁₋₆alkyl or C₃₋₆cycloalkyl, wherein each C₁₋₆alkyl or C₃₋₆cycloalkyl may be optionally substituted by one or more substituents independently selected from the group consisting of halo, C₁₋₆alkyl, haloC₁₋₆alkyl, C₁₋₆alkoxy, and haloC₁₋₆alkoxy; preferably fluoro, chloro or C₁₋₄alkyl;
preferably wherein said desulfinylation step is performed by thermolysis.

In some embodiments R² is C₆₋₁₀aryl, or heteroaryl; wherein each C₆₋₁₀aryl or heteroaryl may be optionally substituted with one, two, three, four or five substituents; preferably R² is C₆₋₁₀aryl, or 5-6 membered heteroaryl containing at least one N and/or S; wherein each C₆₋₁₀aryl or heteroaryl may be optionally substituted with one, two, three, four of five substituents; preferably, R² is C₆₋₁₀aryl, or 5-6 membered heteroaryl containing at least one N; wherein each C₆₋₁₀aryl or heteroaryl may be optionally substituted with one, two, three, four or five substituents; preferably R² is phenyl, or pyridinyl; wherein each phenyl or pyridinyl may be optionally substituted with one, two, three, four or five substituents.

In some embodiments R² is C₆₋₁₀aryl, or heteroaryl; wherein each C₆₋₁₀aryl or heteroaryl may be optionally substituted with one or more substituents each independently selected from the group consisting of fluoro, chloro, bromo, nitro, C₁₋₄alkyl, C₁₋₄alkyloxy, haloC₁₋₄alkyloxy, phenyl, heterocyclyl, heteroaryl, —S(O)₂C₁₋₄alkyl, —S(O)₂, C₁₋₄haloalkyl, CO₂C₁₋₄alkyl, CO₂C₁₋₄haloalkyl, C(O)C₁₋₄alkyl, C(O)C₁₋₄haloalkyl, amino, mono-C₁₋₄alkylamino, di-C₁₋₄alkylamino, cyano; preferably, R² is C₆₋₁₀aryl, or 5-6 membered heteroaryl containing at least one N and/or S; wherein each C₆₋₁₀aryl or heteroaryl may be optionally substituted with one or more substituents each independently selected from the group consisting of fluoro, chloro, bromo, nitro, C₁₋₄alkyl, C₁₋₄alkyloxy, haloC₁₋₄alkyloxy, phenyl, heterocyclyl, heteroaryl, —S(O)₂C₁₋₄alkyl, —S(O)₂, C₁₋₄haloalkyl, CO₂C₁₋₄alkyl, CO₂C₁₋₄haloalkyl, C(O)C₁₋₄alkyl, C(O)C₁₋₄haloalkyl, amino, mono-C₁₋₄alkylamino, di-C₁₋₄alkylamino, cyano; preferably, R² is C₆₋₁₀aryl, or 5-6 membered heteroaryl containing at least one N; wherein each C₆₋₁₀aryl or heteroaryl may be optionally substituted with one or more substituents each independently selected from the group consisting of fluoro, chloro, bromo, nitro, C₁₋₄alkyl, C₁₋₄alkyloxy, haloC₁₋₄alkyloxy, phenyl, heterocyclyl, heteroaryl, —S(O)₂C₁₋₄alkyl, —S(O)₂, C₁₋₄haloalkyl, CO₂C₁₋₄alkyl, CO₂C₁₋₄haloalkyl, C(O)C₁₋₄alkyl, C(O)C₁₋₄haloalkyl, amino, mono-C₁₋₄alkylamino, di-C₁₋₄alkylamino, cyano; preferably R² is phenyl, or pyridinyl; wherein each phenyl or pyridinyl may be optionally substituted with one or more substituents each independently selected from the group consisting of fluoro, chloro, bromo, nitro, C₁₋₄alkyl, C₁₋₄alkyloxy, haloC₁₋₄alkyloxy, phenyl, heterocyclyl, heteroaryl, —S(O)₂C₁₋₄alkyl, —S(O)₂, C₁₋₄haloalkyl, CO₂C₁₋₄alkyl, CO₂C₁₋₄haloalkyl, C(O)C₁₋₄alkyl, C(O)C₁₋₄haloalkyl, amino, mono-C₁₋₄alkylamino, di-C₁₋₄alkylamino, cyano.

The present invention also encompasses a production line for preparing a compound of formula (I), preferably by desulfinylation of compound of formula (II), preferably by thermolysis, said production line comprising:
(a) sources of reactants including:
a source of compound of formula (II),
a source of solvent, and
optionally a source of at least one scavenger and optionally a source of at least one base, preferably at least one organic base;
(b) a solution tank in fluid communication with the sources of reactants,
(c) at least one pump bringing in fluid communication the solution tank with a reaction zone, preferably a thermolysis reaction zone, wherein
(d) the thermolysis reaction zone comprises:
a channel extending from an inlet to an outlet located downstream from the inlet, wherein the at least one pump is located directly upstream of the inlet,
a heating module arranged for heating the channel at a thermolysis temperature,
optionally a cooling zone for cooling the solution exiting the channel, said cooling zone being located downstream of the outlet, and
(e) optionally a separating zone for separating at least one compound of formula (I) from other thermolysis products.

The various sources may be provided with valves or volumetric pumps for controlling the flowrate of the various components from the corresponding sources to a solution tank. For example, piston pumps (or syringes) can be used to accurately control the flow of each reactant into the solution tank. The valves or volumetric pumps can be controlled by a controlling unit.

A reactive solution (reaction mixture) is formed in the solution tank composed of the reactants supplied from the sources of reactants. Preferably, the reactive solution comprises a compound of formula (II), a solvent, at least one scavenger and optionally at least one organic base. In some embodiments the reactive solution does not comprise a base. Optionally, the solution tank can be provided with a stirrer for enhancing homogeneity of the reactive solution. It may also be provided with heating means for heating the reactive solution, for example for lowering the viscosity of the reactive solution or to increase the solubility of the reagents.

Preferably, the solution tank is in fluid communication with at least one pump for injecting under pressure the reactive solution into an inlet of a channel extending from said inlet to an outlet located downstream from the inlet.

Pressures of up to 60.0 bar can be used for the thermolysis of the reactive solution, but lower pressures are preferred such as from 5.0 and 50.0 bar, preferably between 5.0 and 40.0 bar, preferably between 5.0 and 30.0 bar, preferably between 10.0 to 45.0 bar, preferably between 10.0 and 30.0 bar, preferably between 10.0 to 25.0 bar, preferably between 12.0 to 40.0 bar, preferably between 12.0 to 35.0 bar.

The channel preferably forms a capillary defined by a closed perimeter, Pe, and can be formed by a tube or by a recess in a plate. The channel can be equipped with a backpressure regulator, which can be inserted downstream to enable pressurized operation. Depending on the thermolysis temperature and type of heating module used, the channel can be made of stainless steel, preferably passivated stainless steel, aluminium, copper, PEEK, PEKK, PTFE, ceramics such as silicon carbide, and the like.

The channel can comprise a central portion comprised between the inlet and the outlet. If the channel is formed by a tube, the central portion can preferably form one or more coils. If it is formed by a recess in a plate, the recess may meander over the surface of the plate to form a serpentine. The inner walls of the channel can include specific structures, for instance to achieve high mixing. These configurations allow large linear lengths of channel to be housed in a small volume, thus saving space. The central portion of the channel can be part of a thermolysis microreactor module, which can comprise a heating module arranged for heating the channel at a thermolysis temperature of at least 100° C. The heating module can be a furnace of any type known in the art allowing the heating of the channel at said temperature and enclosing the central portion of the channel. For example, the heating module can be a conventional electric or gas furnace, or can heat the channel by radiation, such as IR, by induction, by Joule effect, and the like. The heating module must be suitable for heating the interior of the channel to a temperature of at least 100° C., preferably from 150 and 300° C., preferably between 20° and 275° C. If the reaction channel is formed by a recess in a plate, the heating module can also be integrated to the plate as a second recess independent of the channel, in which a thermofluid is circulated. The heating module can also be a heating cartridge hosted in the plate. Any heating means known in the art allowing the heating of the reaction mixture within a channel can be applied without affecting the present invention.

By controlling the pressure (and back pressure) in the channel, and depending on the inner hydraulic diameter, D, of the channel, the thermolysis time, t, can be controlled. The thermolysis time, t, can be comprised between 0.1 and 30 min, preferably between 0.5 to 25 min, preferably between 0.5 to 20 min, preferably from 1.0 and 15 min, preferably from 2 and 10 min, preferably from 2.5 and 8 min.

In order to control and build up a pressure inside the channel, a backpressure regulator BPR can be positioned downstream of the channel. The BPR can be a restriction in the channel cross-section, variable or not, or it can actively generate a counter-pressure.

The production line can further comprise a cooling module located directly downstream of the channel outlet, for cooling the reaction products of the thermolysis reaction. Any cooling means known in the art allowing the cooling of the reaction products in a short time can be applied without affecting the present invention. A cooling module is not mandatory, but is advantageous in case an in-line analysis module, such as an IR spectrometer, and the like, is provided for identifying the components flowing out of the channel. Cooling may be required as such in-line analysis modules may not be sufficiently temperature resistant to allow the reactants to flow through.

Optionally a separating module can be located downstream of the outlet for separating compound of formula (I) from other thermolysis products.

The separating module can be in-line or off-line with respect of the channel. The separation step may comprise neutralization, liquid-liquid extraction, liquid-liquid separation, gas-liquid separation, filtration on silica gel, in-line distillation and the like. In some embodiments, liquid-liquid extraction requires the injection of a secondary phase, such as an aqueous phase. The aqueous phase may contain an inorganic base. Alternatively, in-line liquid-liquid or gas-liquid separation can be carried out with a membrane or a settling tank.

A compound of formula (I) production line as described supra can be used for carrying out a continuous flow process according to the present invention for the production of compound of formula (I) by desulfinylation of compound of formula (II).

Preferably said continuous-flow process comprises the following steps:
(a) forming a mixture by contacting a compound of formula (II) with:
at least one solvent; optionally at least one scavenger; and optionally at least one organic base;
(b) feeding the mixture to an inlet of a channel of a thermolysis zone,
(c) exposing the mixture to thermolysis by driving a flow of the mixture along the channel from the inlet to an outlet, for a thermolysis time, t, at a pressure, P, and at a thermolysis temperature, T, greater than 100° C., to form thermolysis reaction products including at least a compound of formula (I);
(d) recovering the thermolysis reaction products at the outlet and
(e) optionally separating the at least one compound of formula (I) from the other thermolysis reaction products or keeping the compound of formula (I) in the reaction products.

As described herein above, pressure, P, in the channel can be controlled by the pressure, developed by at least one pump and the back-pressure developed by the backpressure regulator (BPR) if present. The pressure, P, can preferably range between 5 and 60 bar, preferably range between 5 and 50 bar, preferably range between 5 and 40 bar, preferably range between 8 and 60 bar, preferably range between 8 and 50 bar, preferably range between 8 and 40 bar, preferably range from 10 and 60 bar, preferably range between 10 and 50 bar, preferably range between 10 and 40 bar, preferably from 12 and 50 bar, more preferably between 12 to 40 bar, preferably between 13 to 35 bar, preferably range from 20 and 60 bar, preferably from 20 and 50 bar, preferably between 20 to 40 bar. The thermolysis time, t, can preferably be ranging from 0.5 to 45 min, preferably from 0.5 to 15 min, preferably from 0.5 to 10 min, preferably from 0.5 to 8.0 min, preferably from 1.0 to 30 min, preferably from 1.0 to 15 min, preferably from 1.0 to 8.0 min, preferably from 1.5 to 20 min, preferably from 2.0 to 15 min, preferably from 2.0 to 8.0 min, preferably from 2.5 to 6 min. The thermolysis temperature, T, can preferably range from 150 and 270° C., more preferably between 18° and 225° C.

The process can further comprise the steps of
(d1) feeding the reaction products from step (d) to an inlet of a channel of a cooling zone,
(d2) exposing the reaction products from step (d) to cooling by driving a flow of the reaction products along the channel from the inlet to an outlet, for a cooling time, tc, and at a cooling temperature, Tc; and
(d3) recovering the cooled reaction products at the outlet of the cooling zone, and optionally separating compound of formula (I) from other thermolysis reaction products.

The process can further comprise recovering the reaction products at the outlet of the reaction zone and contacting the reaction mixture comprising compound of formula (I) and other reaction products with an aqueous solution. Preferably, the aqueous solution comprises an inorganic base. More preferably, the aqueous solution comprises an inorganic base selected from the group comprising NaOH, NaHCO₃, K₂CO₃, NH₄Cl, Na₂S₂O₃, and mixture thereof, preferably said aqueous solution comprises NaOH. The aqueous solution is preferably a 0.5 mole/L to 2 mole/L NaOH solution, preferably a 1 mole/L NaOH solution.

Said reaction mixture can be contacted with said aqueous solution and stirred for at least 15 minutes, preferably at least 30 minutes to at most 2 hours.

In a preferred embodiment, the process comprises (d3) recovering the cooled reaction products at the outlet of the cooling zone, and contacting the cooled reaction mixture comprising compound of formula (I) and other reaction products with an aqueous solution, an aqueous solution comprising at least one inorganic base. More preferably, the aqueous solution comprises an inorganic base selected from the group comprising NaOH, NaHCO₃, K₂CO₃, NH₄Cl, Na₂S₂O₃, and mixture thereof, preferably said aqueous solution comprises NaOH. The aqueous solution is preferably a 0.5 mole/L to 2 mole/L NaOH solution, preferably a 1 mole/L NaOH solution.

The skilled artisan knows how to convert the disclosed parameters and conditions from small-scale conditions (R&D laboratory) to industrial-scale conditions.

The process can further comprise removing the organic layer from the mixture comprising the aqueous solution, and evaporating, preferably under reduced pressure the organic layer to recover the reaction products.

The present invention also encompasses the composition comprising said reaction products.

Preferably said composition comprises a compound of formula (I) and at least one additional by-product selected from the group comprising a compound of formula (IIa), (IIb), (IIc) and (IId) and mixture thereof;

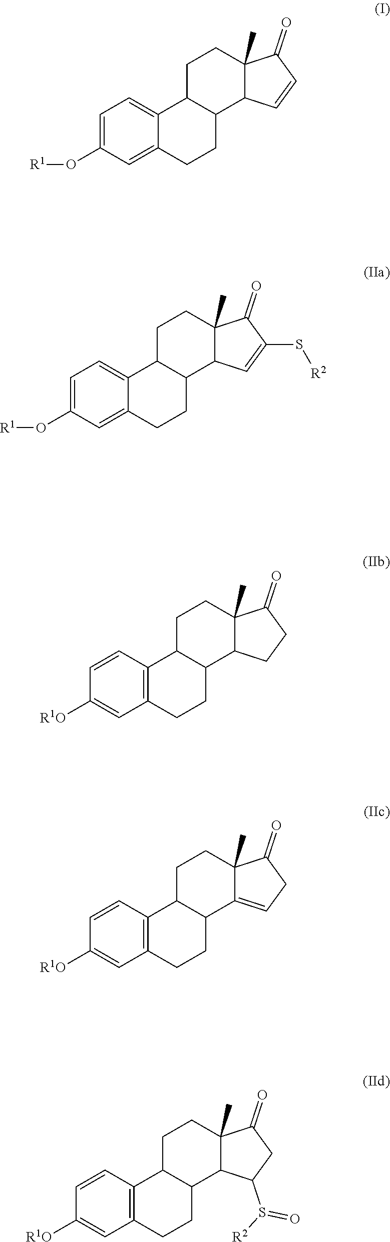

wherein
R¹ is selected from the group comprising hydrogen, R³Si(R⁴)(R⁵)—, C₁₋₆alkyl, C₆₋₁₀aryl, C₆₋₁₀arylC₁₋₆alkyl, —CH₂—CH═CR^{a}R^{b}, C₁₋₆alkoxy, C₃₋₆cycloalkyl, and R⁶CO—; each of said C₁₋₆alkyl, C₆₋₁₀aryl, C₆₋₁₀arylC₁₋₆alkyl, and C₃₋₆cycloalkyl may be optionally substituted with one or more substituents each independently selected from the group consisting of halo, C₁₋₆alkyl, haloC₁₋₆alkyl, C₁₋₆alkoxy, haloC₁₋₆alkoxy, C₆₋₁₀aryl, heterocyclyl, and nitro;
R² is C₆₋₁₀aryl, or heteroaryl; wherein each C₆₋₁₀aryl or heteroaryl may be optionally substituted with one or more substituents each independently selected from the group consisting of halo, C₁₋₆alkyl, haloC₁₋₆alkyl, C₁₋₆alkoxy, haloC₁₋₆alkoxy, C₂₋₆alkenyl, C₂₋₆alkynyl, C₆₋₁₀aryl, heterocyclyl, heteroaryl, hydroxyl, —S(O)₂R⁷, —S(O)R⁸, —CO₂R⁹, —C(O)R¹⁰, —SR¹³, —C(O)SR¹⁴, NR¹¹R¹², cyano and nitro; preferably fluoro, chloro, bromo, nitro, C₁₋₄alkyl, C₁₋₄alkyloxy, or haloC₁₋₄alkyloxy;
R⁶ is C₁₋₆alkyl, C₆₋₁₀aryl, or C₃₋₆cycloalkyl, wherein each C₁₋₆alkyl, C₆₋₁₀aryl, or C₃₋₆cycloalkyl may be optionally substituted by one or more substituents independently selected from the group consisting of halo, C₁₋₆alkyl, haloC₁₋₆alkyl, C₁₋₆alkoxy, and haloC₁₋₆alkoxy;
R⁷, R⁸, R⁹ and R¹⁰ are each independently selected from the group consisting of hydrogen, hydroxyl, C₁₆alkyl, C₆₁₀aryl, and C₃₋₆cycloalkyl, wherein each C₁₋₆alkyl, C₆₋₁₀aryl, or C₃₋₆cycloalkyl may be optionally substituted by one or more substituents independently selected from the group consisting of halo, C₁₋₆alkyl, haloC₁₋₆alkyl, C₁₋₆alkoxy, and haloC₁₋₆alkoxy;
R¹¹ and R¹² are each independently selected from the group consisting of hydrogen, C₁₋₆alkyl, C₆₋₁₂aryl, C₃₋₆cycloalkyl; wherein each C₁₋₆alkyl, C₆₋₁₀aryl, or C₃₋₆cycloalkyl may be optionally substituted by one or more substituents independently selected from the group consisting of halo, C₁₋₆alkyl, haloC₁₋₆alkyl, C₁₋₆alkoxy, and haloC₁₋₆alkoxy;
R¹³ and R¹⁴ are each independently selected from the group consisting of hydrogen, C₁₋₆alkyl, C₆₋₁₀aryl, and C₃₋₆cycloalkyl, wherein each C₁₋₆alkyl, C₆₋₁₀aryl, or C₃₋₆cycloalkyl may be optionally substituted by one or more substituents independently selected from the group consisting of halo, C₁₋₆alkyl, haloC₁₋₆alkyl, C₁₋₆alkoxy, and haloC₁₋₆alkoxy;
R^{a} and R^{b} are each independently hydrogen, C₁₋₆alkyl or C₆₋₁₀aryl.

Preferably said composition comprises a compound of formula (I) and at least one additional by-product selected from the group comprising a compound of formula (IIa), (IIb), (IIc) and (IId) and mixture thereof;

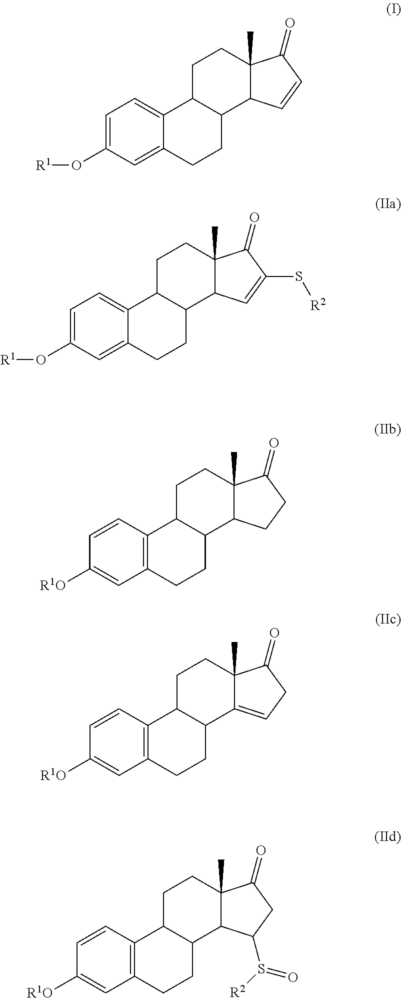

wherein
R¹ is selected from the group comprising hydrogen, R³Si(R⁴)(R⁵)—, C₁₋₆alkyl, C₆₋₁₀aryl, C₆₋₁₀arylC₁₋₆alkyl, —CH₂—CH═CR^{a}R^{b}, and R⁶CO—; each of said C₁₋₆alkyl, C₆₋₁₀aryl, and C₆₋₁₀arylC₁₋₆alkyl may be optionally substituted with one or more substituents each independently selected from the group consisting of halo, C₁₋₆alkyl, haloC₁₋₆alkyl, C₁₋₆alkoxy, haloC₁₋₆alkoxy, C₆₋₁₀aryl, heterocyclyl, and nitro;
R² is C₆₋₁₀aryl, or heteroaryl; wherein each C₆₋₁₀aryl or heteroaryl may be optionally substituted with one or more substituents each independently selected from the group consisting of halo, C₁₋₆alkyl, haloC₁₋₆alkyl, C₁₋₆alkoxy, haloC₁₋₆alkoxy, and nitro; preferably fluoro, chloro, bromo, nitro, C₁₋₄alkyl, C₁₋₄alkyloxy, or haloC₁₋₄alkyloxy;
R⁶ is C₁₋₆alkyl, C₆₋₁₀aryl, or C₃₋₆cycloalkyl, wherein each C₁₋₆alkyl, C₆₋₁₀aryl, or C₃₋₆cycloalkyl may be optionally substituted by one or more substituents independently selected from the group consisting of halo, C₁₋₆alkyl, haloC₁₋₆alkyl, C₁₋₆alkoxy, and haloC₁₋₆alkoxy;
R^{a} and R^{b} are each independently hydrogen, C₁₋₆alkyl or C₆₋₁₀aryl.

The present invention also encompasses a composition comprising a compound of formula (I), and a compound of formula (IIa) as defined herein.

In some embodiments, the composition comprises a compound of formula (I), and compound of formula (IIa), and optionally any one of compounds of formula (IIb), (IIc), (IId) or a mixture thereof.

In some embodiments, the composition comprises a compound of formula (I), and compound of formula (IIb), and optionally any one of compounds of formula (IIa), (IIc), (IId) or mixture thereof.

In some embodiments, the composition comprises a compound of formula (I), and a compound of formula (IIc), and optionally any one of compounds of formula (IIa), (IIb), (IId) or a mixture thereof.

In some embodiments, the composition comprises an amount of at least 0.01% area by HPLC of compound (IIa), based on the total area of the composition as determined by HPLC; preferably an amount of at least 0.1% area by HPLC of compound (IIa); preferably an amount of 0.5% area by HPLC of compound (IIa); preferably an amount of at least 1.0% area by HPLC of compound (IIa).

In some embodiments, the composition comprises an amount of at most 25.0% area by HPLC of compound (IIa), based on the total area of the composition as determined by HPLC; preferably an amount of at most 20.0% of compound (IIa); preferably an amount of at most 15.0% area by HPLC of compound (IIa); preferably an amount of at most 10.5% area by HPLC of compound (IIa); preferably an amount of at most 5.0% area by HPLC of compound (IIa).

In some embodiments, the composition comprises an amount of at most 16.0% area by HPLC of compound (IIb), based on the total area of the composition as determined by HPLC; preferably an amount of at most 10.0% area by HPLC of compound (IIb); preferably an amount of at most 5.0% area by HPLC of compound (IIb).

In some embodiments, the composition comprises an amount of at most 10.0% area by HPLC of compound (IIc), based on the total area of the composition as determined by HPLC; preferably an amount of at most 8.0% area by HPLC of compound (IIc); preferably an amount of at most 5.0% area by HPLC of compound (IIc).

In some embodiments, the composition comprises an amount of at most 3.0% area by HPLC of compound (IIb), based on the total area of the composition as determined by HPLC; preferably an amount of at most 2.0% area by HPLC of compound (IIb); preferably an amount of at most 1.0% area by HPLC of compound (IIb).

The following examples are provided for the purpose of illustrating the present invention and by no means should be interpreted to limit the scope of the present invention.

### EXAMPLES

The following apparatus and reactants were used for the continuous-flow process used in the examples.

### Pump

Knauer AZURA P 4.1S HPLC pumps or Chemyx Fusion 6000High Force syringe pumps equipped with stainless steel syringes (20 mL) with Dupont Kalrez Spectrum AS-568 O-rings (0.549×0.103″) were used to handle the liquid feeds.

### Stainless Steel (SS) Coil Reactors

SS coil reactors were constructed with deburred-end, steam-cleaned and acid-passivated 316 SS tubing ([1.59 mm outer diameter, 0.76 mm internal diameter] or [3.17 mm outer diameter, 2.10 mm internal diameter]) of defined internal volumes.

### PFA Tubing and Coils

Perfluoroalkoxy (PFA) coil reactors and collection lines were constructed from PFA tubing (high purity PFA; 1.58 mm outer diameter, 750 μm internal diameter).

### Connectors, Ferrules and Mixers

Sections of the reactor that were not subjected to high temperatures were equipped with coned poly-ether-ether ketone (PEEK) fittings and micromixers. Sections of the reactor that were subjected to high temperatures were equipped with Valco SS fittings, ferrules and unions. Connectors, ferrules and unions were purchased from IDEX/Upchurch.

### Check-Valves

The check-valves inserted between the pumps and the reactors were purchased from IDEX/Upchurch Scientific (PEEK or SS check-valve holder).

### Back-Pressure Regulator

Spring loaded BPRs were purchased from IDEX/Upchurch Scientific (PEEK or SS check-valve holder).

### Thermoregulatory Devices

1/16″ SS coils were thermoregulated in a ThalesNano Phoenix Flow Reactor™. ⅛″ SS coils were thermoregulated in a modified GC oven.

### Chemicals and Solvents

All solvents used were purchased from Fisher Scientific.

NaHCO₃, NaOH and HCl were purchased from VWR.

Imidazole, tert-butyldimethylsilyl chloride and methyl benzenesulfinate were purchased from TCI. NaH, sodium borohydride, cerium chloride heptahydrate and triethylamine were purchased from Sigma Aldrich.

Et₃N was distilled prior to use and stored at room temperature.

Dimethyl acetylenedicarboxylate (DMAD) was distilled prior to use and stored at 4° C.

CH₂Cl₂, DMSO and MeOH were dried under molecular sieves (3 Å for MeOH and 4 Å for CH₂Cl₂ and DMSO).

All other reagents and solvents were purchased from Sigma Aldrich and were used as supplied.

### HPLC Method:

Eluent: A: Water; B: Acetonitrile

Gradient of the eluents are indicated in Table 1.

TABLE 1

| Time [min] | A [%] | B [%] |
|---|---|---|
| 0 | 50 | 50 |
| 3 | 50 | 50 |
| 15 | 0 | 100 |
| 25 | 0 | 100 |
| 27 | 50 | 50 |
| 35 | 50 | 50 |

Flow rate: 2 mL/min-1

Column: C18, 100×4.6 mm, 3 μm

Oven Temperature: 40° C.

Diode Array Detector: 180-800 nm

Wavelength for analysis: 190 nm

### Example 1: Synthesis of 3-tert-butyldimethylsilyloxy-estra-1,3,5(10)-15-tetraene-17-one (compound 1)

Compound 1 was prepared as schematically illustrated in scheme 1.

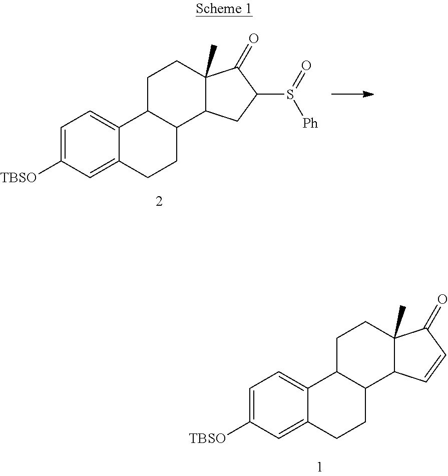 [SMILES: CC(C)(C)[Si](C)(C)Oc1ccc2c(c1)CCC1C2CC[C@]2(C)C(=O)C(S(=O)c3ccccc3)CC12 ; CC(C)(C)[Si](C)(C)Oc1ccc2c(c1)CCC1C2CC[C@]2(C)C(=O)C=CC12]

To a solution of 3-tert-butyldimethylsilyloxy-16-(phenylsulfinyl)-estra-1,3,5(10)-triene-17-one (2) (9.71 g, 19.08 mmol) in toluene (83 mL) at room temperature were successively added Et₃N (0.3 mL, 1.90 mmol) and dimethyl acetylene-dicarboxylate (DMAD) (3.5 mL, 28.52 mmol). The solution was pumped with a flow rate of 0.833 mL/min (for a thermolysis time of 6 minutes) into a stainless-steel tubing heated at 210° C. The feed was maintained liquid with a back-pressure regulator of 34 bar (500 psi) then cooled to 0° C. in a PFA coil. The entirety of the crude product was collected in a round bottom flask and stirred with a 1 M NaOH solution (80 mL) for 30 min. The organic layer was separated and evaporated under reduced pressure to afford the compound (1) as a solid.

¹H-NMR (CDCl₃): δ 0.20 (s, 6H, (CH₃)₂—Si—), 1.00 (s, 9H, (CH₃)₃—C—Si—), 1.13 (s, 3H, CH₃ at C-18), 1.20-2.70 (m, 11H), 2.80-3.00 (m, 2H), 6.10 (dd, 1H, H15), 6.58 (broad s, 1 H, H4), 6.62 (dd, 1H, H2), 7.11 (d, 1H, H1), 7.63 (dd, 1H, H16).

### Example 2: Synthesis of 3-tert-butyldimethylsilyloxy-estra-1,3,5(10)-15-tetraene-17-One (Compound 1) in Different Solvents and at Different Thermolysis Temperatures

3-tert-Butyldimethylsilyloxy-estra-1,3,5(10)-15-tetraene-17-one (1) was prepared from a 0.06 M solution of 3-tert-butyldimethylsilyloxy-16-(phenylsulfinyl)-estra-1,3,5(10)-triene-17-one (2) in different solvents and thermolysis temperatures as listed in Table 2, using dimethyl acetylene-dicarboxylate (1.0 equiv.) and trimethyl phosphite (1.0 equiv.), following the procedure and set up described in Example 1.

Table 2 shows the yield of the desulfinylation reaction using different thermolysis temperatures and solvents. The yields are given in % area as determined by HPLC.

TABLE 2

| Solvent | 190° C./15 min | 200° C./10 min | 210° C./5 min |
|---|---|---|---|
| Xylene | 81% | n.d. | 74% |
| Toluene | 72% | 76% | 76% |
| Anisole | 76% | 76% | 78% |
| Cl-benzene | 81% | 78% | 80% |
| 2-Me-THF | 75% | 78% | 76% |
| AcOEt | 78% | 78% | 78% |
| Dichloroethane | n.d. | n.d. | 77% |
| CHCl₃ | n.d. | n.d. | 79% |

n.d. not determined

### Example 3: Synthesis of 3-tert-butyldimethylsilyloxy-estra-1,3,5(10)-15-tetraene-17-One (Compound 1) Using Different Thermolysis Times and Different Thermolysis Temperatures

3-tert-Butyldimethylsilyloxy-estra-1,3,5(10)-15-tetraene-17-one (compound 1) was prepared from a 0.06 M solution of 3-tert-butyldimethylsilyloxy-16-(phenylsulfinyl)-estra-1,3,5(10)-triene-17-one (2) in o-xylene using trimethyl phosphite (3.0 equiv.), at different thermolysis temperatures and thermolysis times as listed in Table 3, following the procedure and set up described in Example 1.

Table 3 shows the yield of the desulfinylation reaction varying the thermolysis temperatures and thermolysis time. The yields are given in % area as determined by HPLC.

TABLE 3

| Thermolysis time | Temperature (° C.) | | | | |
|---|---|---|---|---|---|
| (min) | 180 | 190 | 200 | 210 | 220 |
| 5 | 25% | 39% | 48% | 47% | 43% |
| 10 | 43% | 53% | 52% | 44% | 38% |
| 15 | 53% | 52% | 47% | 41% | 33% |

### Example 4: Synthesis of 3-tert-butyldimethylsilyloxy-estra-1,3,5(10)-15-tetraene-17-One (Compound 1) Using Different Scavengers

3-tert-Butyldimethylsilyloxy-estra-1,3,5(10)-15-tetraene-17-one compound of formula (I) (compound 1) was prepared from a 0.06 M solution of 3-tert-butyldimethylsilyloxy-16-(phenylsulfinyl)-estra-1,3,5(10)-triene-17-one (2) in toluene in the presence of different scavengers as listed in Table 4, following the procedure described in Example 1.

Table 4 shows the yield of the desulfinylation reaction in the presence of different scavengers. The yields are given in % area as determined by HPLC.

TABLE 4

| | Amount of | |
|---|---|---|
| Scavenger | scavenger (equiv.) | Yield (%) |
| 1,4-dimethoxybenzene | 2.0 | 74 |
| 2,6-lutidine | 1.0 | 64 |
| Crotonaldehyde | 2.0 | 25 |
| Cyclohexanone | 2.0 | 42 |
| DABCO | 2.0 | 72 |
| DEF | 1.0 | 57 |
| DEtAD | 1.0 | 62 |
| DIAD | 2.0 | 42 |
| DMAD | 1.0 | 78 |
| MBT | 1.0 | 45 |
| Methyl propiolate | 2.0 | 61 |
| Et₃N | 2.0 | 76 |
| Phenylacetylene | 2.0 | 40 |
| TEMPO | 2.0 | 84 |
| TMP | 3.0 | 52 |

### Example 5: Synthesis of 3-tert-butyldimethylsilyloxy-estra-1,3,5(10)-15-tetraene-17-One (Compound 1) Using Different Bases at Different Concentrations

3-tert-Butyldimethylsilyloxy-estra-1,3,5(10)-15-tetraene-17-one (compound 1) was prepared from a 0.22 M solution of 3-tert-butyldimethylsilyloxy-16-(phenylsulfinyl)-estra-1,3,5(10)-triene-17-one (2) in toluene in the presence of dimethyl acetylene-dicarboxylate (1.5 eq.) and different bases as listed in Table 5, following the procedure and set up described in Example 1.

Table 5 shows the yield of the desulfinylation reaction in the presence of different bases. The yields are given in % area as determined by HPLC.

TABLE 5

| | Amount | | |
|---|---|---|---|
| Base | 0.1 equiv. | 0.3 equiv. | 0.5 equiv. |
| None | 77% | | |
| DBU | 90% | 88% | 82% |
| DABCO | n.d. | 84% | n.d. |
| Et₃N | 85% | 86% | 87% |
| DIPEA | n.d. | 83% | n.d. |
| TEMPO | n.d. | 84% | n.d. |
| Imidazole | 81% | 83% | 85% |
| 2,6-lutidine | n.d. | 81% | n.d. |

### Example 6: Synthesis of 3-tert-butyldimethylsilyloxy-estra-1,3,5(10)-15-tetraene-17-One (Compound 1), Identification and Quantification of Side Products

3-tert-Butyldimethylsilyloxy-estra-1,3,5(10)-15-tetraene-17-one (compound 1) was prepared from 3-tert-butyldimethylsilyloxy-16-(phenylsulfinyl)-estra-1,3,5(10)-triene-17-one (2), following the procedure and set up described in Example 1, applying the conditions listed in Table 6. The products of these reactions were analyzed using HPLC, NMR, IR spectroscopy and mass spectrometry. The following impurities were identified:

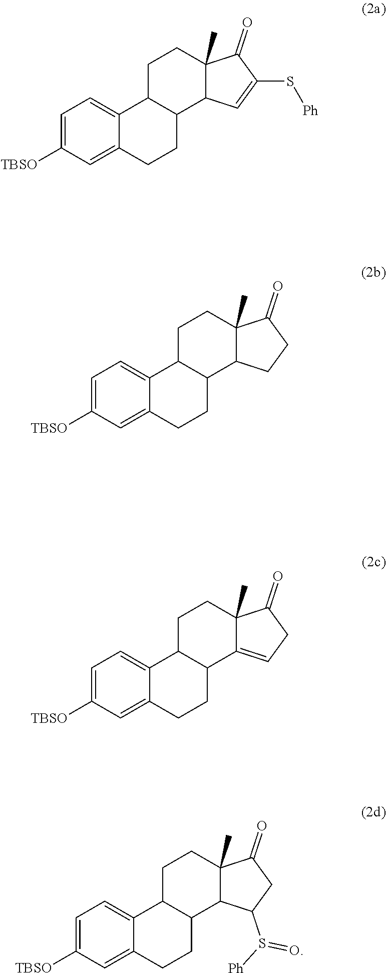 [SMILES: CC(C)(C)[Si](C)(C)Oc1ccc2c(c1)CCC1C2CC[C@]2(C)C(=O)C(Sc3ccccc3)=CC12 ; CC(C)(C)[Si](C)(C)Oc1ccc2c(c1)CCC1C2CC[C@]2(C)C(=O)CCC12 ; CC(C)(C)[Si](C)(C)Oc1ccc2c(c1)CCC1C3=CCC(=O)[C@@]3(C)CCC21 ; CC(C)(C)[Si](C)(C)Oc1ccc2c(c1)CCC1C2CC[C@]2(C)C(=O)CC(S(=O)c3ccccc3)C12]

Table 6 shows the % area by HPLC of compound (1) and by-products (2a), (2b), (2c), and (2d) obtained depending on the experimental conditions used.

TABLE 6

| | Scavenger | Scavenger | Base | Comp-2 | | 1 | 2b | 2c | 2a | 2d |
|---|---|---|---|---|---|---|---|---|---|---|
| Scavenger | Equiv. | Base | Equiv. | (M) | Solvent | % area | % area | % area | % area | % area |
| DMAD | 1.5 | Et3N | 0.1 | 0.22 | Toluene | 91 | 3 | 5 | 1 | 0 |
| DMAD | 1.5 | DBU | 0.05 | 0.22 | Toluene | 90 | 5 | 2 | 3 | 0 |
| DMAD | 1.5 | DBU | 0.1 | 0.22 | Toluene | 88 | 5 | 3 | 4 | 0 |
| DMAD | 1.5 | Et3N | 0.1 | 0.22 | Toluene | 88 | 5 | 4 | 3 | 0 |
| DMAD | 1.5 | Et3N | 0.1 | 0.22 | Toluene | 88 | 5 | 4 | 4 | 0 |
| DMAD | 1.5 | Et3N | 0.2 | 0.22 | Toluene | 87 | 5 | 4 | 4 | 0 |
| DMAD | 1.5 | Et3N | 0.1 | 0.22 | Toluene | 87 | 4 | 4 | 5 | 0 |
| DMAD | 1.5 | Et3N | 0.05 | 0.22 | Toluene | 86 | 4 | 5 | 4 | 0 |
| DMAD | 1.5 | Et3N | 0.1 | 0.22 | Toluene | 86 | 4 | 5 | 5 | 0 |
| DMAD | 1.5 | Et3N | 0.1 | 0.22 | Toluene | 86 | 5 | 5 | 4 | 0 |
| DMAD | 1.5 | Et3N | 0.1 | 0.22 | Toluene | 86 | 4 | 4 | 6 | 0 |
| DMAD | 1.5 | imidazole | 0.2 | 0.22 | Toluene | 85 | 7 | 1 | 7 | 0 |
| DMAD | 1.5 | Et3N | 0.05 | 0.22 | Toluene | 85 | 5 | 3 | 4 | 0 |
| DMAD | 1.5 | Et3N | 0.1 | 0.22 | Toluene | 85 | 6 | 5 | 4 | 0 |
| DMAD | 1.5 | Et3N | 0.1 | 0.22 | Toluene | 85 | 2 | 1 | 3 | 0 |
| DMAD | 1.5 | Et3N | 0.1 | 0.22 | Toluene | 85 | 4 | 2 | 4 | 0 |
| DMAD | 1.5 | Et3N | 0.1 | 0.22 | Toluene | 85 | 10 | 2 | 4 | 0 |
| TEMPO | 2 | / | / | 0.06 | Toluene | 84 | 12 | 3 | 1 | 0 |
| DMAD | 1.5 | Et3N | 0.1 | 0.22 | Toluene | 84 | 10 | 2 | 4 | 0 |
| DMAD | 1.5 | Et3N | 0.01 | 0.22 | Toluene | 84 | 4 | 4 | 7 | 0 |
| DMAD | 1.5 | TEMPO | 0.1 | 0.22 | Toluene | 84 | 4 | 5 | 7 | 0 |
| DMAD | 1.5 | Et3N | 0.1 | 0.22 | Toluene | 84 | 6 | 5 | 6 | 0 |
| DMAD | 1 | / | 1 | 0.06 | Toluene | 83 | 5 | 0 | 11 | 1 |
| TEMPO | 1 | / | / | 0.22 | Toluene | 83 | 11 | 4 | 2 | 0 |
| DMAD | 1.5 | / | / | 0.22 | Toluene | 83 | 4 | 3 | 10 | 0 |
| DMAD | 1.5 | / | / | 0.22 | Toluene | 83 | 4 | 3 | 10 | 0 |
| DMAD | 1.5 | imidazole | 0.1 | 0.22 | Toluene | 83 | 6 | 4 | 7 | 0 |
| DMAD | 1.5 | DIPEA | 0.1 | 0.22 | Toluene | 83 | 5 | 6 | 6 | 0 |
| DMAD | 1.5 | Et3N | 0.025 | 0.22 | Toluene | 83 | 4 | 8 | 5 | 0 |
| TMP | 1 | DMAD | 1 | 0.06 | o-Xylene | 82 | 6 | 1 | 0 | 0 |
| DMAD | 1.5 | DBU | 0.2 | 0.22 | Toluene | 82 | 7 | 4 | 4 | 0 |
| DMAD | 1.5 | Et3N | 0.1 | 0.22 | Toluene | 82 | 5 | 6 | 7 | 0 |
| DMAD | 1.5 | DBU | 0.025 | 0.22 | Toluene | 82 | 6 | 6 | 6 | 0 |
| DMAD | 1.5 | Et3N | 0.1 | 0.22 | Toluene | 82 | 2 | 5 | 11 | 0 |
| TMP | 1 | DMAD | 1 | 0.06 | Cl-benzene | 81 | 8 | 6 | 5 | 0 |
| DMAD | 2 | 2,6-lutidine | 2 | 0.06 | Toluene | 81 | 4 | 0 | 15 | 0 |
| Et3N | 1 | TEMPO | 2 | 0.06 | Toluene | 81 | 12 | 4 | 2 | 1 |
| DMAD | 1 | TEMPO | 1 | 0.22 | Toluene | 81 | 7 | 4 | 4 | 2 |
| TEMPO | 1 | / | 1 | 0.36 | Toluene | 81 | 7 | 10 | 2 | 0 |
| DMAD | 1.5 | / | / | 0.22 | Toluene | 81 | 3 | 2 | 5 | 0 |
| DMAD | 1.5 | imidazole | 0.1 | 0.22 | Toluene | 81 | 5 | 6 | 8 | 0 |
| DMAD | 1.5 | 2,6-lutidine | 0.1 | 0.22 | Toluene | 81 | 6 | 5 | 8 | 0 |
| DMAD | 1.5 | Et3N | 0.1 | 0.22 | Toluene | 81 | 3 | 3 | 13 | 0 |
| DMAD | 1.5 | Et3N | 0.1 | 0.22 | Toluene | 81 | 9 | 2 | 8 | 0 |
| TMP | 1 | DMAD | 1 | 0.06 | o-Xylene | 80 | 8 | 3 | 8 | 0 |
| DMAD | 2 | 2,6-lutidine | 1 | 0.06 | Toluene | 80 | 3 | 0 | 17 | 0 |
| Et3N | 2 | TEMPO | 1 | 0.22 | Toluene | 80 | 8 | 3 | 1 | 1 |
| DMAD | 1 | / | 1 | 0.11 | Toluene | 79 | 5 | 5 | 11 | 0 |
| DMAD | 1 | 2,6-lutidine | 2 | 0.06 | Toluene | 79 | 9 | 4 | 7 | 0 |
| TMP | 1 | DMAD | 1 | 0.06 | CHCl3 | 79 | 11 | 0 | 7 | 0 |
| DMAD | 1.5 | / | 1 | 0.22 | Toluene | 79 | 5 | 6 | 10 | 0 |
| DMAD | 1.5 | Et3N | 0.5 | 0.22 | Toluene | 79 | 6 | 7 | 8 | 0 |
| DMAD | 1.5 | DBU | 0.1 | 0.22 | Toluene | 79 | 10 | 1 | 4 | 0 |
| DMAD | 1 | / | / | 0.06 | o-Xylene | 78 | 5 | 1 | 14 | 0 |
| TMP | 1 | DMAD | 1 | 0.06 | Anisole | 78 | 9 | 3 | 9 | 0 |
| DMAD | 1 | 2,6-lutidine | 1 | 0.06 | Toluene | 78 | 8 | 6 | 8 | 0 |
| DMAD | 1.5 | / | / | 0.22 | Toluene | 78 | 4 | 3 | 10 | 0 |
| TMP | 1 | DMAD | 1 | 0.06 | o-Xylene | 77 | 10 | 4 | 4 | 0 |
| TMP | 1 | DMAD | 1 | 0.06 | 2-Me-THF | 77 | 11 | 3 | 8 | 0 |
| DMAD | 1 | 2,6-lutidine | 1 | 0.06 | Toluene | 77 | 8 | 0 | 10 | 2 |
| DMAD | 1.5 | / | / | 0.22 | Toluene | 77 | 4 | 0 | 13 | 0 |
| TMP | 1 | DMAD | 1 | 0.06 | DCE | 77 | 12 | 2 | 6 | 0 |
| DMAD | 1 | / | 1 | 0.06 | Toluene | 76 | 5 | 4 | 13 | 0 |
| DMAD | 1 | Pyridine | 1 | 0.06 | Toluene | 76 | 5 | 7 | 11 | 0 |
| DMAD | 3 | 2,6-lutidine | 3 | 0.22 | Toluene | 76 | 5 | 5 | 10 | 0 |
| Et3N | 2 | / | 1 | 0.06 | Toluene | 76 | 12 | 6 | 1 | 0 |
| DMAD | 1.25 | / | 1 | 0.22 | Toluene | 76 | 5 | 1 | 14 | 0 |
| DMAD | 1.5 | / | 1 | 0.22 | Toluene | 76 | 7 | 4 | 4 | 0 |
| TMP | 1 | DMAD | 1 | 0.06 | EtOAc | 75 | 12 | 2 | 6 | 0 |
| DMAD | 1 | / | 1 | 0.06 | Toluene | 75 | 6 | 4 | 15 | 0 |
| DMAD | 1 | MeOH | 0.5 | 0.06 | Toluene | 75 | 5 | 3 | 17 | 0 |
| DMAD | 1 | / | / | 0.36 | DCE | 75 | 4 | 2 | 5 | 0 |
| DMAD | 1.5 | / | / | 0.57 | DCE | 75 | 5 | 7 | 7 | 0 |
| DMAD | 1.1 | / | / | 0.22 | Toluene | 75 | 4 | 1 | 12 | 0 |
| DMAD | 1.5 | / | 1 | 0.22 | Toluene | 75 | 9 | 0 | 7 | 0 |
| DMAD | 1.5 | DBU | 0.01 | 0.22 | Toluene | 75 | 9 | 2 | 2 | 0 |
| TMP | 3 | DMAD | 1 | 0.06 | Toluene | 74 | 10 | 0 | 16 | 0 |
| 1,4-dimethoxybenzene | 2 | 1 | / | 0.06 | Toluene | 74 | 14 | 3 | 7 | 2 |
| DMAD | 1.5 | / | / | 0.22 | Toluene | 74 | 12 | 2 | 7 | 0 |
| TMP | 1 | DMAD | 1 | 0.06 | o-Xylene | 74 | 8 | 0 | 1 | 0 |
| DMAD | 3 | 2,6-lutidine | 3 | 0.23 | Toluene | 73 | 5 | 4 | 14 | 0 |
| DMAD | 3 | / | / | 0.22 | Toluene | 73 | 3 | 1 | 18 | 0 |
| DABCO | 2 | / | / | 0.06 | Toluene | 72 | 11 | 8 | 9 | 0 |
| DMAD | 1 | 1 | 1 | 0.56 | DCE | 72 | 6 | 14 | 8 | 0 |
| DMAD | 1.5 | 1 | / | 0.22 | Toluene | 72 | 0 | 2 | 7 | 0 |
| TMP | 1 | DMAD | 1 | 0.06 | Toluene | 72 | 6 | 0 | 8 | 0 |
| DMAD | 1.5 | / | / | 0.06 | o-Xylene | 71 | 13 | 3 | 13 | 0 |
| DMAD | 1 | / | / | 0.06 | Toluene | 71 | 5 | 6 | 18 | 0 |
| DIPEA | 2 | 1 | / | 0.06 | Toluene | 71 | 9 | 5 | 0 | 0 |
| DMAD | 1.5 | / | 1 | 0.22 | Toluene | 71 | 3 | 3 | 7 | 0 |
| TMP | 1 | DMAD | 1 | 0.06 | xylene | 70 | 10 | 5 | 10 | 0 |
| DMAD | 1 | 1 | 1 | 0.18 | Toluene | 70 | 6 | 3 | 12 | 1 |
| DMAD | 3 | / | / | 0.36 | Toluene | 70 | 4 | 4 | 21 | 0 |
| DMAD | 3 | 1 | / | 0.29 | Toluene | 70 | 3 | 3 | 21 | 0 |
| Et3N | 5 | 1 | / | 0.22 | Toluene | 70 | 12 | 10 | 0 | 0 |
| Et3N | 5 | / | 1 | 0.22 | Toluene | 69 | 12 | 10 | 0 | 0 |
| DMAD | 1 | Pyridine | 2 | 0.06 | Toluene | 68 | 3 | 0 | 11 | 0 |
| Et3N | 1 | / | 1 | 0.22 | Toluene | 68 | 12 | 10 | 0 | 0 |
| TEMPO | 1 | / | / | 0.36 | DCE | 68 | 7 | 11 | 4 | 0 |
| 2,6-lutidine | 1.5 | / | / | 0.06 | o-Xylene | 67 | 16 | 6 | 9 | 1 |
| DMAD | 1 | / | / | 0.06 | Toluene | 67 | 5 | 6 | 21 | 0 |
| DMAD | 5 | 2,6-lutidine | 5 | 0.28 | Toluene | 67 | 5 | 7 | 6 | 0 |
| DMAD | 1.5 | DABCO | 0.1 | 0.22 | Toluene | 67 | 5 | 6 | 10 | 0 |
| Et3N | 3 | / | 1 | 0.22 | Toluene | 65 | 12 | 15 | 0 | 0 |
| Et3N | 3 | / | / | 0.22 | Toluene | 65 | 12 | 14 | 0 | 0 |
| DMAD | 1.5 | / | 1 | 0.22 | Toluene | 65 | 2 | 14 | 19 | 0 |
| DMAD | 1 | / | 1 | 0.22 | Toluene | 63 | 8 | 18 | 11 | 0 |
| DMAD | 1.5 | DBU | 0.1 | 0.22 | Toluene | 63 | 3 | 1 | 1 | 0 |
| DMAD | 1.5 | 1 | / | 0.22 | Toluene | 62 | 7 | 12 | 18 | 0 |
| DMAD | 1 | 2,6-lutidine | 1 | 0.11 | Toluene | 61 | 6 | 16 | 17 | 0 |
| Methyl propionate | 2 | / | / | 0.06 | Toluene | 61 | 4 | 10 | 25 | 0 |

dimethyl acetylenedicarboxylate (DMAD),
(2,2,6,6-tetramethylpiperidin-1-yl)oxyl (TEMPO),
diethyl fumarate (DEF),
diethyl acetylenedicarboxylate (DEAD),
2-mercaptobenzothiazole (MBT),
1,4-diazabicyclo[2.2.2]octane (DABCO),
diisopropyl azodicarboxylate (DIAD),
N,N-diisopropylethylamine (DIPEA),
trimethyl phosphite (TMP),

### Example 7: Preparation of 3-tert-butyldimethylsilyloxy-estra-1,3,5(10)-15-tetraene-17-(Compound 3)

3-tert-Butyldimethylsilyloxy-estra-1,3,5(10)-15-tetraene-17-ol (compound 3) was prepared as schematically illustrated in scheme 2.

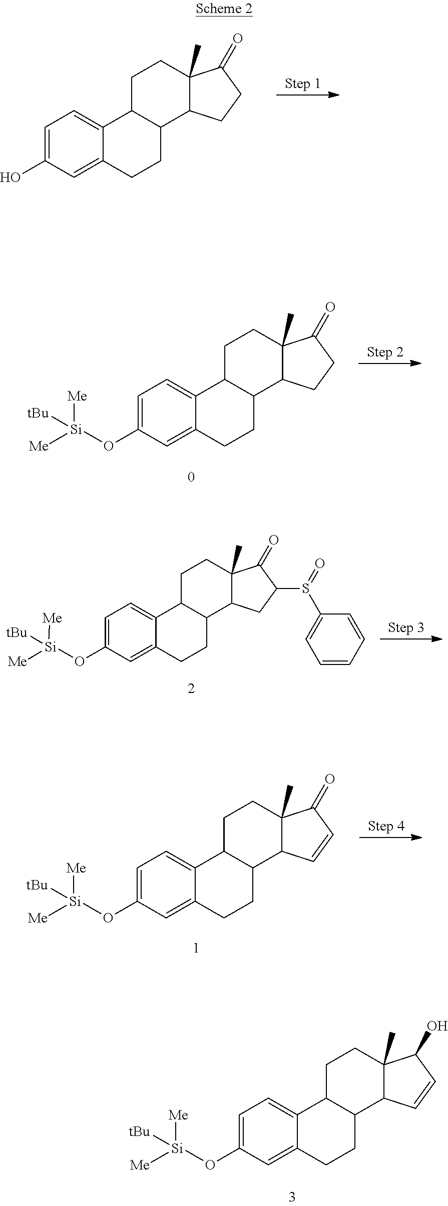 [SMILES: C[C@]12CCC3c4ccc(O)cc4CCC3C1CCC2=O ; CC(C)(C)[Si](C)(C)Oc1ccc2c(c1)CCC1C2CC[C@]2(C)C(=O)CCC12 ; CC(C)(C)[Si](C)(C)Oc1ccc2c(c1)CCC1C2CC[C@]2(C)C(=O)C(S(=O)c3ccccc3)CC12 ; CC(C)(C)[Si](C)(C)Oc1ccc2c(c1)CCC1C2CC[C@]2(C)C(=O)C=CC12 ; CC(C)(C)[Si](C)(C)Oc1ccc2c(c1)CCC1C2CC[C@@]2(C)C1C=C[C@@H]2O]

### Step 1: 3-tert-butyldimethylsilyloxy-estra-1,3,5(10)-triene-17-one

To a solution of 3-hydroxy-estra-1,3,5(10)-triene-17-one (100 g, 0.370 mole) in dichloromethane (500 mL) were added tert-butyldimethylsilyl-chloride (58.3 g, 0.388 mole) and imidazole (26.4 g, 0.388 mole). The mixture was stirred for 24 hours at room temperature. Water (300 mL) was added and the organic layer was washed with 200 mL of water. After concentration the product was crystallized from a mixture of ethanol/diisopropyl ether, collected by filtration and dried. The product is isolated as a white solid (145 g, 95%).

¹H-NMR (CDCl₃): δ 0.20 (s, 6H, (CH₃)₂—Si—), 0.90 (s, 3H, CH₃ at C-18), 1.00 (s, 9H, (CH₃)₃—C—Si), 1.20-2.60 (m, 13H), 2.75-2.95 (m, 2H), 5.65-5.75 (m, 1H), 6.58 (broad s, 1H, H4), 6.63 (dd, 1H, H2), 7.12 (d, 1H, H1).

### Step 2: 3-tert-butyldimethylsilyloxy-16-(phenylsulfinyl)-estra-1,3,5(10)-triene-17-one

3-tert-Butyldimethylsilyloxy-estra-1,3,5(10)-triene-17-one (250 mg, 0.650 mmol) was added to a suspension of sodium hydride (78.0 mg, 1.950 mmol, 60% in parafilm) in THF (7.1 mL) at room temperature under N₂ atmosphere. Methyl benzenesulfinate (152.3 mg, 0.975 mmol) was then added and the white mixture was stirred at room temperature for 60 min. The conversion of the starting material was controlled via TLC analysis [PE/EtOAc (8:2)]. The reaction was quenched with addition of a 5% aqueous solution of NaHCO₃ (15 mL) and extracted with toluene (2×15 mL). The organic layers were evaporated under reduced pressure and the crude product was purified by chromatography over silica gel (petroleum ether/EtOAc [8/2]) to afford the product as a white solid (139.5 mg, 42% yield).

¹H-NMR (CDCl₃): δ 0.17 (s, 6H, (CH₃)₂—Si—), 0.91 (s, 3H, CH₃ at C-18), 0.97 (s, 9H, (CH₃)₃—C—Si), 1.08-2.92 (m, 12H), 3.25-3.73 (m, 1H, H16), 6.53-6.58 (m, 1H, H4), 6.62 (d, 1H, H2), 7.04-7.14 (m, 1H, H1), 7.47-7.58 (m, 3H, PhS(O)—), 7.59-7.68 (m, 2H, PhS(O)—).

### Step 3: 3-tert-butyldimethylsilyloxy-estra-1,3,5(10)-15-tetraene-17-one

To a solution of 3-tert-butyldimethylsilyloxy-16-(phenylsulfinyl)-estra-1,3,5(10)-triene-17-one (2.89 g, 5.70 mmol) in toluene (24 mL) were successively added dimethyl acetylene-dicarboxylate (1.02 mL, 14.20 mmol) and triethylamine (0.08 mL, 0.57 mmol). The solution was injected in a tubing (Vᵢₙₜ=5 mL) heated at 210° C. with a flow rate of 0.833 mL/min. The feed was maintained liquid with a back-pressure regulator (BPR) of 34 bar (500 psi). The flow was then cooled to 0° C. and the crude reaction mixture is retrieved in a flask. The organic layer is washed with a 0.5M aqueous solution of NaOH (45 mL) and the organic layer was dried over MgSO₄. The volatiles were removed under reduced pressure and the crude product was purified by chromatography over silica gel (petroleum ether/EtOAc [8/2]) to afford the product as an off-white solid (2.43 g, 71% yield).

¹H-NMR (CDCl₃): δ 0.20 (s, 6H, (CH₃)₂—Si—), 1.00 (s, 9H, (CH₃)₃—C—Si—), 1.13 (s, 3H, CH₃ at C-18), 1.20-2.70 (m, 11H), 2.80-3.00 (m, 2H), 6.10 (dd, 1H, H15), 6.58 (broad s, 1 H, H4), 6.62 (dd, 1H, H2), 7.11 (d, 1H, H1), 7.63 (dd, 1H, H16).

### Step 4: 3-tert-butyldimethylsilyloxy-estra-1,3,5(10)-15-tetraene-17-ol

The material collected in step 3 was dissolved in THF (7.6 mL) and a solution of cerium chloride heptahydrate (3.19 g, 8.55 mmol) in methanol (8.0 mL) was added. The mixture was cooled to 0° C. and sodium borohydride (0.45 g, 11.97 mmol) was added portion wise keeping the temperature below 9° C. At the end of the addition the mixture was stirred for 1 h then quenched by addition of a 2N HCl solution (2.5 mL), extracted with ethyl acetate and washed with water. The organic layer was partly evaporated then diisopropyl ether was added. The precipitate was collected by filtration and dried. After crystallization form a mixture of ethanol/diisopropyl ether the title compound was isolated in 90% yield as an off-white solid. The yield over two steps is 63%.

¹H-NMR (CDCl₃): δ 0.20 (s, 6H, (CH₃)₂—Si—), 0.89 (s, 3H, CH₃ at C-18), 1.00 (s, 9H, (CH₃)₃—C—Si—), 1.20-2.40 (m, 10H), 2.75-2.95 (m, 2H), 4.40 (broad s, 1 H, H17), 5.65-5.75 (m, 1H), 5.95-6.10 (m, 1H), 6.57 (broad s, 1H, H4), 6.60 (dd, 1H, H2), 7.13 (d, 1H, H1).

Alternatively, the procedures of step 3 and 4 as described hereinabove may be combined with only a purification at the last step.

### Step 3+Step 4: 3-tert-butyldimethylsilyloxy-estra-1,3,5(10)-15-tetraene-17-ol

To a solution of 3-tert-butyldimethylsilyloxy-16-(phenylsulfinyl)-estra-1,3,5(10)-triene-17-one (10.0 g, 19.08 mmol) in toluene (84 mL) is successively added dimethyl acetylene-dicarboxylate (3.5 mL, 28.52 mmol) and triethylamine (0.3 mL, 1.90 mmol). The solution was injected in a tubing (Vᵢₙₜ=5 mL) heated at 210° C. with a flow rate of 0.833 mL/min. The feed was maintained liquid with a back-pressure regulator (BPR) of 34 bar (500 psi). The flow was then cooled to 0° C. and the crude reaction mixture was retrieved in a flask. The organic layer was washed with a 1M aqueous solution of NaOH (85 mL) and the organic layer was dried over MgSO₄. The volatiles were removed under reduced pressure and the residue was dissolved in THF (25 mL) and a solution of cerium chloride heptahydrate (10.66 g, 28.62 mmol) in methanol (29 mL) was added. The mixture was cooled to 0° C. and sodium borohydride (1.50 g, 40.07 mmol) was added portion wise keeping the temperature below 9° C. At this end of the addition the mixture was stirred for one hour then quenched by addition of a 2N HCl solution (8.0 mL), extracted with ethyl acetate and washed with water. The organic layer was partly evaporated then diisopropyl ether was added. The precipitate was collected by filtration and dried. After crystallization form a mixture of ethanol/diisopropyl ether the title compound was isolated as a beige solid (5.07 g, 69% over two steps).

¹H-NMR (CDCl₃): δ 0.20 (s, 6H, (CH₃)₂—Si—), 0.89 (s, 3H, CH₃ at C-18), 1.00 (s, 9H, (CH₃)₃—C—Si—), 1.20-2.40 (m, 10H), 2.75-2.95 (m, 2H), 4.40 (broad s, 1 H, H17), 5.65-5.75 (m, 1H), 5.95-6.10 (m, 1H), 6.57 (broad s, 1H, H4), 6.60 (dd, 1H, H2), 7.13 (d, 1H, H1).

### Example 8: Preparation of 3-tert-butyldimethylsilyloxy-estra-1,3,5(10)-15-tetraene-17-ol (Compound 3)

3-tert-butyldimethylsilyloxy-estra-1,3,5(10)-15-tetraene-17-ol (compound 3) was prepared as schematically illustrated in scheme 3.

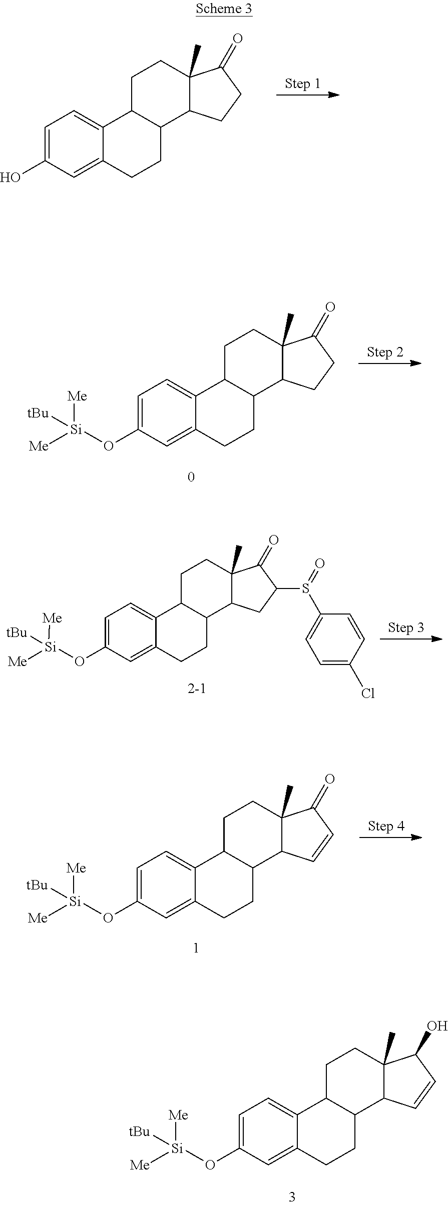 [SMILES: C[C@]12CCC3c4ccc(O)cc4CCC3C1CCC2=O ; CC(C)(C)[Si](C)(C)Oc1ccc2c(c1)CCC1C2CC[C@]2(C)C(=O)CCC12 ; CC(C)(C)[Si](C)(C)Oc1ccc2c(c1)CCC1C2CC[C@]2(C)C(=O)C(S(=O)c3ccc(Cl)cc3)CC12 ; CC(C)(C)[Si](C)(C)Oc1ccc2c(c1)CCC1C2CC[C@]2(C)C(=O)C=CC12 ; CC(C)(C)[Si](C)(C)Oc1ccc2c(c1)CCC1C2CC[C@@]2(C)C1C=C[C@@H]2O]

### Step 1: 3-tert-butyldimethylsilyloxy-estra-1,3,5(10)-triene-17-one

3-tert-Butyldimethylsilyloxy-estra-1,3,5(10)-triene-17-one was prepared as described in step 1 of Example 7.

### Step 2: 3-tert-butyldimethylsilyloxy-16-(4-chloro-phenylsulfinyl)-estra-1,3,5(10)-triene-17-one

3-tert-Butyldimethylsilyloxy-estra-1,3,5(10)-triene-17-one (250 mg, 0.650 mmol) was added to a suspension of sodium hydride (78.0 mg, 1.950 mmol, 60% in parafilm) in THF (7.1 mL) at room temperature under N₂ atmosphere. Methyl 4-chlorobenzenesulfinate (185.9 mg, 0.975 mmol) was then added and the white mixture was stirred at room temperature for 60 min. The conversion of the starting material was controlled via TLC analysis [PE/EtOAc (8:2)]. The reaction was quenched with addition of a 5% aqueous solution of NaHCO₃ (15 mL) and extracted with toluene (2×15 mL). The organic layers were evaporated under reduced pressure and the crude product was purified by chromatography over silica gel (petroleum ether/EtOAc [8/2]) to afford the product as a white solid.

¹H-NMR (CDCl₃): δ 0.17 (s, 6H, (CH₃)₂—Si—), 0.97 (s, 9H, (CH₃)₃—C—Si), 1.07-2.58 (m, 13H), 2.73-2.89 (m, 2H, H6), 3.23-3.63 (m, 1H, H16), 6.52-6.59 (m, 1H, H4), 6.59-6.65 (m, 1H, H2), 7.09 (d, 1H, H1), 7.49-7.55 (m, 2H), 7.59-7.68 (m, 2H).

### Step 3: 3-tert-butyldimethylsilyloxy-estra-1,3,5(10)-15-tetraene-17-one

To a solution of 3-tert-butyldimethylsilyloxy-16-(4-chlorophenylsulfinyl)-estra-1,3,5(10)-triene-17-one (1.19 g, 2.20 mmol) in toluene (9.6 mL) is successively added dimethyl acetylene-dicarboxylate (0.4 mL, 3.30 mmol) and triethylamine (0.03 mL, 0.22 mmol). The solution was injected in a tubing (Vᵢₙₜ=5 mL) heated at 210° C. with a flow rate of 0.833 mL/min. The feed was maintained liquid with a back-pressure regulator (BPR) of 500 psi (34 bar). The flow was then cooled to 0° C. and the crude reaction mixture was retrieved in a flask. The organic layer is washed with a 1M aqueous solution of NaOH (10 mL) and the organic layer is dried over MgSO₄. The volatiles were removed under reduced pressure to give a brown crude mixture containing an 91/6/2/1% area by HPLC of 3-tert-butyldimethylsilyloxy-estra-1,3,5(10)-15-tetraene-17-one/3-tert-butyldimethylsilyloxy-estra-1,3,5(10)-14-tetraene-17-one/3-tert-butyldimethylsilyloxy-estra-1,3,5(10)-triene-17-one/3-tert-butyldimethylsilyloxy-16-phenylthio-estra-1,3,5(10)-15-tetraene-17-one.

¹H-NMR (CDCl₃): δ 0.20 (s, 6H, (CH₃)₂—Si—), 1.00 (s, 9H, (CH₃)₃—C—Si—), 1.13 (s, 3H, CH₃ at C-18), 1.20-2.70 (m, 11H), 2.80-3.00 (m, 2H), 6.10 (dd, 1H, H15), 6.58 (broad s, 1 H, H4), 6.62 (dd, 1H, H2), 7.11 (d, 1H, H1), 7.63 (dd, 1H, H16).

### Step 4: 3-tert-butyldimethylsilyloxy-estra-1,3,5(10)-15-tetraene-17-ol

3-tert-Butyldimethylsilyloxy-estra-1,3,5(10)-tretraene-17-ol was prepared from 3-tert-butyldimethylsilyloxy-16-(phenylsulfinyl)-estra-1,3,5(10)-tetraene-17-one as described in step 4 of Example 7.

### Example 9: Preparation of 3-tert-butyldimethylsilyloxy-estra-1,3,5(10)-15-tetraene-17-ol (Compound 3)

3-tert-butyldimethylsilyloxy-estra-1,3,5(10)-15-tetraene-17-ol (compound 3) was prepared as schematically illustrated in scheme 4.

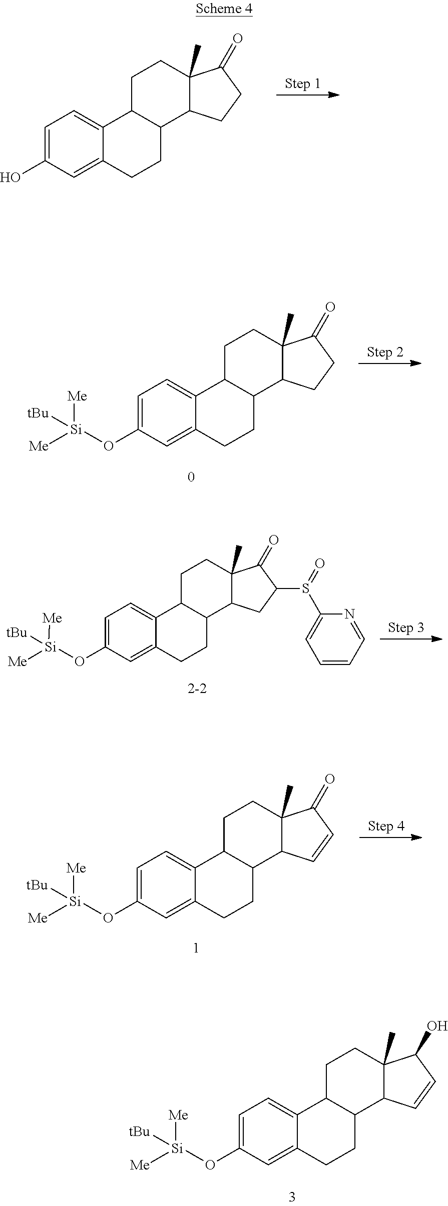 [SMILES: C[C@]12CCC3c4ccc(O)cc4CCC3C1CCC2=O ; CC(C)(C)[Si](C)(C)Oc1ccc2c(c1)CCC1C2CC[C@]2(C)C(=O)CCC12 ; CC(C)(C)[Si](C)(C)Oc1ccc2c(c1)CCC1C2CC[C@]2(C)C(=O)C(S(=O)c3ccccn3)CC12 ; CC(C)(C)[Si](C)(C)Oc1ccc2c(c1)CCC1C2CC[C@]2(C)C(=O)C=CC12 ; CC(C)(C)[Si](C)(C)Oc1ccc2c(c1)CCC1C2CC[C@@]2(C)C1C=C[C@@H]2O]

### Step 1: 3-tert-butyldimethylsilyloxy-estra-1,3,5(10)-triene-17-one

3-tert-Butyldimethylsilyloxy-estra-1,3,5(10)-triene-17-one was prepared as described in step 1 of Example 7.

### Step 2: 3-tert-butyldimethylsilyloxy-16-(2-pyridinesulfinyl)-estra-1,3,5(10)-triene-17-one

3-tert-Butyldimethylsilyloxy-estra-1,3,5(10)-triene-17-one (250 mg, 0.650 mmol) was added to a suspension of sodium hydride (78.0 mg, 1.950 mmol, 60% in parafilm) in THF (7.1 mL) at room temperature under N₂ atmosphere. Methyl 2-pyridinesulfinate (153.3 mg, 0.975 mmol) was then added and the white mixture was stirred at room temperature for 60 min. The conversion of the starting material was controlled via TLC analysis [PE/EtOAc (8:2)]. The reaction was quenched with addition of a 5% aqueous solution of NaHCO₃ (15 mL) and extracted with toluene (2×15 mL). The organic layers were evaporated under reduced pressure and the crude product was purified by chromatography over silica gel (petroleum ether/EtOAc [8/2]) to afford the product as a white solid.

### Step 3: 3-tert-butyldimethylsilyloxy-estra-1,3,5(10)-15-tetraene-17-one

To a solution of 3-tert-butyldimethylsilyloxy-16-(2-pyridinesulfinyl)-estra-1,3,5(10)-triene-17-one (1.12 g, 2.20 mmol) in toluene (9.6 mL) is successively added dimethyl acetylene-dicarboxylate (0.4 mL, 3.30 mmol) and triethylamine (0.03 mL, 0.22 mmol). The solution was injected in a tubing (Vᵢₙₜ=5 mL) heated at 210° C. with a flow rate of 0.833 mL/min. The feed was maintained liquid with a back-pressure regulator (BPR) of 34 bar (500 psi). The flow was then cooled to 0° C. and the crude reaction mixture was retrieved in a flask. The organic layer is washed with a 1M aqueous solution of NaOH (10 mL) and the organic layer was dried over MgSO₄. The volatiles were removed under reduced pressure to give a brown crude mixture containing an 86/13/1% area by HPLC of 3-tert-butyldimethylsilyloxy-estra-1,3,5(10)-15-tetraene-17-one/3-tert-butyldimethylsilyloxy-16-(2-pyridinesulfinyl)-estra-1,3,5(10)-triene-17-one/3-tert-butyldimethylsilyloxy-estra-1,3,5(10)-triene-17-one.

¹H-NMR (CDCl₃): δ 0.20 (s, 6H, (CH₃)₂—Si—), 1.00 (s, 9H, (CH₃)₃—C—Si—), 1.13 (s, 3H, CH₃ at C-18), 1.20-2.70 (m, 11H), 2.80-3.00 (m, 2H), 6.10 (dd, 1H, H15), 6.58 (broad s, 1 H, H4), 6.62 (dd, 1H, H2), 7.11 (d, 1H, H1), 7.63 (dd, 1H, H16).

### Step 4: 3-tert-butyldimethylsilyloxy-estra-1,3,5(10)-15-tetraene-17-ol

3-tert-Butyldimethylsilyloxy-estra-1,3,5(10)-tretraene-17-ol was prepared from 3-tert-butyldimethylsilyloxy-16-(phenylsulfinyl)-estra-1,3,5(10)-tetraene-17-one as described in step 4 of Example 7.

### Example 10: Preparation of 3-tert-butyldimethylsilyloxy-estra-1,3,5(10)-15-tetraene-17-ol (Compound 3)

3-tert-butyldimethylsilyloxy-estra-1,3,5(10)-15-tetraene-17-ol (compound 3) was prepared as schematically illustrated in Scheme 5.

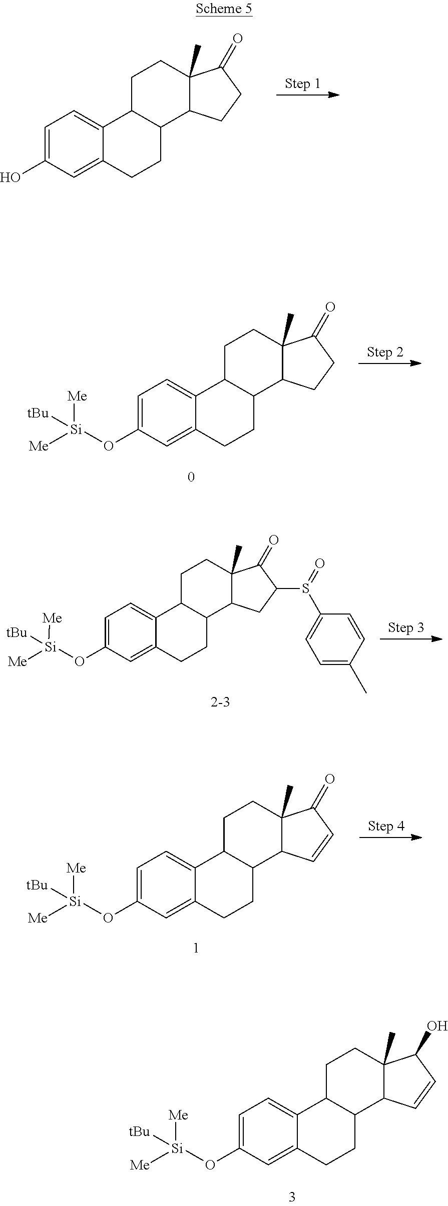 [SMILES: C[C@]12CCC3c4ccc(O)cc4CCC3C1CCC2=O ; CC(C)(C)[Si](C)(C)Oc1ccc2c(c1)CCC1C2CC[C@]2(C)C(=O)CCC12 ; Cc1ccc(S(=O)C2CC3C4CCc5cc(O[Si](C)(C)C(C)(C)C)ccc5C4CC[C@]3(C)C2=O)cc1 ; CC(C)(C)[Si](C)(C)Oc1ccc2c(c1)CCC1C2CC[C@]2(C)C(=O)C=CC12 ; CC(C)(C)[Si](C)(C)Oc1ccc2c(c1)CCC1C2CC[C@@]2(C)C1C=C[C@@H]2O]

### Step 1: 3-tert-butyldimethylsilyloxy-estra-1,3,5(10)-triene-17-one

3-tert-Butyldimethylsilyloxy-estra-1,3,5(10)-triene-17-one was prepared as described in step 1 of Example 7.

### Step 2: 3-tert-butyldimethylsilyloxy-16-(p-toluenesulfinyl)-estra-1,3,5(10)-triene-17-one

3-tert-Butyldimethylsilyloxy-estra-1,3,5(10)-triene-17-one (250 mg, 0.650 mmol) was added to a suspension of sodium hydride (78.0 mg, 1.950 mmol, 60% in parafilm) in THF (7.1 mL) at room temperature under N₂ atmosphere. Methyl p-toluenesulfinate (166.0 mg, 0.975 mmol) was then added and the white mixture was stirred at room temperature for 60 min. The conversion of the starting material was controlled via TLC analysis [PE/EtOAc (8:2)]. The reaction was quenched with addition of a 5% aqueous solution of NaHCO₃ (15 mL) and extracted with toluene (2×15 mL). The organic layers were evaporated under reduced pressure and the crude product was purified by chromatography over silica gel (petroleum ether/EtOAc [8/2]) to afford the product as a white solid.

¹H-NMR (CDCl₃): δ 0.17 (s, 6H, (CH₃)₂—Si—), 0.97 (s, 9H, (CH₃)₃—C—Si), 1.09-2.56 (m, 13H), 2.73-2.89 (m, 2H, H6), 3.24-3.69 (m, 1H, H16), 6.52-6.59 (m, 1H, H4), 6.59-6.64 (m, 1H, H2), 7.09 (d, 1H, H1), 7.29-7.37 (m, 2H), 7.47-7.56 (m, 2H).

### Step 3: 3-tert-butyldimethylsilyloxy-estra-1,3,5(10)-15-tetraene-17-one

To a solution of 3-tert-butyldimethylsilyloxy-16-(p-toluenesulfinyl)-estra-1,3,5(10)-triene-17-one (1.15 g, 2.20 mmol) in toluene (9.6 mL) was successively added dimethyl acetylene-dicarboxylate (0.4 mL, 3.30 mmol) and triethylamine (0.03 mL, 0.22 mmol). The solution was injected in a tubing (Vᵢₙₜ=5 mL) heated at 210° C. with a flow rate of 0.833 mL/min. The feed is maintained liquid with a back-pressure regulator (BPR) of 34 bar (500 psi). The flow was then cooled to 0° C. and the crude reaction mixture was retrieved in a flask. The organic layer was washed with a 1M aqueous solution of NaOH (10 mL) and the organic layer was dried over MgSO₄. The volatiles were removed under reduced pressure to give a brown crude mixture containing a 59/41% area by HPLC of 3-tert-butyldimethylsilyloxy-estra-1,3,5(10)-15-tetraene-17-one/3-tert-butyldimethylsilyloxy-16-(p-toluenesulfinyl)-estra-1,3,5(10)-triene-17-one.

¹H-NMR (CDCl₃): δ 0.20 (s, 6H, (CH₃)₂—Si—), 1.00 (s, 9H, (CH₃)₃—C—Si—), 1.13 (s, 3H, CH₃ at C-18), 1.20-2.70 (m, 11H), 2.80-3.00 (m, 2H), 6.10 (dd, 1H, H15), 6.58 (broad s, 1 H, H4), 6.62 (dd, 1H, H2), 7.11 (d, 1H, H1), 7.63 (dd, 1H, H16).

### Step 4: 3-tert-butyldimethylsilyloxy-estra-1,3,5(10)-15-tetraene-17-ol

3-tert-Butyldimethylsilyloxy-estra-1,3,5(10)-tretraene-17-ol was prepared from 3-tert-butyldimethylsilyloxy-16-(phenylsulfinyl)-estra-1,3,5(10)-tetraene-17-one as described in step 4 of Example 7.

### Example 11: Preparation of 3-tert-butyldimethylsilyloxy-estra-1,3,5(10)-15-tetraene-17-ol (Compound 3, See Scheme 2) Using a Two Pumps Setup

### Step 1: 3-tert-butyldimethylsilyloxy-estra-1,3,5(10)-triene-17-one

3-tert-Butyldimethylsilyloxy-estra-1,3,5(10)-triene-17-one was prepared as described in step 1 of Example 7.

### Step 2: 3-tert-butyldimethylsilyloxy-16-(phenylsulfinyl)-estra-1,3,5(10)-triene-17-one

3-tert-butyldimethylsilyloxy-16-(phenylsulfinyl)-estra-1,3,5(10)-triene-17-one was prepared as described in step 2 of Example 7.

### Step 3: 3-tert-butyldimethylsilyloxy-estra-1,3,5(10)-15-tetraene-17-one

A solution of 3-tert-butyldimethylsilyloxy-16-(phenylsulfinyl)-estra-1,3,5(10)-triene-17-one (4.00 g, 7.19 mmol) and Et₃N (0.1 mL, 0.72 mmol) in toluene (31.2 mL) was pumped with “pump A” (see Scheme 6) with a flow rate of 2.440 mL/min. A solution of DMAD (1.3 mL, 10.79 mmol) in toluene (2.7 mL) was pumped with “pump B” with a flow rate of 0.220 mL/min. Flow A and blow B are mixed in a “T mixer” and the combined flow were injected in a tubing (Vᵢₙₜ=16 mL) heated at 210° C. with a global flow rate of 2.660 mL/min. The feed was maintained liquid with a back-pressure regulator of 34 bar (500 psi). The flow was then cooled to 0° C. and the crude reaction mixture was retrieved in a flask. The organic layer was washed with a 1M aqueous solution of NaOH (30 mL) and the organic layer was dried over MgSO₄. The volatiles were removed under reduced pressure to give a brown crude mixture containing an 80/13/2/5% area by HPLC of 3-tert-butyldimethylsilyloxy-estra-1,3,5(10)-15-tetraene-17-one/3-tert-butyldimethylsilyloxy-estra-1,3,5(10)-triene-17-one/3-tert-butyldimethylsilyloxy-estra-1,3,5(10)-14-tetraene-17-one/3-tert-butyldimethylsilyloxy-16-phenylthio-estra-1,3,5(10)-15-tetraene-17-one.

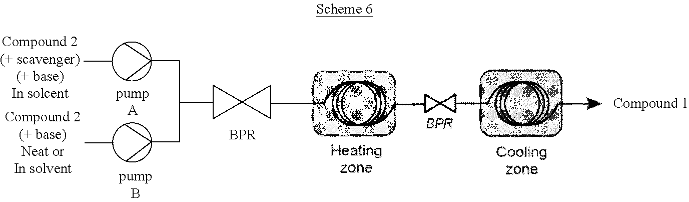

¹H-NMR (CDCl₃): δ 0.20 (s, 6H, (CH₃)₂—Si—), 1.00 (s, 9H, (CH₃)₃—C—Si—), 1.13 (s, 3H, CH₃ at C-18), 1.20-2.70 (m, 11H), 2.80-3.00 (m, 2H), 6.10 (dd, 1H, H15), 6.58 (broad s, 1 H, H4), 6.62 (dd, 1H, H2), 7.11 (d, 1H, H1), 7.63 (dd, 1H, H16).

### Step 4: 3-tert-butyldimethylsilyloxy-estra-1,3,5(10)-15-tetraene-17-ol

3-tert-Butyldimethylsilyloxy-estra-1,3,5(10)-15-tetraene-17-ol was prepared as described in step 4 of Example 7.

### Example 12: Preparation of 3-tert-butyldiphenylsilyloxy-estra-1,3,5(10)-15-tetraene-17-ol

### Step 1: 3-tert-butyldiphenylsilyloxy-estra-1,3,5(10)-triene-17-one

To a solution of 3-hydroxy-estra-1,3,5(10)-triene-17-one (10.00 g, 36.98 mmol) and imidazole (5.04 g, 73.97 mmol) in dimethylformamide (74 ml) is added tert-butyl (chloro)diphenylsilane (11.18 g, 40.68 mmol). The mixture is stirred for 24 hours at room temperature. The reaction mixture is diluted with dichloromethane (200 mL) and water (200 mL) is added. The organic layer is washed respectively with an aqueous 10% NH₄Cl solution (100 mL), an aqueous 10% NaHCO₃ solution (100 mL) and brine (100 mL). Evaporation of the volatiles afford 3-tert-butyldiphenylsilyloxy-estra-1,3,5(10)-triene-17-one as an off-white solid (13.33 g, 84%).

¹H-NMR (CDCl₃): δ 0.89 (3H, s, CH₃ at C-18), 1.08 (9H, s, (CH₃)₃—Si—), 1.34-1.62 (6H, m), 1.86-1.97 (2H, m), 1.97-2.04 (1H, m), 2.06-2.38 (3H, m), 2.48 (1H, dd), 2.65-2.82 (2H, m), 6.49 (1H, dd), 6.58 (1H, d), 6.96 (1H, d), 7.34-7.44 (6H, m), 7.70-7.75 (4H, m).

### Step 2: 3-tert-butyldiphenylsilyloxy-16-(phenylsulfinyl)-estra-1,3,5(10)-triene-17-one

3-tert-Butyldiphenylsilyloxy-estra-1,3,5(10)-triene-17-one (6.34 g, 12.47 mmol) is added to a suspension of sodium hydride (1.50 g, 37.40 mmol, 60% in parafilm) in THF (55 mL) at room temperature under N₂ atmosphere. Methyl benzenesulfinate (2.92 g, 18.70 mmol) is then added to the reaction mixture and the white mixture is stirred at room temperature for 1 h0 min. The conversion of the starting material is controlled via TLC analysis [PE/EtOAc (8:2)]. The reaction is quenched with addition of an aqueous 5% solution of NaHCO₃ (215 mL) and extracted with toluene (2×215 mL). The organic layers are evaporated under reduced pressure and the crude product is purified by chromatography over silica gel to afford 3-tert-butyldiphenylsilyloxy-16-(phenylsulfinyl)-estra-1,3,5(10)-triene-17-one as a white solid (4.67 g, 59%).

¹H-NMR (CDCl₃): δ 0.96 (3H, s, CH₃ at C-18), 1.09 (9H, s, (CH₃)₃—Si—), 1.16-2.38 (14H, m), 2.42-2.54 (1H, m), 2.65-2.76 (2H, m), 3.24-3.72 (1H, m), 6.49 (1H, d), 6.52-6.58 (1H, m), 6.95 (1H, d), 7.31-7.48 (6H, m), 7.47-7.59 (3H, m), 7.59-7.67 (2H, m), 7.67-7.78 (4H, m).

### Step 3: 3-tert-butyldiphenylsilyloxy-estra-1,3,5(10)-15-tetraene-17-one

To a solution of 3-tert-butyldiphenylsilyloxy-16-(phenylsulfinyl)-estra-1,3,5(10)-triene-17-one (2.59 g, 4.10 mmol) in toluene (17.8 mL) was successively added dimethyl acetylene-dicarboxylate (0.8 mL, 6.16 mmol) and triethylamine (0.06 mL, 0.41 mmol). The solution is injected in a tubing (Vᵢₙₜ=5 mL) heated at 210° C. with a flow rate of 0.833 mL/min. The feed was maintained liquid with a back-pressure regulator of 34 bar (500 psi). The flow is then cooled to 0° C. and the crude reaction mixture was retrieved in a flask. The organic layer is washed with a 1M aqueous solution of NaOH (20 mL) and the organic layer is dried over MgSO₄. The volatiles are removed under reduced pressure and the crude product is purified by chromatography over silica gel (petroleum ether/EtOAc [8/2]) to afford the product as an off-white solid (1.78 g, 86%).

¹H-NMR (CDCl₃): δ 1.09 (9H, s, (CH₃)₃—Si—), 1.38-1.87 (7H, m), 1.97 (1H, d), 2.06-2.16 (1H, m), 2.21-2.39 (2H, m), 2.46 (1H, d), 2.71-2.84 (2H, m), 6.07 (1H, dd), 6.51 (1H, dd), 6.56 (1H, dd), 6.96 (1H, d), 7.34-7.46 (6H, m), 7.60 (1H, d), 7.68-7.78 (4H, m).

### Step 4: 3-tert-butyldiphenylsilyloxy-estra-1,3,5(10)-15-tetraene-17-ol

3-tert-Butyldiphenylsilyloxy-estra-1,3,5(10)-15-tetraene-17-ol is prepared as described in step 4 of Example 7.

The product is an off-white solid (362 mg, 68%).

¹H-NMR (CDCl₃): δ 0.87 (3H, s), 1.13 (9H, s, (CH₃)₃—Si—), 1.52-1.71 (3H, m), 1.84 (1H, bs), 1.95-2.10 (3H, m), 2.17-2.31 (2H, m), 2.66-2.85 (2H, m), 4.41 (1H, s), 5.71-5.76 (1H, m), 6.03 (1H, d), 6.54 (1H dd), 6.60 (1H, d), 7.00 (1H, d), 7.36-7.48 (6H, m), 7.73-7.82 (4H, m).

### Example 13: Preparation of 3-benzyloxy-estra-1,3,5(10)-15-tetraene-17-ol

### Step 1: 3-benzyloxy-estra-1,3,5(10)-triene-17-one

To a solution of 3-hydroxy-estra-1,3,5(10)-triene-17-one (10.00 g, 36.97 mmol) at room temperature in acetonitrile (148 mL) is added successively potassium carbonate (12.78 g, 92.47 mmol) and benzyl bromide (4.8 mL, 40.69 mmol). The mixture is stirred at reflux for 24 hours. The reaction mixture was cooled down and quenched with an aqueous 10% NH₄Cl solution (150 mL). The organic layer is washed with an aqueous 10% NaHCO₃solution (150 mL), brine (150 mL) and the volatiles are removed under reduced pressure to afford 3-benzyloxy-estra-1,3,5(10)-triene-17-one as a white solid (4.36 g, 33%).

¹H-NMR (CDCl₃): δ 0.91 (3H, s, CH₃ at C-18), 1.38-1.72 (6H, m), 1.91-2.21 (3H, m), 2.21-2.32 (1H, m), 2.36-2.46 (1H, m), 2.46-2.57 (1H, m), 2.70 (1H, dd), 2.85-2.94 (2H, m), 5.04 (2H, s), 6.74 (1H, s), 6.80 (1H, dd), 7.21 (1H, d), 7.31-7.45 (5H, m).

### Step 2: 3-benzyloxy-16-(phenylsulfinyl)-estra-1,3,5(10)-triene-17-one

To a suspension of potassium tert-butoxide (1.01 g, 9.00 mmol) in tetrahydrofuran (13.3 mL) at room temperature under N₂ atmosphere 3-benzyloxy-estra-1,3,5(10)-triene-17-one (1.08 g, 3.00 mmol) is added. The mixture is stirred at room temperature for 1 h and then methyl benzenesulfinate (0.70 g, 4.50 mmol) is added in one portion. After stirring for 72 hours the reaction mixture is poured into an aqueous 1M solution of HCl (25 mL). The organic layer is washed with a 5% aqueous solution of NaHCO₃ (25 mL) and brine (25 mL). The volatiles are removed under reduced pressure to afford 3-benzyloxy-16-(phenylsulfinyl)-estra-1,3,5(10)-triene-17-one as an off-white solid (1.23 g, 85%).

¹H-NMR (CDCl₃): δ 0.99 (3H, d, CH₃ at C-18), 1.46-2.55 (11H, m), 2.76-2.96 (2H, m), 3.27-3.72 (2H, m), 5.03 (2H, s), 6.70-6.75 (1H, m), 6.78 (1H, d), 7.19 (1H, d), 7.28-7.48 (5H, m), 7.48-7.60 (3H, m), 7.60-7.69 (2H, m).

### Step 3: 3-benzyloxy-estra-1,3,5(10)-15-tetraene-17-one

To a solution of 3-benzyloxy-16-(phenylsulfinyl)-estra-1,3,5(10)-triene-17-one (626 mg, 1.29 mmol) in toluene (10.0 mL) is successively added dimethyl acetylene-dicarboxylate (0.4 mL, 3.30 mmol) and triethylamine (0.03 mL, 0.22 mmol). The solution is injected in a tubing (Vᵢₙₜ=5 mL) heated at 210° C. with a flow rate of 0.833 mL/min. The feed is maintained liquid with a back-pressure regulator of 34 bar (500 psi). The flow is then cooled to 0° C. and the crude reaction mixture is retrieved in a flask. The organic layer is washed with a 1M aqueous solution of NaOH (10 mL) and the organic layer is dried over MgSO₄. The volatiles are removed under reduced pressure and the crude product is purified by chromatography over silica gel (petroleum ether/EtOAc [8/2]) to afford the product as an off-white solid (327 mg, 71%).

¹H-NMR (CDCl₃): δ 1.11 (3H, s, CH₃ at C-18), 1.53-1.84 (4H, m), 1.96-2.08 (1H, m), 2.15-2.25 (1H, m), 2.31-2.40 (1H, m), 2.40-2.48 (1H, m), 2.53 (1H, d), 2.89-3.03 (2H, m), 5.04 (2H, s), 6.03-6.15 (1H, dd), 6.74 (1H, bs), 6.81 (1H, d), 7.21 (1H, d), 7.28-7.47 (5H, m), 7.62 (1H, d).

### Step 4: 3-benzyloxy-estra-1,3,5(10)-15-tetraene-17-ol

3-benzyloxy-estra-1,3,5(10)-15-tetraene-17-ol is prepared as described in step 4 of Example 7.

The product is an off-white solid (229 mg, 70%).

¹H-NMR (CDCl₃): δ 0.86 (3H, s, CH₃ at C-18), 1.37-1.52 (1H, m), 1.56-1.73 (4H, m), 2.00-2.12 (2H, m), 2.22-2.42 (2H, m), 2.83-3.00 (2H, m), 4.42 (1H, s), 5.05 (2H, s), 5.66-5.78 (1H, m), 6.03 (1H, d), 6.73 (1H, dd), 6.82 (1H, dd), 7.20 (1H, d), 7.30-7.46 (4H, m).

### Example 14: Preparation of 3-triisopropylsilyloxy-estra-1,3,5(10)-15-tetraene-17-ol

### Step 1: 3-triisopropylsilyloxy-estra-1,3,5(10)-triene-17-one

To a solution of 3-hydroxy-estra-1,3,5(10)-triene-17-one (10.00 g, 36.99 mmol) and imidazole (7.55 g, 110.97 mmol) in dimethylformamide (148 ml) is added triisopropylsilyl chloride (8.56 g, 44.39 mmol). The solution is stirred for 17 hours at room temperature. The reaction mixture is diluted with diethyl ether (200 mL) and water (150 mL) is added. The aqueous layer is extracted twice with diethyl ether (2×100 mL). The combined organic layers were washed respectively with brine (150 mL) and dried over MgSO₄. Filtration and evaporation of the volatiles afford 3-triisopropylsilyloxy-estra-1,3,5(10)-triene-17-one as a white solid (13.30 g, 84%).

¹H-NMR (CDCl₃): δ 0.91 (3H, s, CH₃ at C-18), 1.05-1.11 (21H, m), 1.18-1.32 (3H, m), 1.36-1.69 (4H, m), 1.90-2.28 (4H, m), 2.34-2.41 (1H, m), 2.50 (1H, dd), 2.82-2.88 (2H, m), 6.61 (1H, d), 6.66 (1H, dd), 7.10 (1H, d).

### Step 2: 3-triisopropylsilyloxy-16-(phenylsulfinyl)-estra-1,3,5(10)-triene-17-one

3-triisopropylsilyloxy-estra-1,3,5(10)-triene-17-one (5.00 g, 11.72 mmol) is added to a suspension of sodium hydride (1.18 g, 35.15 mmol, 60% in parafilm) in THF (44 mL) at room temperature under N₂ atmosphere. Methyl benzenesulfinate (2.75 g, 17.58 mmol) is then added to the reaction mixture and the white mixture is stirred at room temperature for 1 h0 min. The conversion of the starting material is controlled via TLC analysis [PE/EtOAc (8:2)]. The reaction is quenched with addition of an aqueous 5% solution of NaHCO₃ (50 mL) and extracted with toluene (2×50 mL). The organic layers are evaporated under reduced pressure and the crude product is purified by chromatography over silica gel (petroleum ether/EtOAc [7/3]) to afford 3-triisopropylsilyloxy-16-(phenylsulfinyl)-estra-1,3,5(10)-triene-17-one as a white solid (4.76 g, 74%).

¹H-NMR (CDCl₃): δ 0.99 (3H, s, CH₃ at C-18), 1.06-1.14 (21H, m), 1.18-1.29 (4H, m), 1.41-2.55 (7H, m), 2.72-2.91 (2H, m), 3.27-3.74 (1H, m), 6.59 (1H, d), 6.64 (1H, dd), 7.09 (1H, d), 7.48-7.59 (3H, m), 7.65 (2H, t).

### Step 3: 3-triisopropylsilyloxy-estra-1,3,5(10)-15-tetraene-17-one

To a solution of 3-triisopropylsilyloxy-16-(phenylsulfinyl)-estra-1,3,5(10)-triene-17-one (1.21 g, 2.20 mmol) in toluene (9.6 mL) was successively added dimethyl acetylene-dicarboxylate (0.4 mL, 3.30 mmol) and triethylamine (0.03 mL, 0.22 mmol). The solution is injected in a tubing (Vᵢₙₜ=5 mL) heated at 210° C. with a flow rate of 0.833 mL/min. The feed was maintained liquid with a back-pressure regulator of 34 bar (500 psi). The flow is then cooled to 0° C. and the crude reaction mixture was retrieved in a flask. The organic layer is washed with a 1M aqueous solution of NaOH (10 mL) and the organic layer is dried over MgSO₄. The volatiles are removed under reduced pressure and the crude product is purified by chromatography over silica gel (petroleum ether/EtOAc [8/2]) to afford the product as an off-white solid (414 mg, 44%).

¹H-NMR (CDCl₃): δ 1.06-1.15 (21H, m), 1.19-1.30 (4H, m), 1.47-1.89 (5H, m), 1.98-2.06 (1H, m), 2.13-2.22 (1H, m), 2.28-2.38 (1H, m), 2.38-2.46 (1H, m), 2.46-2.56 (1H, m), 2.80-3.00 (1H, m), 6.09 (1H, dd), 6.63 (1H, d), 6.68 (1H, dd), 7.11 (1H, d), 7.62 (1H, d).

### Step 4: 3-triisopropylsilyloxy-estra-1,3,5(10)-15-tetraene-17-ol

3-triisopropylsilyloxy-estra-1,3,5(10)-15-tetraene-17-ol was prepared as described in step 4 of Example 7.

The product is an off-white solid (310 mg, 75%).

¹H-NMR (CDCl₃): δ 0.86 (3H, s, CH₃ at C-18), 1.04-1.30 (21H, m), 1.35-1.51 (1H, m), 1.57-1.73 (3H, m), 1.97-2.03 (2H, m), 2.05-2.10 (2H, m), 2.19-2.38 (2H, m), 2.77-2.94 (2H, m), 4.33-4.50 (1H, m), 5.71 (1H, m), 6.02 (1H, d), 6.60 (1H, d), 6.66 (1H, dd), 7.08 (1H, d).

### Example 15: Preparation of estra-1,3,5(10),-triene 3,15α,16α,17β-tetrol of Formula (E) Via Compound (B) Using Tert-Butyldimethylsilyl Group as Protecting Group for R¹ and R^{2a} According to an Embodiment of the Invention

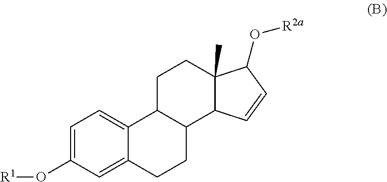

### Step 1: estra-1,3,5(10), 15-tetraene-3,17β-diol bis(tert-butyldimethylsilyl) ether

The starting material 3-tert-butyldimethylsiloxy-estra-1,3,5(10)-15-tetraene-17β-ol can be prepared as described in Examples 7 to 11. To a solution of 3-tert-butyldimethylsiloxy-estra-1,3,5(10)-15-tetraene-17β-ol (10.00 g, 25.0 mmol) in 100 ml of dimethylformamide were added imidazole (4.40 g, 65.0 mmol) and tert-butyldimethylsilyl chloride (14.70 g, 97.5 mmol) and allowed to stand at room temperature for 6 hours. The resulting solution was diluted with ethyl acetate, washed with water and evaporated. The residue was crystallized from methanol to afford (10.00 g, 80%) of estra-1,3,5(10), 15-tetraene-3,17β-diol bis(dimethyl-tert-butylsilyl) ether.

¹H-NMR (CDCl₃): δ 0.08 (s, 6H, 17-OSi(CH₃)₂, 0.18 (s, 6H, 3-OSi(CH₃)₂, 0.81 (s, 3H, 18-CH₃), 0.91 (s, 9H, 17-OSi-t-Bu), 0.97 (s, 9H, 3-OSi-t-Bu), 4.33 (broad s, 1H, H17), 5.60 (m, 1H,H-15), 5.95 (d, 1H, H16), 6.45-6.75 (m, 2H, H2 and H4), 7.12 (d, 1H, H1). mp: 89-91° C.

### Step 2: estra-1,3,5(10),-triene 3,15α,16α,17β-tetrol

To a stirred solution of estra-1,3,5(10), 15-tetraene-3,17β-diol bis(dimethyl-tert-butylsilyl) ether (10.00 g, 20.0 mmol) and formic acid (2.3 mL, 60.0 mmol) in acetone (100 mL) at 0° C. was added gradually a solution of potassium permanganate (3.15 g, 20.0 mmol) in water (20 mL) and acetone (100 mL). After completion of the reaction, the reaction was quenched with a 10% aqueous solution of KHSO₃. Acetone was partially removed and extracted with ethyl acetate, and washed with water. Ethyl acetate was concentrated under reduced pressure and diluted with heptane. The precipitate was collected by filtration and dissolved in acetone (100 mL). To the solution 5N hydrochloric acid (20 mL) was added. After completion of the reaction the resulting solution was diluted with water. The solid was collected by filtration, washed with heptane and crystallized from a mixture of methanol and water to afford the title compound.

### Example 16: Preparation of estra-1,3,5(10),-triene 3,15α,16α,17β-tetrol of Formula (E) Via Compound (B) Using Tert-Butyldimethylsilyl Group as Protecting Group for R¹ and pivaloyl for R^{2a} According to an Embodiment of the Invention

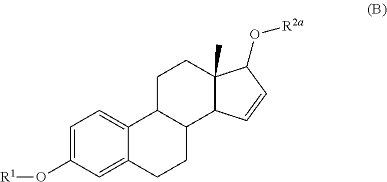

The starting material 3-tert-butyldimethylsiloxy-estra-1,3,5(10)-15-tetraene-17β-ol can be prepared as described in Examples 7 to 11. To a solution of 3-tert-butyldimethylsilyloxy-estra-1,3,5(10)-15-tetraene-17-ol (30.00 g, 78.0 mmol) in 300 mL of dichloromethane and 11 mL of triethylamine was added drop wise pivaloyl chloride (10.36 g, 86.0 mmol) in 50 mL of methylene chloride at 0° C. At the end of the addition the solution was stirred at room temperature for 1 hour. Water was added and the organic layer was washed two time with 100 ml of water. Heptane was added and the product was collected by filtration and used in the next step without any other purification.

3-tert-Butyl-dimethylsilyloxy-estra-1,3,5(10)-15-tetraene-17β-pivaloate was converted to its 15α,16α derivative following the procedure described in Example 15 step 2.

Then this 3-tert-butyldimethylsilyloxy-estra-1,3,5(10)-15α,16α-diol-triene-17β-pivaloate (10.00 g, 20.0 mmol) and K₂CO₃ (2.76 g, 20.0 mmol) was suspended in methanol (200 mL) and stirred for 4 hours at room temperature. Water (300 mL) was added and the mixture was neutralized with 0.1 N HCl. The product was collected by filtration and dried to afford 7.50 g (90% yield) of 3-tert-butyldimethylsilyloxy-estra-1,3,5(10)-triene-15α,16α,17β-triol.

Deprotection in acidic medium of the silyl protecting group was performed using the same conditions as described in example 15 step 2, and allowed this compound to be converted to estra-1,3,5(10),-triene 3,15α,16α,17β-tetrol in 90% yield.

### Example 17: Computational Calculation of the Effects of Different Substituents on a Compound of Formula (II) According to the Invention, and More Specifically on the Activation Energy in a Desulfinylation Process According to the Invention

An in silico approach was used to calculate transition states (TSs) and compute activation energies in the desulfinylation reaction of a compound of formula (W) (see Table 7 for the definition of R). Constant rates were determined accordingly to assess the reaction time needed to reach a specific conversion at a given temperature using the appropriate rate law. In this case, thermolysis phenomena rely on first order kinetics. This method enabled to evaluate the modulation effects of various substituents (R) on the activation barrier, and thus their effect on reaction time.

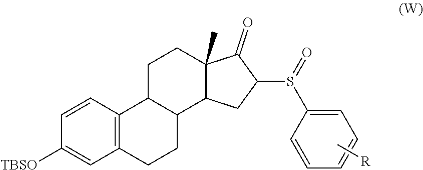

The unsubstituted derivative (compound Y, R═H,) was used as a reference compound to calibrate the model.

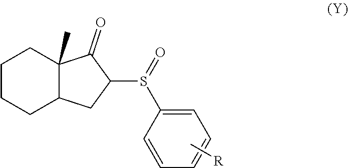

Calculations were performed using a commercial software (Gaussian 16 package) employing implicit solvation (SMD, solvent=toluene). Density Functional Theory (DFT)-based kinetics data about thermolysis of different R substituents of compound (W) are depicted in Table 7.

TABLE 7

| | | ΔΔGT^{‡} | t_{99% conv.} |
|---|---|---|---|
| Entry | R substituent | (kcal mol⁻¹)^{a} | (min)^{b} |
| 1 | Perfluoro | −2.2 | 0.3 |
| 2 | 4-pyridine | −1.4 | 1 |
| 3 | p-SO₂CF₃ | −0.6 | 2 |
| 4 | p-C(O)CH₃ | −0.4 | 2 |
| 5 | Perchloro | −0.3 | 3 |
| 6 | p-C₆H₅ | −0.2 | 3 |
| 7 | p-Br | −0.1 | 3 |
| 8 | p-OCH₃ | 0.0 | 3 |
| 9 | H | 0.0 | 3 |
| 10 | m,m-NO₂ | 0.4 | 5 |
| 11 | 2-pyridine | 0.5 | 6 |
| 12 | p-NO₂ | 0.7 | 7 |
| 13 | benzothiazole | 1.1 | 11 |
| 14 | p-CF₃ | 1.2 | 12 |
| 15 | 3-pyridine | 1.2 | 12 |
| 16 | p-CN | 1.3 | 13 |
| 17 | p-CH₃ | 1.4 | 15 |
| 18 | p-F | 1.4 | 16 |
| 19 | p-Cl | 1.5 | 17 |
| 20 | p-CO₂CH₃ | 1.8 | 24 |
| 21 | p-N(CH₃)₂ | 2.2 | 36 |
| 22 | o,p-NO₂ | 3.5 | 32 |

^{a}calculations were done at the M08HX/6-311 + G**//B3LYP-D3BJ/6-31 + G* level in toluene (SMD method, ε = 2.3741) at 200° C. with a correction for concentration and quasi-harmonic factors.
ΔΔG^{‡} values are relative to the unsubstituted model compound (C).
^{b}times estimated to reach 99% conversion are first calculated from the Eyring equation to get the kinetic constant at the targeted temperature and then from the first-order integrated rate law.

### Example 18: Comparative Example of the Sulfinylation Performed in Batch Synthesis and Using a Continuous Flow Process According to the Invention

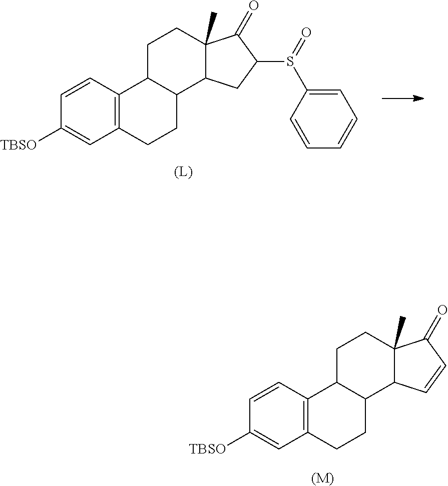

To a solution of 3-tert-butyldimethylsilyloxy-16-(phenylsulfinyl)-estra-1,3,5(10)-triene-17-one (L, 1.12 g, 2.20 mmol) in o-xylene (10.0 mL, 0.22 M) was added DMAD (0.8 mL, 6.60 mmol). The reaction mixture was stirred at 135° C. for 17 h and quenched by addition of water (15 mL). The phases were separated and the organic layer was dried over Na₂SO₄. After drying, 1 mL of Et₃N was added and the volatiles were removed under reduced pressure. A yield of 28% of compound (M) was detected by HPLC in the crude.

Following the procedure described in Example 1, a feed solution containing 0.22 M of compound (L) in toluene, in the presence of 1.5 equiv. of DMAD and 0.1 equiv. of triethylamine was subjected to thermolysis at 210° C. for 6 min residence time. 91% of compound (M) was detected by HPLC in the crude.

### Example 19: Pilot Scale Set Up

A solution of 3-tert-butyldimethylsilyloxy-16-(phenylsulfinyl)-estra-1,3,5(10)-triene-17-one (975 g, 1.96 mol) and Et₃N (26.1 mL, 0.20 mol) in toluene (8.0 L) was pumped with “pump A” (see Scheme 6) with a flow rate of 41.68 mL/min. A solution of DMAD (288 mL, 2.46 mol) in toluene (298 mL) was pumped with “pump B” with a flow rate of 3.05 mL/min. Flow A and Flow B were mixed and the combined flows were injected in a SS flow reactor (Vᵢₙₜ=134 mL) heated at 210° C. with a global flow rate of 44.73 mL/min for a residence time of 3 minutes. The feed was maintained liquid with a back-pressure regulator of 15 bar (220 psi). The flow was then cooled to 15° C. and the crude reaction mixture was retrieved in a flask. The organic layer was washed with a 1M aqueous solution of NaOH (8.0 L). The volatiles were removed under reduced pressure to give a brown crude solution containing an 88/5/3/3% area by HPLC of 3-tert-butyldimethylsilyloxy-estra-1,3,5(10)-15-tetraene-17-one/3-tert-butyldimethylsilyloxy-estra-1,3,5(10)-triene-17-one/3-tert-butyldimethylsilyloxy-estra-1,3,5(10)-14-tetraene-17-one/3-tert-butyldimethylsilyloxy-16-phenylthio-estra-1,3,5(10)-15-tetraene-17-one. Purification of the crude product by recrystallization in MTBE afforded 3-tert-butyldimethylsilyloxy-estra-1,3,5(10)-15-tetraene-17-one as an off-white solid (78%, 96.9% purity by HPLC).

### Example 20: Preparation of estra-1,3,5(10)-15-tetraene-17-one

### Step 1: 16-(phenylsulfinyl)-estra-1,3,5(10)-triene-17-one

16-(Phenylsulfinyl)-estra-1,3,5(10)-triene-17-one was prepared as described in step 2 of Example 7.

### Step 2: estra-1,3,5(10)-15-tetraene-17-one

To a solution of 16-(phenylsulfinyl)-estra-1,3,5(10)-triene-17-one (500 mg, 1.27 mmol) in N-methyl-2-pyrrolidone (12.6 mL) was added (2,2,6,6-tetramethylpiperidin-1-yl)oxyl (396 mg, 2.53 mmol). The solution was injected in a tubing (Vᵢₙₜ=5 mL) heated at 210° C. with a flow rate of 0.833 mL/min. The feed was maintained liquid with a back-pressure regulator (BPR) of 34 bar (500 psi). The flow was then cooled to 0° C. and the crude reaction mixture is retrieved in a flask. The product was obtained with a yield of 69% (determination by HPLC).

¹H-NMR (CDCl₃): δ 1.11 (3H, s, CH₃ at C-18), 1.57-1.87 (4H, m), 1.98-2.07 (1H, m), 2.13-2.25 (1H, m), 2.39-2.47 (1H, m), 2.51 (1H, d), 2.87-3.02 (2H, m), 4.66 (1H, s), 6.09 (1H, dd), 6.60 (1H, d), 6.65 (1H, dd), 7.15 (1H, d), 7.63 (1H, d).

1-15. (canceled)

16. A process for the preparation of a compound of formula (I) 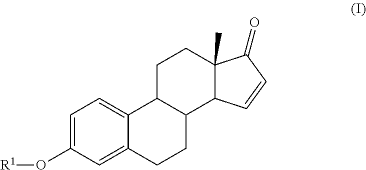
or a stereoisomer, a salt, a hydrate or a solvate thereof, comprising desulfinylation of a compound of formula (II) 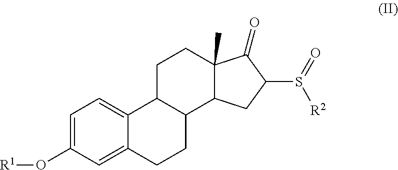
to produce a compound of formula (I), wherein:
R¹ is selected from hydrogen, R³Si(R⁴)(R⁵)—, C₁₋₆ alkyl, C₆₋₁₀ aryl, C₆₋₁₀ arylC₁₋₆ alkyl, —CH₂—CH═CR^{a}R^{b}, C₁₋₆ alkoxy, C₃₋₆ cycloalkyl, and R⁶CO—, wherein each of said C₁₋₆ alkyl, C₆₋₁₀ aryl, C₆₋₁₀ arylC₁₋₆ alkyl, and C₃₋₆ cycloalkyl may be optionally substituted with one or more substituents each independently selected from halo, C₁₋₆alkyl, haloC₁₋₆ alkyl, C₁₋₆ alkoxy, haloC₁₋₆ alkoxy, C₆₋₁₀ aryl, heterocyclyl, and nitro;
R² is C₆₋₁₀ aryl, or heteroaryl, wherein each C₆₋₁₀ aryl or heteroaryl may be optionally substituted with one or more substituents each independently selected from halo, C₁₋₆alkyl, haloC₁₋₆ alkyl, C₁₋₆ alkoxy, haloC₁₋₆ alkoxy, C₂₋₆ alkenyl, C₂₋₆ alkynyl, C₆₋₁₀ aryl, heterocyclyl, heteroaryl, hydroxyl, —S(O)₂R⁷, —S(O)R⁸, —CO₂R⁹, —C(O)R¹⁰, —SR¹³, —C(O)SR¹⁴, NR¹¹R¹², cyano and nitro;
R³, R⁴ and R⁵ are each independently C₁₋₆ alkyl or C₆₋₁₀ aryl, wherein each C₁₋₆ alkyl or C₆₋₁₀ aryl may be optionally substituted with one or more substituents each independently selected from halo, C₁₋₆ alkyl, haloC₁₋₆ alkyl, C₁₋₆ alkoxy, and haloC₁₋₆ alkoxy;
R⁶ is C₁₋₆ alkyl, C₆₋₁₀ aryl, or C₃₋₆ cycloalkyl, wherein each C₁₋₆ alkyl, C₆₋₁₀ aryl, or C₃₋₆ cycloalkyl may be optionally substituted by one or more substituents independently selected from halo, C₁₋₆ alkyl, haloC₁₋₆ alkyl, C₁₋₆ alkoxy, and haloC₁₋₆ alkoxy;
R⁷, R⁸, R⁹ and R¹⁰ are each independently selected from hydrogen, hydroxyl, C₁₋₆ alkyl, C₆₋₁₀aryl, and C₃₋₆cycloalkyl, wherein each C₁₋₆ alkyl, C₆₋₁₀ aryl, or C₃₋₆ cycloalkyl may be optionally substituted by one or more substituents independently selected from halo, C₁₋₆ alkyl, haloC₁₋₆ alkyl, C₁₋₆ alkoxy, and haloC₁₋₆ alkoxy;
R¹¹ and R¹² are each independently selected from the group consisting of hydrogen, C₁₋₆ alkyl, C₆₋₁₂ aryl, and C₃₋₆ cycloalkyl, wherein each C₁₋₆ alkyl, C₆₋₁₀ aryl, or C₃₋₆ cycloalkyl may be optionally substituted by one or more substituents independently selected from halo, C₁₋₆ alkyl, haloC₁₋₆ alkyl, C₁₋₆ alkoxy, and haloC₁₋₆ alkoxy;
R¹³ and R¹⁴ are each independently selected from hydrogen, C₁₋₆alkyl, C₆₋₁₀aryl, and C₃₋₆cycloalkyl, wherein each C₁₋₆ alkyl, C₆₋₁₀ aryl, or C₃₋₆ cycloalkyl may be optionally substituted by one or more substituents independently selected from halo, C₁₋₆ alkyl, haloC₁₋₆ alkyl, C₁₋₆ alkoxy, and haloC₁₋₆ alkoxy; and
R^{a} and R^{b} are each independently hydrogen, C₁₋₆ alkyl or C₆₋₁₀ aryl;
wherein said desulfinylation step is performed by a continuous flow process; and
wherein said desulfinylation step is performed by thermolysis at a thermolysis temperature (T) of at least 140° C.

17. The process according to claim 16, wherein said thermolysis is performed at a pressure (P) of at least 1 bar.

18. The process according to claim 16, wherein said desulfinylation step is performed for a thermolysis time (t) of a most 60 minutes.

19. The process according to claim 16, wherein said desulfinylation step is performed in at least one diluent.

20. The process according to claim 16, comprising:
(a) forming a mixture by contacting a compound of formula (II) with at least one diluent, optionally at least one scavenger, and optionally at least one base;
(b) feeding the mixture to an inlet of a channel of a reaction zone,
(c) driving a flow of the mixture along the channel from the inlet to an outlet, to form reaction products including at least one compound of formula (I);
(d) recovering the reaction products at the outlet, and optionally separating the at least one compound of formula (I) from other reaction products or keeping the compound(s) of formula (I) in the reaction products.

21. The process according to claim 20, wherein the reaction zone is a thermolysis reaction zone and step (c) comprises exposing the mixture to thermolysis by driving a flow of the mixture along the channel from the inlet to the outlet for a thermolysis time (t) at a pressure (P) and at a thermolysis temperature (T), to form thermolysis reaction products including at least one compound of formula (I).

22. The process according to claim 20, wherein compound of formula (II) is present in the mixture of step (a) in an amount of at least 0.01 mole/L of diluent.

23. The process according to claim 16, wherein the desulfinylation step is carried out in the presence of at least one scavenger.

24. The process according to claim 16, wherein the desulfinylation step is carried out in the presence of at least one scavenger, and wherein the at least one scavenger is present in an amount of at least 0.1 to at most 10.0 equivalents.

25. The process according to claim 16, wherein the desulfinylation step is carried out in the presence of at least one base.

26. The process according to claim 16, wherein compound of formula (II) is obtained by a process comprising:
(a) protecting the hydroxyl of a compound of formula (III) 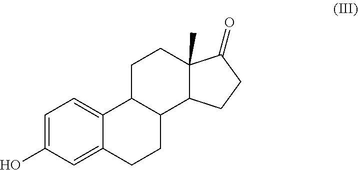 [SMILES: C[C@]12CCC3c4ccc(O)cc4CCC3C1CCC2=O]
to produce a compound of formula (IV) 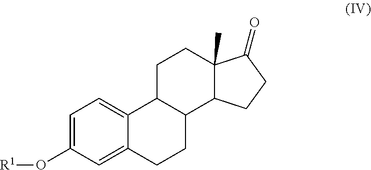
and
(b) sulfinylation of the compound of formula (IV) to produce the compound of formula (II).

27. A process for preparing estra-1,3,5(10),-triene 3,15α,16α,17β-tetrol of formula (E) 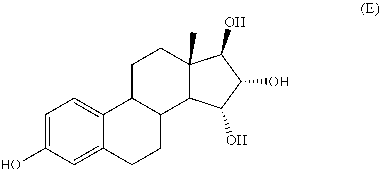 [SMILES: C[C@]12CCC3c4ccc(O)cc4CCC3C1[C@@H](O)[C@@H](O)[C@@H]2O]
or a stereoisomer, a salt, a hydrate, an ester, an ether, or a solvate thereof, comprising:
(I) preparing a compound of formula (I) 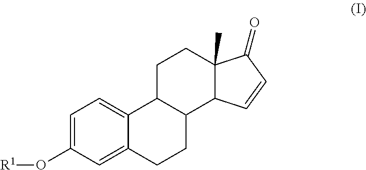
by a process comprising desulfinylation of a compound of formula (II) 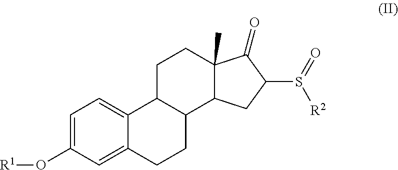
to produce a compound of formula (I), wherein:
R¹ is selected from hydrogen, R³Si(R⁴)(R⁵)—, C₁₋₆ alkyl, C₆₋₁₀ aryl, C₆₋₁₀ arylC₁₋₆ alkyl, —CH₂—CH═CR^{a}R^{b}, C₁₋₆ alkoxy, C₃₋₆ cycloalkyl, and R⁶CO—, wherein each of said C₁₋₆ alkyl, C₆₋₁₀ aryl, C₆₋₁₀ arylC₁₋₆ alkyl, and C₃₋₆ cycloalkyl may be optionally substituted with one or more substituents each independently selected from halo, C₁₋₆alkyl, haloC₁₋₆ alkyl, C₁₋₆ alkoxy, haloC₁₋₆ alkoxy, C₆₋₁₀ aryl, heterocyclyl, and nitro;
R² is C₆₋₁₀ aryl, or heteroaryl, wherein each C₆₋₁₀ aryl or heteroaryl may be optionally substituted with one or more substituents each independently selected from halo, C₁₋₆alkyl, haloC₁₋₆ alkyl, C₁₋₆ alkoxy, haloC₁₋₆ alkoxy, C₂₋₆ alkenyl, C₂₋₆ alkynyl, C₆₋₁₀ aryl, heterocyclyl, heteroaryl, hydroxyl, —S(O)₂R⁷, —S(O)R⁸, —CO₂R⁹, —C(O)R¹⁰, —SR¹³, —C(O)SR¹⁴, NR¹¹R¹², cyano and nitro;
R³, R⁴ and R⁵ are each independently C₁₋₆ alkyl or C₆₋₁₀ aryl, wherein each C₁₋₆ alkyl or C₆₋₁₀ aryl may be optionally substituted with one or more substituents each independently selected from halo, C₁₋₆ alkyl, haloC₁₋₆ alkyl, C₁₋₆ alkoxy, and haloC₁₋₆ alkoxy;
R⁶ is C₁₋₆ alkyl, C₆₋₁₀ aryl, or C₃₋₆ cycloalkyl, wherein each C₁₋₆ alkyl, C₆₋₁₀ aryl, or C₃₋₆ cycloalkyl may be optionally substituted by one or more substituents independently selected from halo, C₁₋₆ alkyl, haloC₁₋₆ alkyl, C₁₋₆ alkoxy, and haloC₁₋₆ alkoxy;
R⁷, R⁸, R⁹ and R¹⁰ are each independently selected from hydrogen, hydroxyl, C₁₋₆ alkyl, C₆₋₁₀aryl, and C₃₋₆cycloalkyl, wherein each C₁₋₆ alkyl, C₆₋₁₀ aryl, or C₃₋₆ cycloalkyl may be optionally substituted by one or more substituents independently selected from halo, C₁₋₆ alkyl, haloC₁₋₆ alkyl, C₁₋₆ alkoxy, and haloC₁₋₆ alkoxy;
R¹¹ and R¹² are each independently selected from the group consisting of hydrogen, C₁₋₆ alkyl, C₆₋₁₂ aryl, and C₃₋₆ cycloalkyl, wherein each C₁₋₆ alkyl, C₆₋₁₀ aryl, or C₃₋₆ cycloalkyl may be optionally substituted by one or more substituents independently selected from halo, C₁₋₆ alkyl, haloC₁₋₆ alkyl, C₁₋₆ alkoxy, and haloC₁₋₆ alkoxy;
R¹³ and R¹⁴ are each independently selected from hydrogen, C₁₋₆alkyl, C₆₋₁₀aryl, and C₃₋₆cycloalkyl, wherein each C₁₋₆ alkyl, C₆₋₁₀ aryl, or C₃₋₆ cycloalkyl may be optionally substituted by one or more substituents independently selected from halo, C₁₋₆ alkyl, haloC₁₋₆ alkyl, C₁₋₆ alkoxy, and haloC₁₋₆ alkoxy; and
R^{a} and R^{b} are each independently hydrogen, C₁₋₆ alkyl or C₆₋₁₀ aryl;
wherein said desulfinylation step is performed by a continuous flow process; and
wherein said desulfinylation step is performed by thermolysis at a thermolysis temperature (T) of at least 140° C., and
(II) reacting the compound of formula (I) to produce estra-1,3,5(10),-triene 3,15α,16α,17β-tetrol of formula (E), or a stereoisomer, a salt, a hydrate, an ester, an ether, or a solvate thereof.

28. A composition comprising a compound of formula (I) and a compound of formula (IIa) 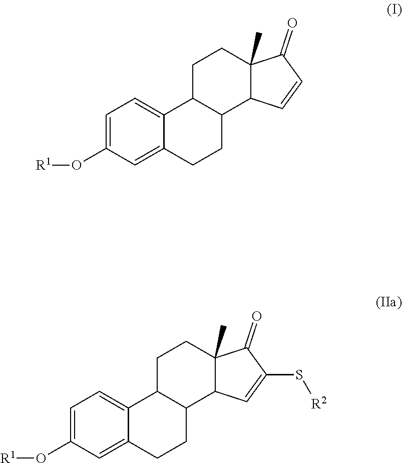
wherein:
R¹ is selected from hydrogen, R³Si(R⁴)(R⁵)—, C₁₋₆ alkyl, C₆₋₁₀ aryl, C₆₋₁₀ arylC₁₋₆ alkyl, —CH₂—CH═CR^{a}R^{b}, C₁₋₆ alkoxy, C₃₋₆ cycloalkyl, and R⁶CO—, wherein each of said C₁₋₆ alkyl, C₆₋₁₀ aryl, C₆₋₁₀ arylC₁₋₆ alkyl, and C₃₋₆ cycloalkyl may be optionally substituted with one or more substituents each independently selected from halo, C₁₋₆alkyl, haloC₁₋₆ alkyl, C₁₋₆ alkoxy, haloC₁₋₆ alkoxy, C₆₋₁₀ aryl, heterocyclyl, and nitro;
R² is C₆₋₁₀ aryl, or heteroaryl, wherein each C₆₋₁₀ aryl or heteroaryl may be optionally substituted with one or more substituents each independently selected from halo, C₁₋₆alkyl, haloC₁₋₆ alkyl, C₁₋₆ alkoxy, haloC₁₋₆ alkoxy, C₂₋₆ alkenyl, C₂₋₆ alkynyl, C₆₋₁₀ aryl, heterocyclyl, heteroaryl, hydroxyl, —S(O)₂R⁷, —S(O)R⁸, —CO₂R⁹, —C(O)R¹⁰, —SR¹³, —C(O)SR¹⁴, NR¹¹R¹² cyano and nitro;
R³, R⁴ and R⁵ are each independently C₁₋₆ alkyl or C₆₋₁₀ aryl, wherein each C₁₋₆ alkyl or C₆₋₁₀ aryl may be optionally substituted with one or more substituents each independently selected from halo, C₁₋₆ alkyl, haloC₁₋₆ alkyl, C₁₋₆ alkoxy, and haloC₁₋₆ alkoxy;
R⁶ is C₁₋₆ alkyl, C₆₋₁₀ aryl, or C₃₋₆ cycloalkyl, wherein each C₁₋₆ alkyl, C₆₋₁₀ aryl, or C₃₋₆ cycloalkyl may be optionally substituted by one or more substituents independently selected from halo, C₁₋₆ alkyl, haloC₁₋₆ alkyl, C₁₋₆ alkoxy, and haloC₁₋₆ alkoxy;
R⁷, R⁸, R⁹ and R¹⁰ are each independently selected from hydrogen, hydroxyl, C₁₋₆ alkyl, C₆₋₁₀aryl, and C₃₋₆cycloalkyl, wherein each C₁₋₆ alkyl, C₆₋₁₀ aryl, or C₃₋₆ cycloalkyl may be optionally substituted by one or more substituents independently selected from halo, C₁₋₆ alkyl, haloC₁₋₆ alkyl, C₁₋₆ alkoxy, and haloC₁₋₆ alkoxy;
R¹¹ and R¹² are each independently selected from the group consisting of hydrogen, C₁₋₆ alkyl, C₆₋₁₂ aryl, and C₃₋₆ cycloalkyl, wherein each C₁₋₆ alkyl, C₆₋₁₀ aryl, or C₃₋₆ cycloalkyl may be optionally substituted by one or more substituents independently selected from halo, C₁₋₆ alkyl, haloC₁₋₆ alkyl, C₁₋₆ alkoxy, and haloC₁₋₆ alkoxy;
R¹³ and R¹⁴ are each independently selected from hydrogen, C₁₋₆alkyl, C₆₋₁₀aryl, and C₃₋₆cycloalkyl, wherein each C₁₋₆ alkyl, C₆₋₁₀ aryl, or C₃₋₆ cycloalkyl may be optionally substituted by one or more substituents independently selected from halo, C₁₋₆ alkyl, haloC₁₋₆ alkyl, C₁₋₆ alkoxy, and haloC₁₋₆ alkoxy; and
R^{a} and R^{b} are each independently hydrogen, C₁₋₆ alkyl or C₆₋₁₀ aryl.
